(19) United States
(12) Patent Application Publication
(10) Pub. No.: US 2016/0319362 A1
(43) Pub. Date: Nov. 3, 2016

(54) METHOD FOR DIAGNOSING HEMATOLOGICAL MALIGNANCIES AND ASSOCIATED KIT

(71) Applicants: UNIVERSITE DE ROUEN, Mont-Saint-Aignan (FR); CENTRE HENRI BECQUEREL, Rouen (FR); INSTITUT NATIONAL DE LA SANTÉ ET LA RECHERCHE MÉDICALE, Paris Cedex 13 (FR)

(72) Inventors: Philippe RUMINY, Rouen (FR); Vinciane MARCHAND, Rouen (FR); Fabrice JARDIN, Rouen (FR)

(21) Appl. No.: 14/917,087

(22) PCT Filed: Sep. 11, 2014
(86) PCT No.: PCT/FR2014/052255

§ 371 Date: Mar. 7, 2016

(30) Foreign Application Priority Data
Sep. 11, 2013 (FR) 13 58721

(51) Int. Cl. C12Q 1/68

(52) U.S. Cl. CPC C12Q 1/6886

(57) ABSTRACT

A method for diagnosing a cancer in a subject, notably with the aim of finding fusion transcripts, includes an RT-MLPA step carried out on a biological sample obtained from the subject using the probes SEQ ID NO: 1 to 25, 30, 31 and 113 to 120, and/or with at least the probes SEQ ID NO: 374 to 405, and/or with at least the probes SEQ ID NO: 524 to 559, each of the probes being fused, at one end at least, with a priming sequence.

The present invention relates to a method for diagnosing a cancer in a subject, comprising a step of RT-MLPA on a biological sample obtained from said subject using at least one pair of probes selected from:

probes SEQ ID NO: 1 to 25, 30, 31 and 113 to 120,

probes SEQ ID NO: 374 to 405, and

probes SEQ ID NO: 524 to 559,

each of the probes being fused, at one end at least, with a priming sequence.

Preferably, the present invention relates to a method for diagnosing a cancer in a subject, comprising an RT-MLPA step carried out on a biological sample obtained from said subject, notably with the aim of finding fusion transcripts, using the probes SEQ ID NO: 1 to 25, 30, 31 and 113 to 120, and/or using the probes SEQ ID NO: 374 to 405, and/or using at least the probes SEQ ID NO: 524 to 559, each of the probes being fused, at one end at least, with a priming sequence. The present invention also relates to a kit comprising at least the probes SEQ ID NO: 1 to 25, 30, 31 and 113 to 120, and/or at least the probes SEQ ID NO: 374 to 405, and/or at least the probes SEQ ID NO: 524 to 559, each of the probes being fused, at one end at least, with a priming sequence.

Cancers are due to accumulation of genetic abnormalities by tumor cells. These abnormalities include many chromosome rearrangements (translocations, deletions and inversions) which lead to the formation of fusion genes. These fusion genes are often associated with particular forms of tumor, and detection of them may contribute significantly to the making of a diagnosis (The impact of translocations and gene fusions on cancer causation. Mitelman F, Johansson B, Mertens F., Nat Rev Cancer. 2007 April; 7(4): 233-45). They are also often used as molecular markers for monitoring the efficacy of treatments and tracing the evolution of the disease, for example in acute leukemias (Standardized RT-PCR analysis of fusion gene transcripts from chromosome aberrations in acute leukemia for detection of minimal residual disease. Report of the BIOMED-1 Concerted Action: investigation of minimal residual disease in acute leukemia. van Dongen J J, Macintyre E A, Gabert J A, Delabesse E, Rossi V, Saglio G, Gottardi E, Rambaldi A, Dotti G, Griesinger F, Parreira A, Gameiro P, Diáz M G, Malec M, Langerak A W, San Miguel J F, Biondi A. Leukemia. 1999 December; 13(12): 1901-28).

Today, the two main techniques for finding these fusion genes are cytogenetics and RT-PCR.

Cytogenetics consists of establishing the karyotype of the cancer cells for finding any abnormalities of number and/or of structure of the chromosomes. It has the advantage of providing an overall view of the whole genome. It is, however, of relatively low sensitivity, and its efficacy is strongly dependent on the percentage of tumor cells in the sample to be analyzed and the possibility of obtaining viable cell cultures. Another of its drawbacks is its low resolution, which does not allow certain rearrangements to be detected (in particular small inversions and deletions). Finally, certain tumors are associated with a major genomic instability that masks the pathognomonic genetic abnormalities. Analysis of the karyotypes is therefore often difficult and can only be carried out by personnel possessing excellent expertise.

The second technique, RT-PCR, is carried out starting from RNA extracted from the tumor cells. It has excellent sensitivity, far higher than cytogenetics. This sensitivity makes it the reference technique for analyzing biological samples where the percentage of tumor cells is low, which makes it possible to monitor the efficacy of the treatments or anticipate any relapses very early. However, its main limitation is connected with the fact that it is extremely difficult to multiplex analyses of this type. As each translocation generally has to be found by a specific test, only a few recurrent fusions among the numerous that are now known are investigated in routine analysis laboratories.

Therefore there is now a need for a specific, sensitive test, which is also simple and quick to implement, allowing accurate diagnosis of a cancer, notably a solid tumor (sarcoma or carcinoma) or a leukemia.

Surprisingly, the inventors have succeeded in developing an assay for diagnosing a specific cancer, notably a solid tumor (for example sarcoma or carcinoma) or a leukemia. This assay allows simultaneous investigation for a very large number of chromosome rearrangements in many forms of cancer, both economically and quickly (the assay may be carried out in one day). Moreover, its simplicity of use allows a person who has mastered the conventional techniques of molecular biology to perform all of the steps without special training, using equipment already available in most routine diagnostic laboratories.

The invention therefore relates to a molecular assay notably comprising an RT-MLPA step (Reverse Transcriptase Ligation-Dependent Probe Amplification), performed in multiplex mode. The multiplex mode gives a saving of time, as it is quicker than several monoplex modes, and is economically advantageous. It also allows simultaneous investigation for a much higher number of abnormalities than the techniques currently available.

The present invention therefore relates to a method for diagnosing a cancer in a subject, comprising a step of RT-MLPA on a biological sample obtained from said subject, using at least one pair of probes selected from:

probes SEQ ID NO: 1 to 25, 30, 31 and 113 to 120,

probes SEQ ID NO: 374 to 405, and

probes SEQ ID NO: 524 to 559,

each of the probes being fused, at one end at least, with a priming sequence. Preferably, said pair of probes consists of two probes that hybridize side by side during the RT-MLPA step.

Preferably, the present invention relates to a method for diagnosing a cancer in a subject, comprising an RT-MLPA step carried out on a biological sample obtained from said subject using the probes SEQ ID NO: 1 to 25, 30, 31 and 113 to 120, and/or using the probes SEQ ID NO: 374 to 405, and/or at least the probes SEQ ID NO: 524 to 559, each of the probes being fused, at one end at least, with a priming sequence.

The present invention also relates to a kit comprising at least the probes SEQ ID NO: 1 to 25, 30, 31 and 113 to 120, and/or the probes SEQ ID NO: 374 to 405, and/or at least the probes SEQ ID NO: 524 to 559, preferably further comprising the probes SEQ ID NO: 26 to 29, 66 to 112 and 121 to 219, and/or the probes SEQ ID NO: 438 to 480, and/or the probes SEQ ID NO: 616 to 674, and/or the probes SEQ ID NO: 734 to 741, and/or the probes SEQ ID NO: 750 to 774, each of the probes being fused, at one end at least, with a priming sequence. Preferably, the kit comprises at least the probes SEQ ID NO: 35 to 59, 64, 65 and 267 to 274, and/or the probes SEQ ID NO: 406 to 437, and/or the probes SEQ ID NO: 560 to 595, preferably it further comprises the probes SEQ ID NO: 60 to 63, 220 to 266 and 275 to 373, and/or the probes SEQ ID NO: 675 to 733, and/or the probes SEQ ID NO: 775 to 799, and/or the probes SEQ ID NO: 742 to 749.

“MLPA” means Multiplex Ligation-Dependent Probe Amplification, which allows simultaneous amplification of several targets of interest contiguous with one another, using one or more specific probes. This technique is very advantageous, in the context of the present invention, for determining the presence of translocations, which are frequent in malignant tumors.

“RT-MLPA” means Multiplex Ligation-Dependent Probe Amplification preceded by Reverse Transcription (RT), which makes it possible, in the context of the present invention, to start from the RNA of a subject to amplify and characterize the fusion genes of interest.

“Subject” means an individual who is healthy or may have a cancer or for screening, diagnosis or monitoring.

“Biological sample” means a sample containing biological material. More preferably, it means any sample containing RNA. This sample may be obtained from biological sampling carried out on a living being (human patient, animal, plant). Preferably, the biological samples according to the invention are selected from whole blood, bone marrow and a biopsy, obtained from a subject, notably human.

“Sensitivity” means the proportion of positive tests in subjects with cancers and who have the abnormalities under investigation.

“Specificity” means the proportion of negative tests in subjects without cancers and who do not have the abnormalities under investigation.

“Cancer” means a disease characterized by an abnormally high level of cellular proliferation within a normal tissue of the organism, so that the latter's survival is threatened. According to the invention, the cancer is preferably selected from leukemias, sarcomas and carcinomas. Leukemias are cancers of the cells of the bone marrow, and of the lymphoid and myeloid systems. Sarcomas are malignant tumors that have developed at the expense of common extraskeletal connective tissue such as adipose tissue, muscle tissue, vessels and the peripheral nervous system. Carcinomas are malignant tumors that have developed at the expense of epithelial tissue.

“Probe” means a nucleic acid sequence with a length of between 15 and 40 nucleotides, preferably between 20 and 25 nucleotides, and complementary to a cDNA sequence from an RNA of the subject (endogenous). It is therefore capable of hybridizing to said cDNA sequence from an RNA of the subject.

“Priming sequence” means a nucleic acid sequence with a length of between 15 and 30 nucleotides, preferably between 20 and 25 nucleotides, and not complementary to the cDNA sequences from the subject's RNA. Therefore it is not complementary to the cDNA corresponding to the endogenous RNA. Therefore it cannot hybridize to said cDNA sequences. Preferably, the priming sequence is selected from the sequences SEQ ID NO: 33 and SEQ ID NO: 34.

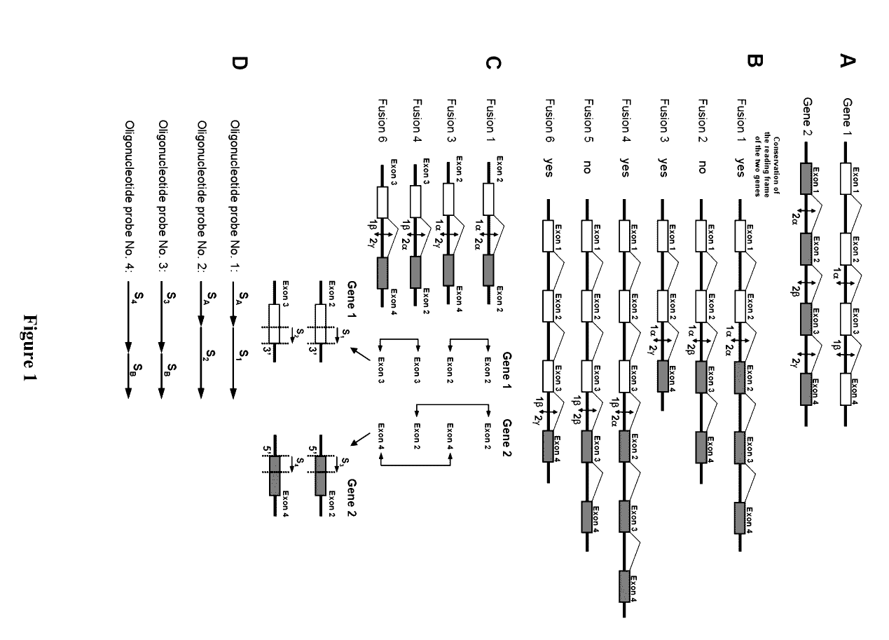

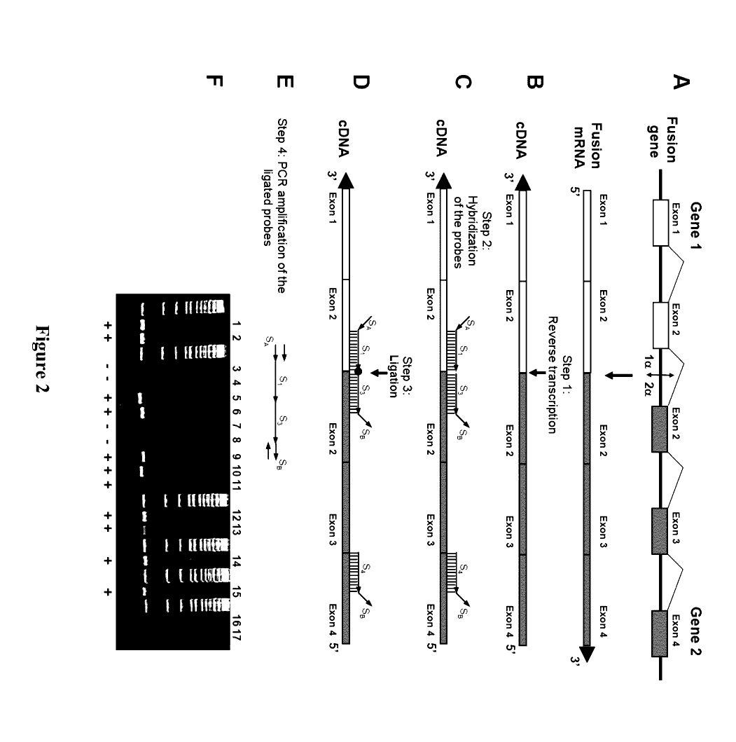

The inventors have identified specific probes for each type of translocation observed in certain cancers. This identification is based on analysis of the intron-exon structure of the genes involved in the translocations, as is shown in FIG. 1. In particular, the points of breaks that may lead to expression of functional chimeric proteins are sought (FIG. 1B). Based on these results, DNA sequences with 25 to 40 base pairs are defined, precisely corresponding to the 5′ and 3′ ends of the exons of the two genes juxtaposed after splicing of the hybrid transcripts (FIG. 1C). A set of probes is then defined as follows: a priming sequence (S_{A} in FIG. 1) with about twenty base pairs is added at 5′ of all the probes complementary to the exons of the genes forming the 5′ portion of the fusion transcripts. A second priming sequence (S_{B} in FIG. 1), also with about twenty base pairs but different from S_{A}, is added to the 3′ ends of all the probes complementary to the exons of the genes forming the 3′ portion of the fusion transcripts (FIG. 1D). These probes are then combined in a mixture, and contain all the elements required for detecting one or more fusion transcripts, produced by one or more translocations.

The probes used in the invention are therefore capable of hybridizing either to the last nucleotides of the last exon at 5′ of the translocation, or to the first nucleotides of the first exon at 3′ of the translocation. Preferably, the probes used in the invention that are capable of hybridizing to the first nucleotides of the first exon at 3′ of the translocation are phosphorylated at 5′ before they are used.

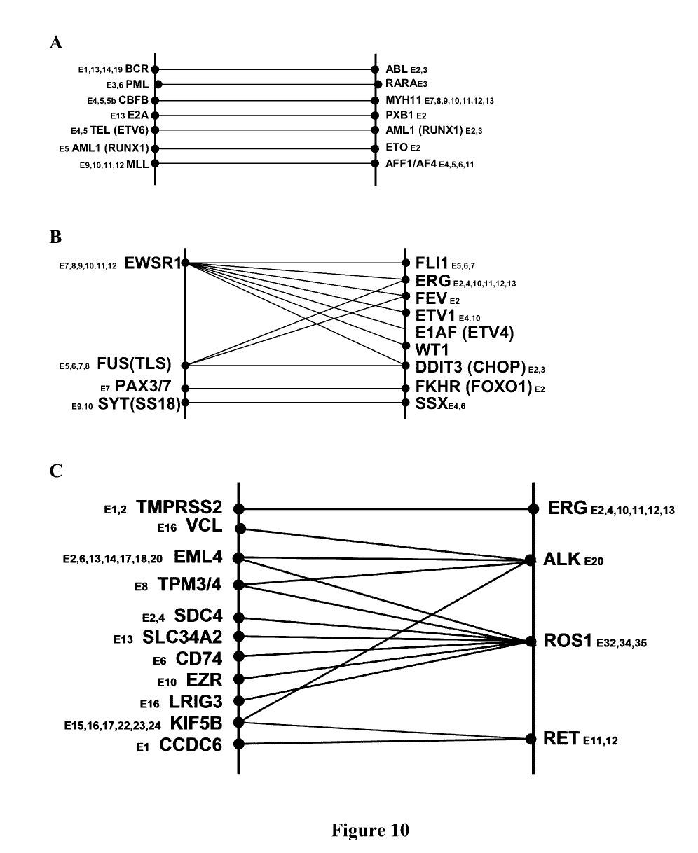

The various translocations identified according to the present invention are illustrated in FIGS. 10 to 12.

The probes according to the invention may be the sequences SEQ ID NO: 1 to 25, 30, 31 and 113 to 120, optionally combined with the probes SEQ ID NO: 26 to 29, 66 to 112 and 121 to 219 and/or with the probes SEQ ID NO: 616 to 674.

The probes according to the invention may be the sequences SEQ ID NO: 374 to 405, optionally combined with the probes SEQ ID NO: 438 to 480 and/or at least with the probes SEQ ID NO: 750 to 774.

The probes according to the invention may be the sequences SEQ ID NO: 524 to 559, optionally combined with the probes SEQ ID NO: 438 to 480 and/or at least with the probes SEQ ID NO: 374 to 405 and/or with the probes SEQ ID NO: 734 to 741.

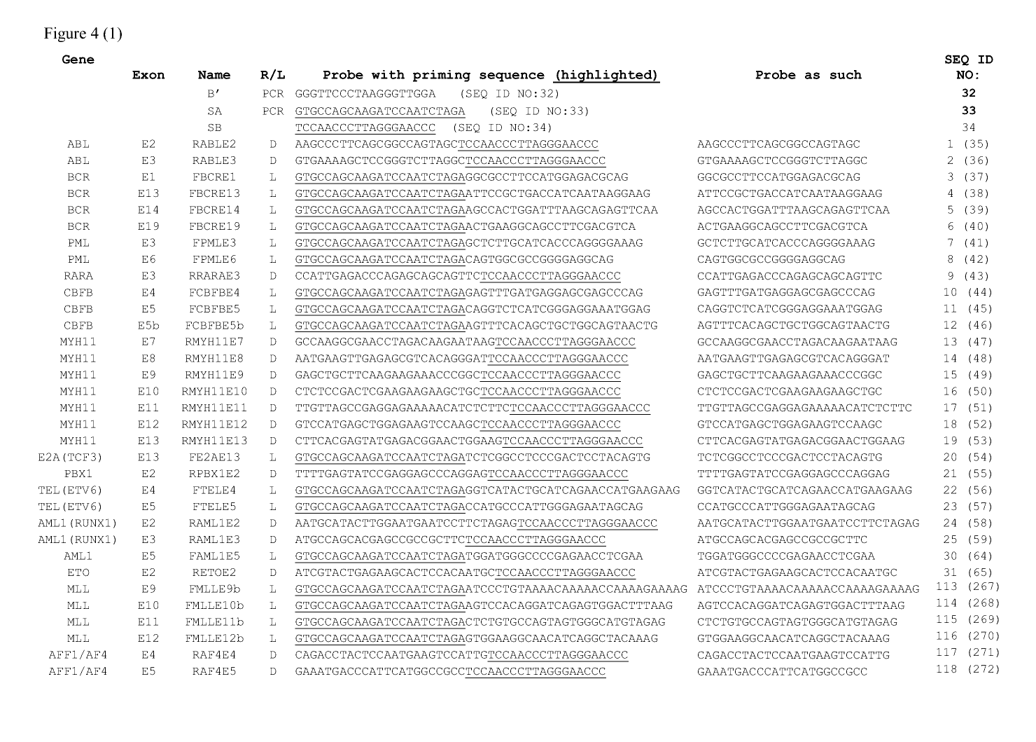

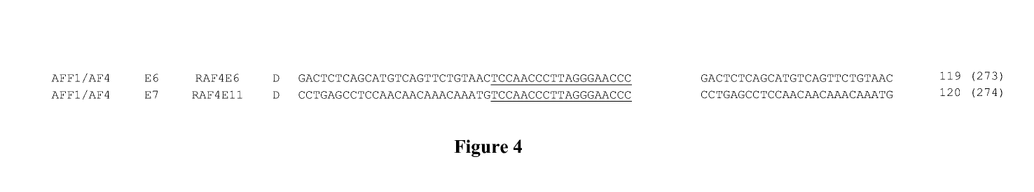

FIGS. 4, 5, and 7 to 9 give details of the name of each probe, their position (i.e. whether they are at 5′ (L) or at 3′ (R) of the abnormal junction), their structure (presence or absence of a priming sequence, and characteristics of this sequence), as well as the gene and the exon to which each probe hybridizes.

Preferably, each of the probes SEQ ID NO: 3 to SEQ ID NO: 8; SEQ ID NO: 10 to SEQ ID NO: 12; SEQ ID NO: 20, SEQ ID NO: 22, SEQ ID NO: 23, SEQ ID NO: 26 and SEQ ID NO: 30 is fused at 5′ with a priming sequence, and

each of the probes SEQ ID NO: 1, SEQ ID NO: 2, SEQ ID NO: 9, SEQ ID NO: 13 to SEQ ID NO: 19; SEQ ID NO: 21, SEQ ID NO: 24, SEQ ID NO: 25, SEQ ID NO: 27 to SEQ ID NO: 29; and SEQ ID NO: 31 is fused at 3′ with a different priming sequence.

Preferably, the probes fused with the priming sequences usable according to the invention are the sequences SEQ ID NO: 35 to 59, 64, 65 and 267 to 274, optionally combined with the probes SEQ ID NO: 60 to 63, 220 to 266 and 275 to 373 and/or with the probes SEQ ID NO: 675 to 733.

Preferably, the probes fused with the priming sequences usable according to the invention are the sequences SEQ ID NO: 406 to 437, optionally combined with the probes SEQ ID NO: 481 to 523 and/or with the probes SEQ ID NO: 775 to 799.

Preferably, the probes fused with the priming sequences usable according to the invention are the sequences SEQ ID NO: 560 to 595, optionally combined with the probes SEQ ID NO: 481 to 523 and/or with the probes SEQ ID NO: 742 to 749.

The method of diagnosis according to the invention is carried out with at least one pair of probes selected from:

probes SEQ ID NO: 1 to 25, 30, 31 and 113 to 120,

probes SEQ ID NO: 374 to 405, and

probes SEQ ID NO: 524 to 559,

each of the probes being fused, at one end at least, with a priming sequence.

The pair of probes may be selected from the probes with a specific sequence described above, and as is explained in FIGS. 4, 7 and 9. “Pair of probes” means a set of two probes, one being situated at 5′ (“L” in FIGS. 4-5, and 7-9) of the translocation or gene mutation, the other being situated at 3′ (“R” in FIGS. 4-5, and 7-9) of the translocation or gene mutation.

Preferably, the method of diagnosis according to the invention relates to the diagnosis of leukemias, and comprises an RT-MLPA step carried out on a biological sample obtained from said subject using the probes SEQ ID NO: 1 to 25, 30, 31 and 113 to 120, each of the probes being fused, at one end at least, with a priming sequence.

The leukemias are preferably selected from B-cell acute lymphoblastic leukemias, T-cell acute lymphoblastic leukemias, acute myeloblastic leukemias, chronic myeloid leukemias, lymphomas and myelomas.

Preferably, in this case, the method of diagnosis according to the invention also uses the probes SEQ ID NO: 26 to 29, 66 to 112 and 121 to 219, and/or the probes SEQ ID NO: 616 to 674, for the RT-MLPA step, each of the probes being fused, at one end at least, with a priming sequence. Thus, in this case, a mixture of probes SEQ ID NO: 1 to 25, 30, 31 and 113 to 120, and SEQ ID NO: 26 to 29, 66 to 112 and 121 to 219 and/or SEQ ID NO: 616 to 674 is used, each of the probes being fused, at one end at least, with a priming sequence.

Preferably, the invention relates to a diagnostic kit for leukemias comprising the probes SEQ ID NO: 1 to 25, 30, 31 and 113 to 120, and 26 to 29, 66 to 112 and 121 to 219, and/or SEQ ID NO: 616 to 674, each of the probes being fused, at one end at least, with a priming sequence. Preferably, said kit comprises the probes SEQ ID NO: 35 to 59, 64, 65 and 267 to 274, and preferably also probes SEQ ID NO: 60 to 63, 220 to 266 and 275 to 373, and/or preferably also probes SEQ ID NO: 675 to 733.

Preferably, the method of diagnosis according to the invention relates to the diagnosis of sarcomas, and comprises a step of RT-MLPA in a biological sample obtained from said subject using the probes SEQ ID NO: 374 to 405, each of the probes being fused, at one end at least, with a priming sequence.

The sarcomas are preferably selected from Ewing sarcomas, rhabdomyosarcomas, desmoplastic round-cell tumors, synovial sarcomas and myxoid liposarcomas.

Preferably, in this case, the method of diagnosis according to the invention also uses the probes SEQ ID NO: 438 to 480 and/or probes SEQ ID NO: 750 to 774, for the RT-MLPA step, each of the probes being fused, at one end at least, with a priming sequence. Thus, in this case, a mixture of probes SEQ ID NO: 374 to 405, and 438 to 480 and/or 750 to 774, is used, each of the probes being fused, at one end at least, with a priming sequence.

Preferably, the invention relates to a diagnostic kit for sarcomas comprising the probes SEQ ID NO: 374 to 405, each of the probes being fused, at one end at least, with a priming sequence. Preferably, said kit comprises the probes SEQ ID NO: 406 to 437, and preferably also probes SEQ ID NO: 481 to 523, and/or preferably also probes SEQ ID NO: 775 to 799.

Preferably, the method of diagnosis according to the invention relates to the diagnosis of carcinomas, and comprises a step of RT-MLPA in a biological sample obtained from said subject using the probes SEQ ID NO: 524 to 559, each of the probes being fused, at one end at least, with a priming sequence.

The carcinomas are preferably selected from nonsmall cell bronchopulmonary carcinomas, prostatic adenocarcinomas, carcinomas of the kidney, thyroid and breast.

Preferably, in this case, the method of diagnosis according to the invention also uses the probes SEQ ID NO: 438 to 480 and/or probes SEQ ID NO: 734 to 741, for the RT-MLPA step, each of the probes being fused, at one end at least, with a priming sequence. Thus, in this case, a mixture of probes SEQ ID NO: 524 to 559, and 438 to 480 and/or 734 to 741, is used, each of the probes being fused, at one end at least, with a priming sequence.

Preferably, the invention relates to a diagnostic kit for carcinomas comprising the probes SEQ ID NO: 524 to 559, each of the probes being fused, at one end at least, with a priming sequence. Preferably, said kit comprises the probes SEQ ID NO: 560 to 595, and preferably also probes SEQ ID NO: 481 to 523, and/or preferably also probes SEQ ID NO: 742 to 749.

Preferably the method of the invention uses a biological sample selected from whole blood, bone marrow and a biopsy obtained from the subject.

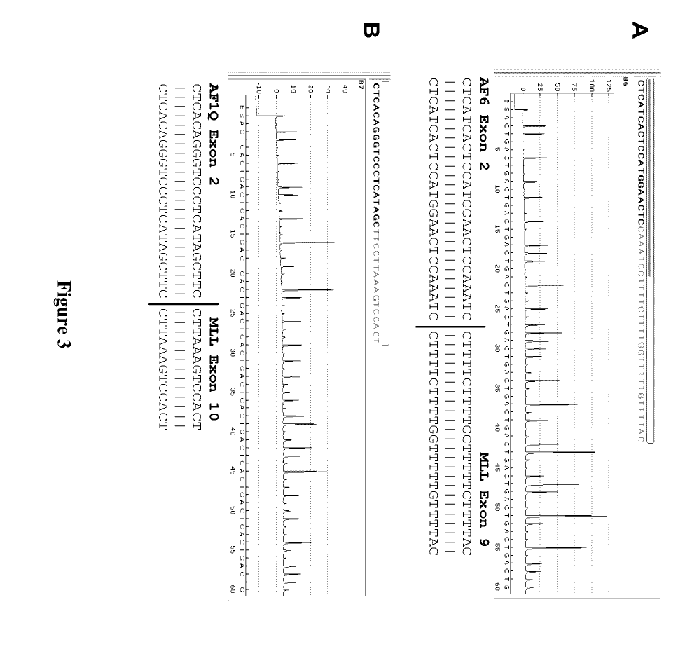

The RT-MLPA step is derived from MLPA (Multiplex Ligation-Dependent Probe Amplification), notably described in U.S. Pat. No. 6,955,901. It allows simultaneous detection and assay of a large number of different oligonucleotide sequences. The principle is as follows (see FIG. 2): RNA extracted from tumor tissue is first converted to complementary DNA (cDNA) by reverse transcription. This cDNA is then incubated with a mixture of suitable probes, and each can then hybridize to the sequences of the exons to which they correspond. If one of the fusion transcripts sought is present in the sample, two probes will bind side by side to the corresponding cDNA. A ligation reaction is then performed using an enzyme with DNA ligase activity, which establishes a covalent bond between the two contiguous probes. A PCR reaction (Polymerase Chain Reaction) is then carried out, using primers corresponding to the priming sequences (S_{A} and S_{B} in FIG. 2), which makes it possible to amplify the two ligated probes specifically.

Obtaining an amplification product after the RT-MLPA step indicates that one of the translocations sought is present in the sample analyzed.

Preferably, the RT-MLPA step used in the method according to the invention comprises at least the following steps:

a) extraction of RNA from the biological sample from the subject;
b) conversion of the RNA extracted in a) to cDNA by reverse transcription;
c) incubation of the cDNA obtained in b) with at least one pair of probes selected from:

probes SEQ ID NO: 1 to 25, 30, 31 and 113 to 120,

probes SEQ ID NO: 374 to 405, and

probes SEQ ID NO: 524 to 559,

each of the probes being fused, at one end at least, with a priming sequence. Preferably, in this step c), mixtures of probes as described above are used.
Preferably, in this step c), the cDNA obtained in b) is incubated with at least the probes SEQ ID NO: 1 to 25, 30, 31 and 113 to 120, and/or with at least the probes SEQ ID NO: 374 to 405, and/or with at least the probes SEQ ID NO: 524 to 559, each of the probes being fused, at one end at least, with a priming sequence;
d) addition of a DNA ligase to the mixture obtained in c), in order to establish a covalent bond between two contiguous probes;
e) PCR amplification of the covalently bound contiguous probes obtained in d).

Typically, extraction of RNA from the biological sample according to step a) is performed by the conventional techniques that are familiar to a person skilled in the art. For example, this extraction may be performed by cellular lysis of the cells obtained from the biological sample. This lysis may be of a chemical, physical or thermal nature. This cellular lysis is generally followed by a purification step for separating and concentrating the nucleic acids from other cellular debris. Commercial kits of the QIAGEN and Zymo Research type, or else those marketed by Invitrogen, may be used for carrying out step a). Of course, the various relevant techniques differ depending on the nature of the biological sample being assayed. The knowledge of a person skilled in the art allows him to easily adapt these steps of lysis and purification to said biological sample being tested.

Preferably, the RNA extracted in step a) is then converted by reverse transcription to cDNA; this is step b) (see FIG. 2B). This step b) may be carried out using any technique of reverse transcription known from the prior art. It may notably be done using reverse transcriptase marketed by Qiagen, Promega or Ambion, according to the classical conditions for use, or else using M-MLV Reverse Transcriptase from Invitrogen.

Preferably, the cDNA obtained in step b) is then incubated with at least the probes SEQ ID NO: 1 to 25, 30, 31 and 113 to 120, and/or with at least the probes SEQ ID NO:

374 to 405, and/or with at least the probes SEQ ID NO: 524 to 559, each of the probes being fused, at one end at least, with a priming sequence. This is step c) of hybridization of the probes (see FIG. 2C). In fact, the probes, which are complementary to a portion of cDNA, will hybridize to this portion if the latter is present in the cDNA. As is shown in FIG. 2C, owing to their sequence, the probes will therefore hybridize:
either to the portion of cDNA corresponding to the last nucleotides of the last exon at 5′ of the translocation. They are then “F” or “Forward” probes, also called “L” or “Left”;
or to the portion of cDNA corresponding to the first nucleotides of the first exon at 3′ of the translocation. They are then “R” or “Reverse” probes, also called “R” or “Right”.
   At the end of step c), the probes hybridized to cDNA are contiguous, if and only if the translocation has taken place.
   This step c) is typically carried out by incubating the cDNA and the mixture of probes at a temperature between 90° C. and 100° C., for a time of from 1 to 5 minutes, and then leaving them to incubate for at least 1 h, preferably 16 h, at a temperature of about 60° C. It may be carried out using the commercial kit sold by the company MRC-Holland (SALSA MLPA Buffer).

At the end of step c), a DNA ligase is typically added to bind covalently only the contiguous probes; this is step d) (see FIG. 2D). The DNA ligase is notably ligase 65, sold by MRC-Holland, Amsterdam, the Netherlands (SALSA Ligase-65). This step d) is typically carried out using the kits Lig-5a, Lig-10 or Lig-50 from MRC-Holland, Amsterdam, the Netherlands. It is typically carried out by incubating the DNA ligase and the mixture obtained in step c) at a temperature between 50° C. and 60° C., for a time of from 10 to 20 minutes, and then for a time of from 2 to 10 minutes at a temperature between 95° C. and 100° C.

At the end of step d), each pair of contiguous probes L and R is bound covalently, and the priming sequence of each probe is still present at 5′ and at 3′.

Preferably, the method also comprises a step e) of PCR amplification of the covalently bound contiguous probes obtained in d) (see FIG. 2E). This PCR step is carried out using a primer pair, one of the primers being identical to the priming sequence at 5′, and the other primer being complementary to the priming sequence at 3′. Preferably, the PCR amplification in step e) is carried out using the primers SEQ ID NO: 32 and 33, preferably one being labeled at its 5′ end with a biotin, in order to allow step (f). PCR is typically carried out using commercial kits, such as the ready-to-use kits sold by Eurogentec (Red‘y’Star Mix). Typically, PCR takes place in a first phase of initial denaturation at a temperature between 90° C. and 100° C., typically about 94° C., for a time of from 5 to 8 minutes; then a second phase of amplification comprising several cycles, typically 35 cycles, each cycle comprising 30 seconds at 94° C., then 30 seconds at 58° C., then 30 seconds at 72° C.; and a last phase of return to 72° C. for about 4 minutes. At the end of the PCR, the amplicons are stored, preferably at 4° C.

Typically, the primers usable in step e) of PCR are as follows:

| SEQ ID NO: | Primer |
|---|---|
| 596 | CMV Forward: |
| | CGC AAA TGG GCG GTA GGC GTG |
| 597 | CMV Reverse: |
| | CGC CAT CCA CGC TGT TTT G |
| 598 | pcDNA3 Forward: |
| | GGC TAA CTA GAG AAC CCA CTG |
| 599 | pcDNA3 Reverse: |
| | GGC AAC TAG AAG GCA CAG TC |
| 600 | pCEP Forward: |
| | AGA GCT CGT TTA GTG AAC CG |
| 601 | pCEP Reverse: |
| | GTG GTT TGT CCA AAC TCA TC |
| 602 | pEGFPC1 Forward: |
| | GAT CAC TCT CGG CAT GGA C |
| 603 | pEGFPC1 Reverse: |
| | CAT TTT ATG TTT CAG GTT CAG GG |
| 604 | pEGFPN1 Forward: |
| | GTC GTA ACA ACT CCG CCC |
| 605 | pEGFPN1 Reverse: |
| | GTC CAG CTC GAC CAG GAT G |
| 606 | pGex Forward: |
| | ATA GCA TGG CCT TTG CAG G |
| 607 | pGex Reverse: |
| | GAG CTG CAT GTG TCA GAG G |
| 608 | pGL Forward: |
| | GTA TCT TAT GGT ACT GTA ACT G |
| 609 | pGL Reverse: |
| | CTT TAT GTT TTT GGC GTC TTC C |
| 610 | pShuttleCMV Forward: |
| | GGT CTA TAT AAG CAG AGC TG |
| 611 | pShuttleCMV Reverse: |
| | GTG GTA TGG CTG ATT ATG ATC AG |
| 32 | Reverse: |
| | GGGTTCCCTAAGGGTTGGA |
| | (complementary to the priming sequence |
| | SEQ ID NO: 34) |
| 33 | Forward: |
| | GTGCCAGCAAGATCCAATCTAGA |

Preferably, the method according to the invention comprises a step 0 of analysis of the results of the PCR in step e), preferably by pyrosequencing. For this purpose, a pyrosequencer may be used, such as the PyroMark Q24 pyrosequencer sold by the company Qiagen, using the commercial kit Pyromark Gold Q24 Reagent and one of the priming oligonucleotides.

This analysis step allows immediate reading of the result, and indicates directly whether the sample from the subject bears a specific translocation, whether or not identified. This is notably demonstrated in FIG. 3.
In fact, in step f), if, for a biological sample from a subject, PCR amplification is obtained in step e) following hybridization to a pair of probes, then the subject has the cancer connected with the chromosome rearrangement corresponding to the pair of probes identified.

The various sequences mentioned in the present invention are summarized in the following table:

| SEQ ID NO: | Identification |
|---|---|
| 1-25, 30, 31 and 113 to 120 | Probes as such, notably for diagnosis of |
| | leukemias |
| 32 | Reverse PCR primer |
| 33 | Forward PCR primer and priming |
| | sequence at 5′ |
| 34 | Priming sequence at 3′ |
| 35-59, 64, 65 and 267-274 | Probes SEQ ID NO: 1-25, 30, 31 and 113 |
| | to 120 fused with the priming sequence |
| 26-29, 66-112 and 121-219 | Probes as such, notably for diagnosis of |
| | leukemias |
| 60-63, 220-266 and 275-373 | Probes SEQ ID NO: 26-29, 66-112 and |
| | 121-219 fused with the priming sequence |
| 374-405 | Probes as such, notably for diagnosis of |
| | sarcomas |
| 406-437 | Probes SEQ ID NO: 374-405 fused with |
| | the priming sequence |
| 438-480 | Probes as such, notably for diagnosis of |
| | solid tumors |
| 481-523 | Probes SEQ ID NO: 438-480 fused with |
| | the priming sequence |
| 524-559 | Probes as such, notably for diagnosis of |
| | carcinomas |
| 560-595 | Probes SEQ ID NO: 524-559 fused with |
| | the priming sequence |
| 596-611 | PCR primers |
| 612-615 | Sequences in FIG. 3A and B |
| 616-674 | Probes as such, notably for diagnosis of |
| | leukemias |
| 675-733 | Probes SEQ ID NO: 616-674 fused with |
| | the priming sequence |
| 734-741 | Probes as such, notably for diagnosis of |
| | carcinomas |
| 742-749 | Probes SEQ ID NO: 734-741 fused with |
| | the priming sequence |
| 750-774 | Probes as such, notably for diagnosis of |
| | sarcomas |
| 775-799 | Probes SEQ ID NO: 750-774 fused with |
| | the priming sequence |

FIG. 1: Scheme for definition of the probes.

FIG. 2: Scheme of RT-MLPA.

A) Example of translocation in the intron located between exon 2 of gene 1 and exon 2 of gene 2, leading to a fusion mRNA.

B) Step 1: reverse transcription of this fusion mRNA, to obtain a cDNA.

C) Step 2: incubation with the probes and hybridization of the latter to the complementary portions of cDNA. Probe 51 consists of a complementary sequence of the last nucleotides of exon 2 of gene 1 of cDNA, and probe S2 consists of a sequence complementary to the first nucleotides of exon 2 of gene 2 of cDNA.

Probe S1 is fused at 5′ with a priming sequence SA.

Probe S2 is fused at 3′ with a priming sequence SB.

Owing to the contiguity between exons 2 of gene 1 and of gene 2, probes 51 and S2 are side by side.

D) Step 3: ligation by a DNA ligase. The probes that are side by side are then bound. S1 and S2 thus form a continuous sequence, with SA and SB.

E) Step 4: PCR: using suitable primers, the bound probes are amplified. In this case, the primers used are the sequence SA, and the sequence complementary to SB (called B′).

F: analysis of the results obtained. The figure shows an example of results obtained with the probes that bind to the translocations involving the MLL gene (characteristic of leukemias) on samples obtained from 15 patients (tracks 1 to 15). Tracks 16 and 17 are negative controls.

It can be seen that 11 tumors out of the 15 have a rearrangement of the MLL gene.

FIG. 3: Identification of the translocations in leukemia cases. Identification of the partners of the MLL gene of samples No. 14 (A) and 15 (B) in FIG. 2. The PCR amplification products were analyzed by pyrosequencing.

Sequence A corresponds to a junction between the 9th exon of the MLL gene and the 2nd exon of the AF6 gene, indicating that the tumor had a translocation t(6;11)(q27;q23).

Sequence B corresponds to a junction between the 10th exon of the MLL gene and the 2nd exon of the AF1Q gene, indicating that the tumor had a translocation t(1;11)(q21;q23).

These two translocations confirm the diagnosis of acute leukemia, and are associated with forms with a very poor prognosis.

FIG. 4: Table of probes SEQ ID NO: 1 to 25, 30, 31 and 113 to 120 (corresponding to the probes fused with the priming sequence SEQ ID NO: 35 to 59, 64, 65 and 267 to 274).

The table shows the gene and the exon to which the probe hybridizes. The column “SEQ ID NO:” gives the sequence number of the probe as such, and in parentheses the sequence number of the probe fused with the priming sequence.

These probes are usable notably for diagnosis of leukemias.

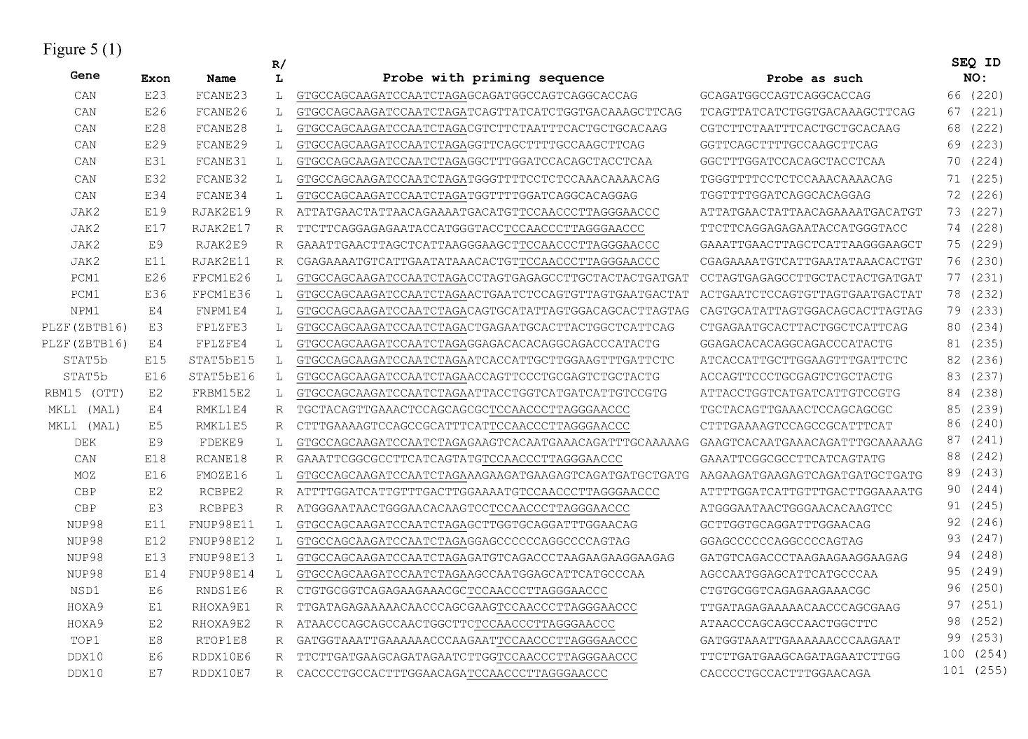

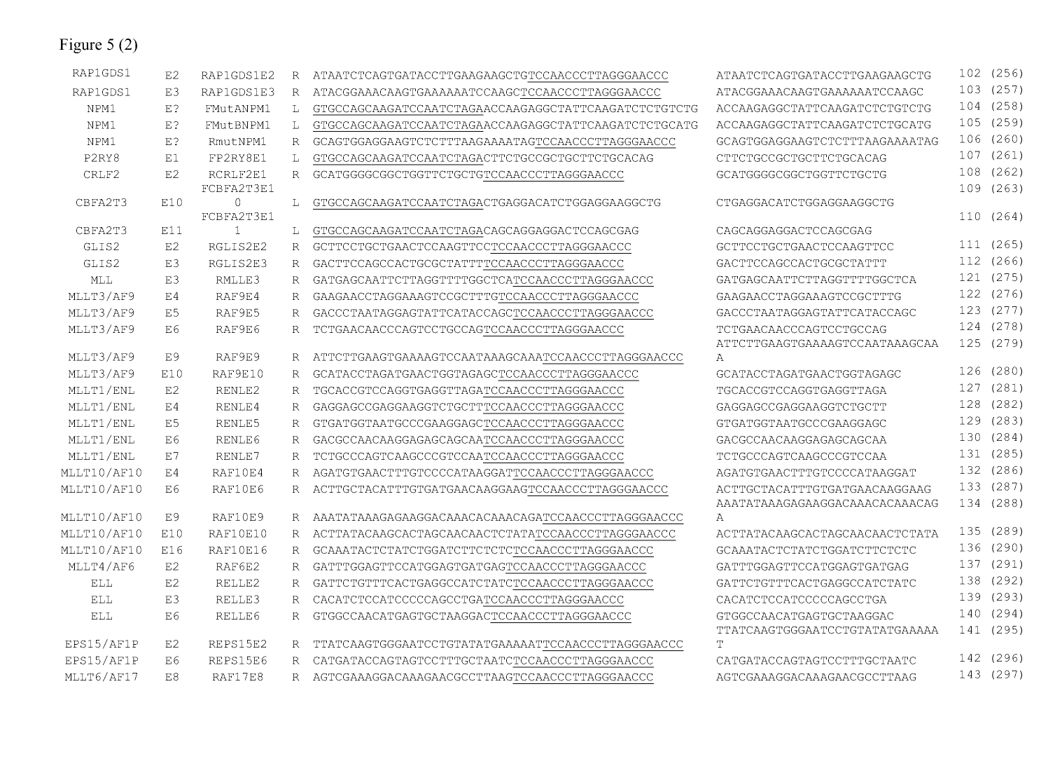

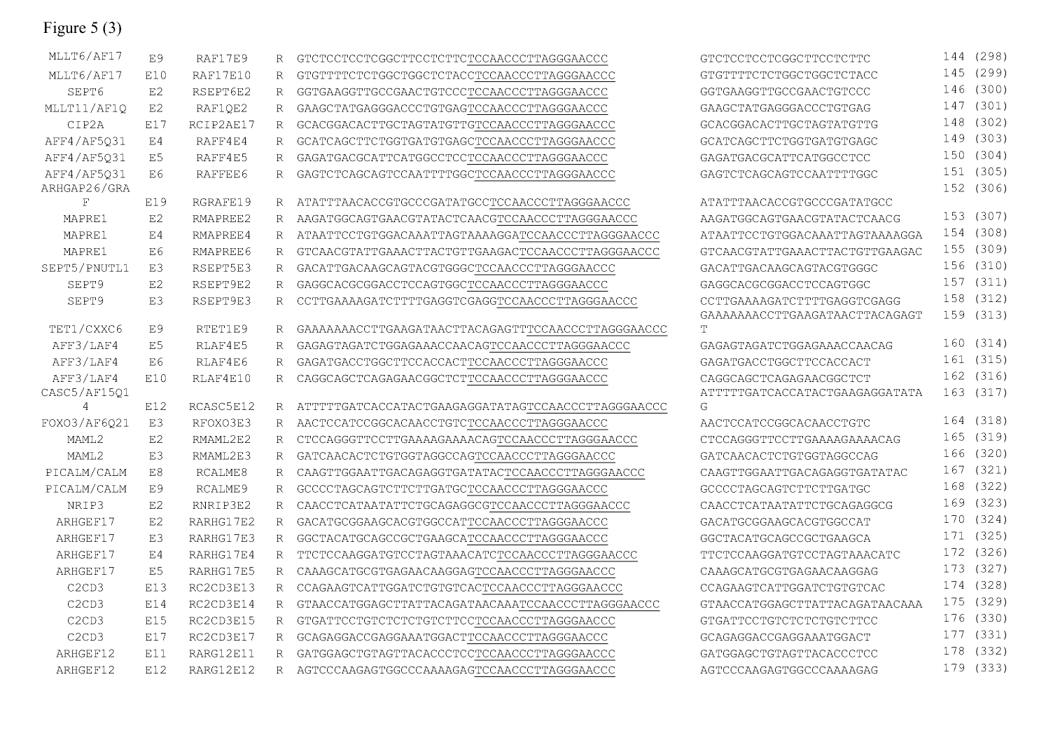

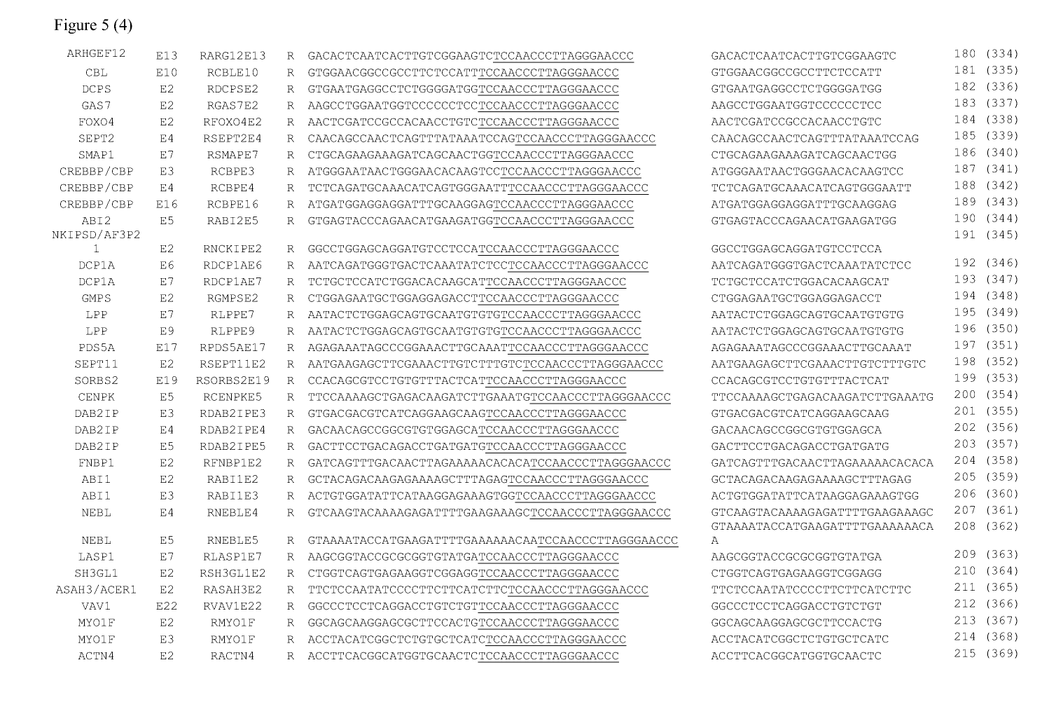

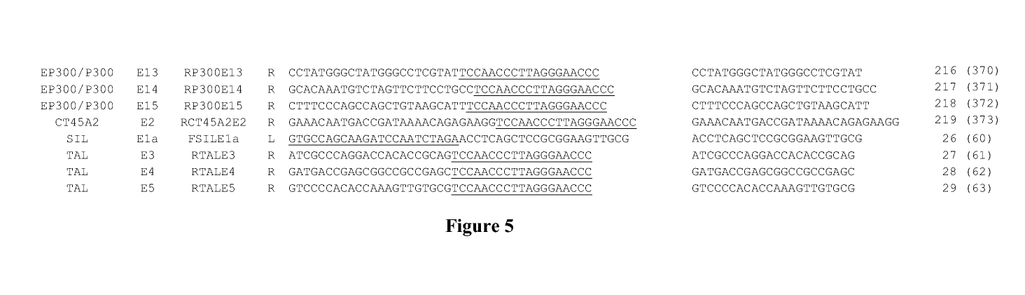

FIG. 5: Table of probes SEQ ID NO: 26 to 29, 66 to 112 and 121 to 219 (corresponding to the probes fused with the priming sequence SEQ ID NO: 60 to 63, 220 to 266 and 275 to 373).

The table shows the gene and the exon to which the probe hybridizes. The column “SEQ ID NO:” gives the sequence number of the probe as such, and in parentheses the sequence number of the probe fused with the priming sequence.

These probes are usable notably for diagnosis of leukemias.

FIG. 6: Identification of the translocations in cases of leukemia (A) and of solid tumors (B).

The PCR amplification products were analyzed by pyrosequencing.

A) The translocations identified correspond to:

“AML1 exon5-ETO exon2”: AML2 (type 2 Acute myeloblastic leukemia)

“MLL exon9-AF10 exon9”: ALL (Acute lymphoblastic leukemia)

“BCR exon13-ABL exon2”: chronic myeloid leukemia

“PML exon3-RARa exon3”: AML3 (type 3 Acute myeloblastic leukemia)

“BCR exon1-ABL exon2”: ALL (Acute lymphoblastic leukemia); and

“CBFB exon5-MYH11 exon12”: AML4 (type 4 Acute myeloblastic leukemia).

B) The translocations identified correspond to a synovial sarcoma (SYT-SSX), to a rhabdomyosarcoma (PAX-FKHR), and to an anaplastic lymphoma (NPM-ALK).

The term “Tneg” denotes a control sample, obtained from a known tumor that does not have the genetic rearrangements being assayed.

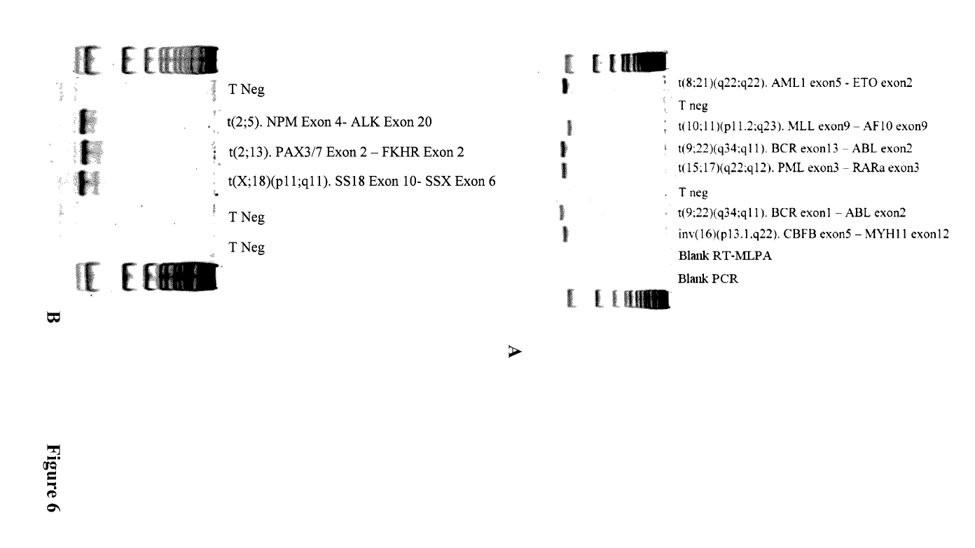

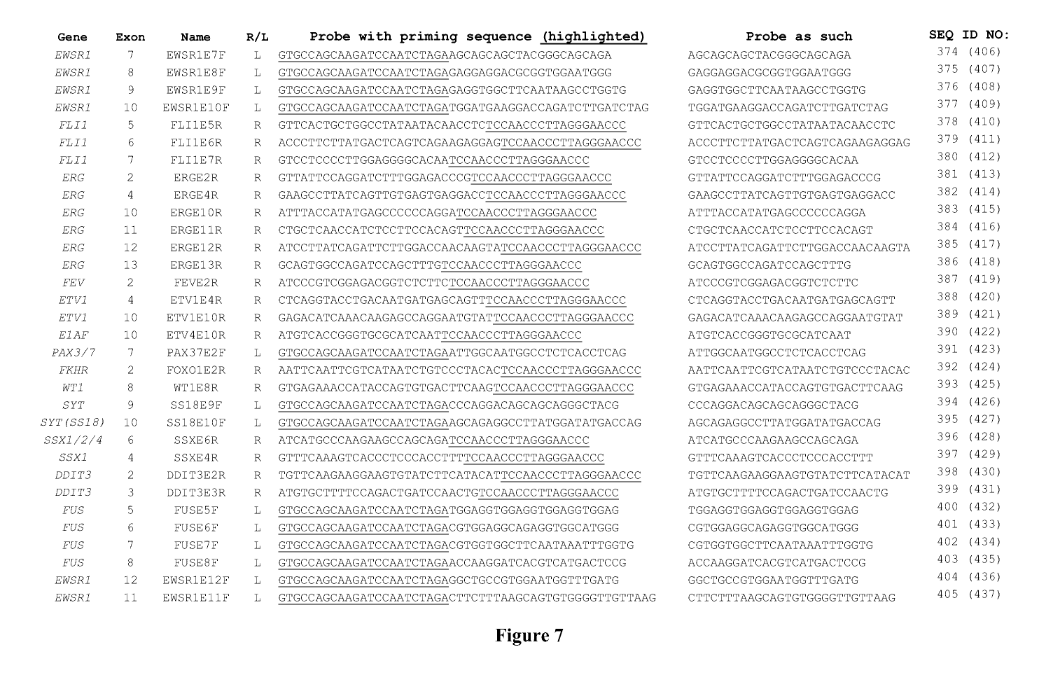

FIG. 7: Table of probes SEQ ID NO: 374 to 405 (corresponding to the probes fused with the priming sequence SEQ ID NO: 406 to 437).

The table shows the gene and the exon to which the probe hybridizes. The column “SEQ ID NO:” gives the sequence number of the probe as such, and in parentheses the sequence number of the probe fused with the priming sequence.

These probes are usable notably for diagnosis of sarcomas.

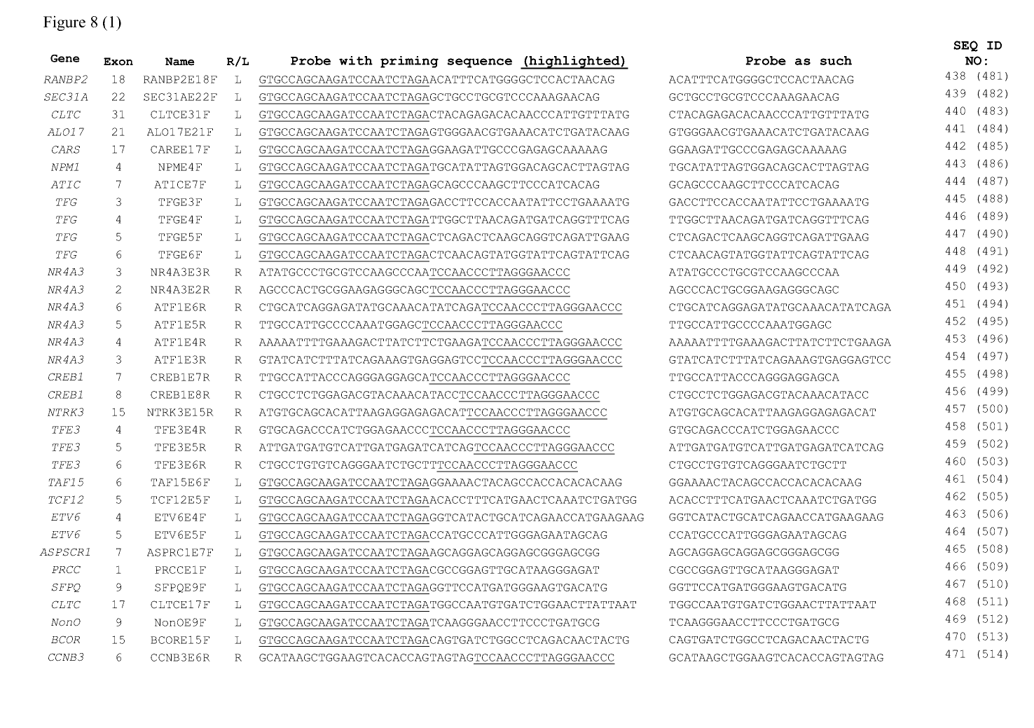

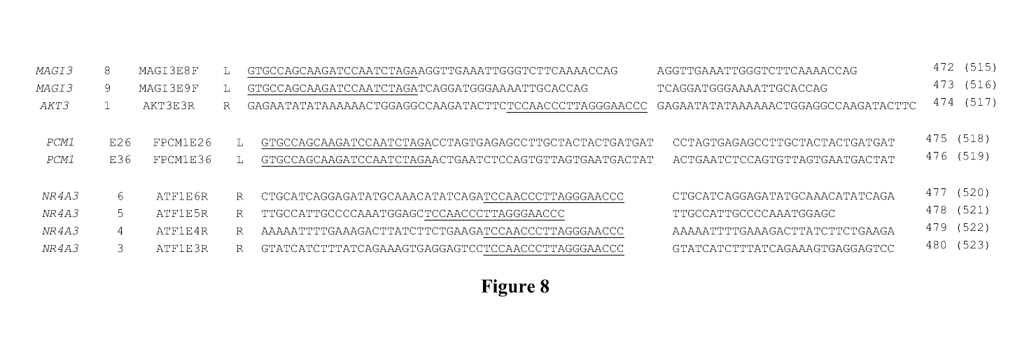

FIG. 8: Table of probes SEQ ID NO: 438 to 480 (corresponding to the probes fused with the priming sequence SEQ ID NO: 481 to 523).

The table shows the gene and the exon to which the probe hybridizes. The column “SEQ ID NO:” gives the sequence number of the probe as such, and in parentheses the sequence number of the probe fused with the priming sequence.

These probes are usable notably for diagnosis of sarcomas.

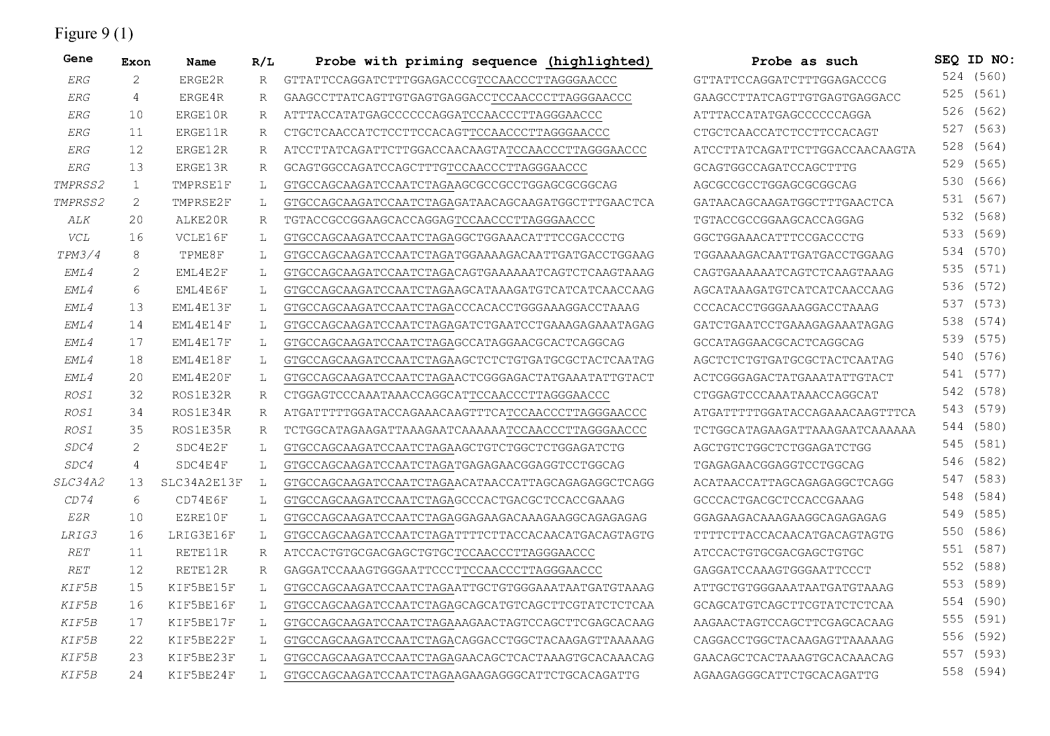

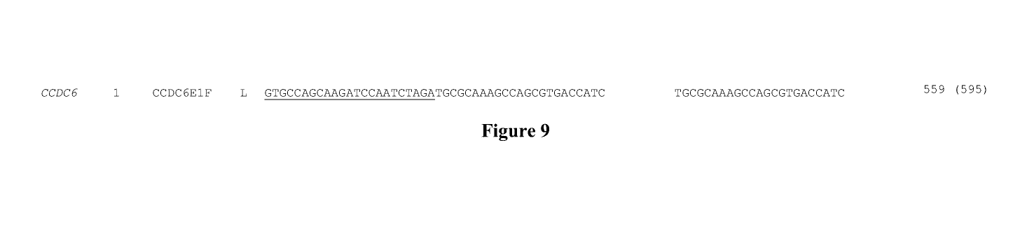

FIG. 9: Table of probes SEQ ID NO: 524 to 559 (corresponding to the probes fused with the priming sequence SEQ ID NO: 560 to 595).

The table shows the gene and the exon to which the probe hybridizes. The column “SEQ ID NO:” gives the sequence number of the probe as such, and in parentheses the sequence number of the probe fused with the priming sequence.

These probes are usable notably for diagnosis of carcinomas.

FIG. 10: Identification of the fusion genes.

A) Fusion genes in leukemia cases. The fusion transcripts hybridize to the probes SEQ ID NO: 1 to 25, 30, 31 and 113 to 120.

B) Fusion genes in sarcoma cases. The fusion transcripts hybridize to the probes SEQ ID NO: 374 to 405.

C) Fusion genes in carcinoma cases. The fusion transcripts hybridize to the probes SEQ ID NO: 524 to 559.

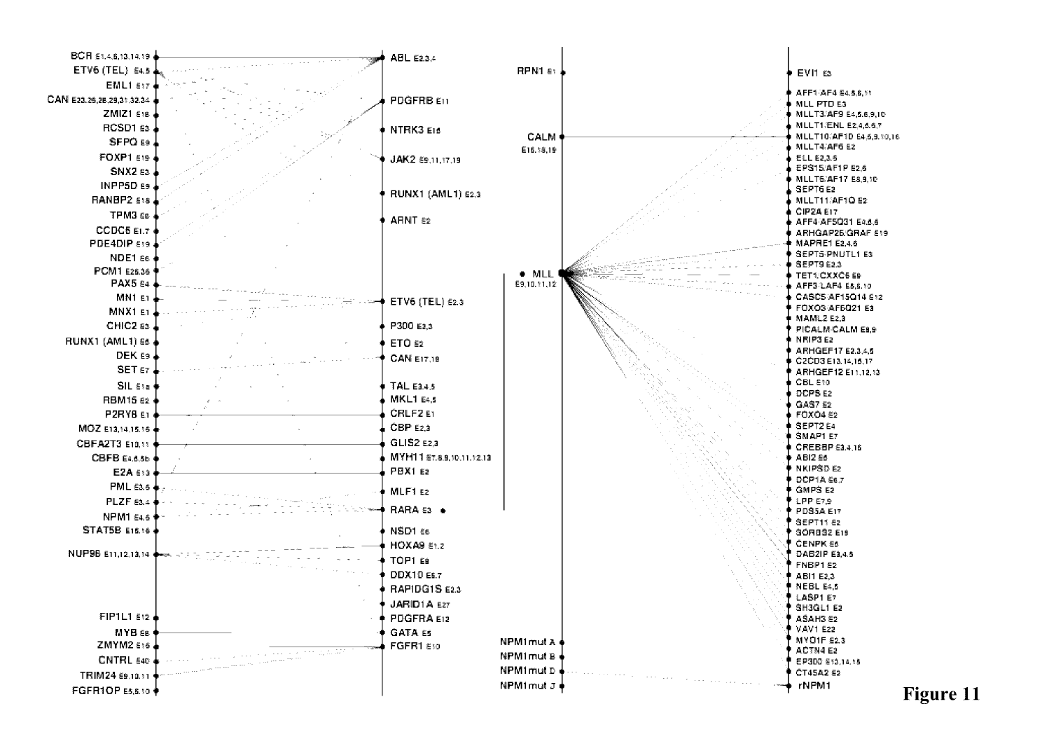

FIG. 11: Identification of the fusion genes in the case of leukemias.

The use of probes SEQ ID NO: 1 to 31, 66 to 219, 438, 467, 534, 559, 616 to 639, 641 to 670, 737 and 759 in a single kit would allow simultaneous searching for more than 110 different gene rearrangements in leukemias.

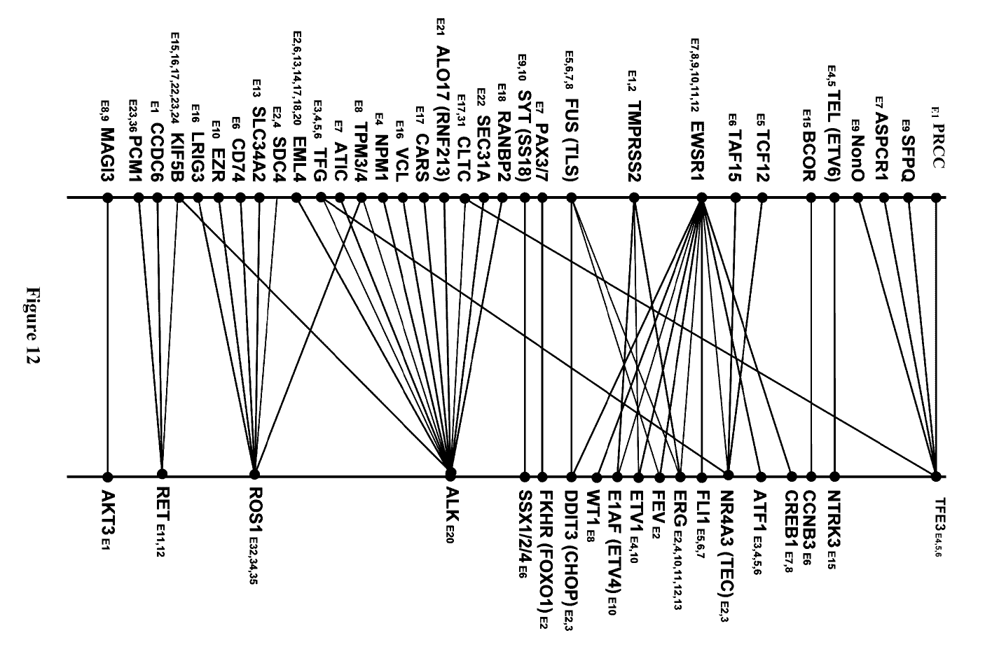

FIG. 12: Identification of the fusion genes in the case of sarcomas and carcinomas.

The fusion transcripts hybridize to the probes SEQ ID NO: 374 to 405, 524 to 559 and 438 to 480.

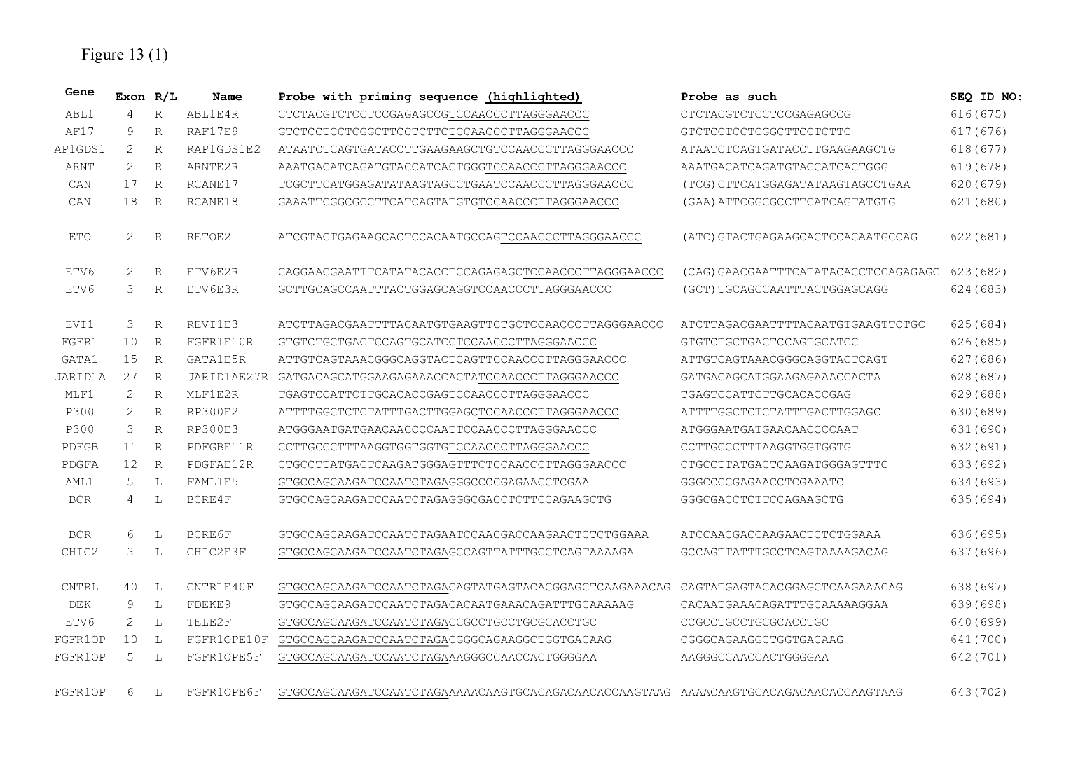

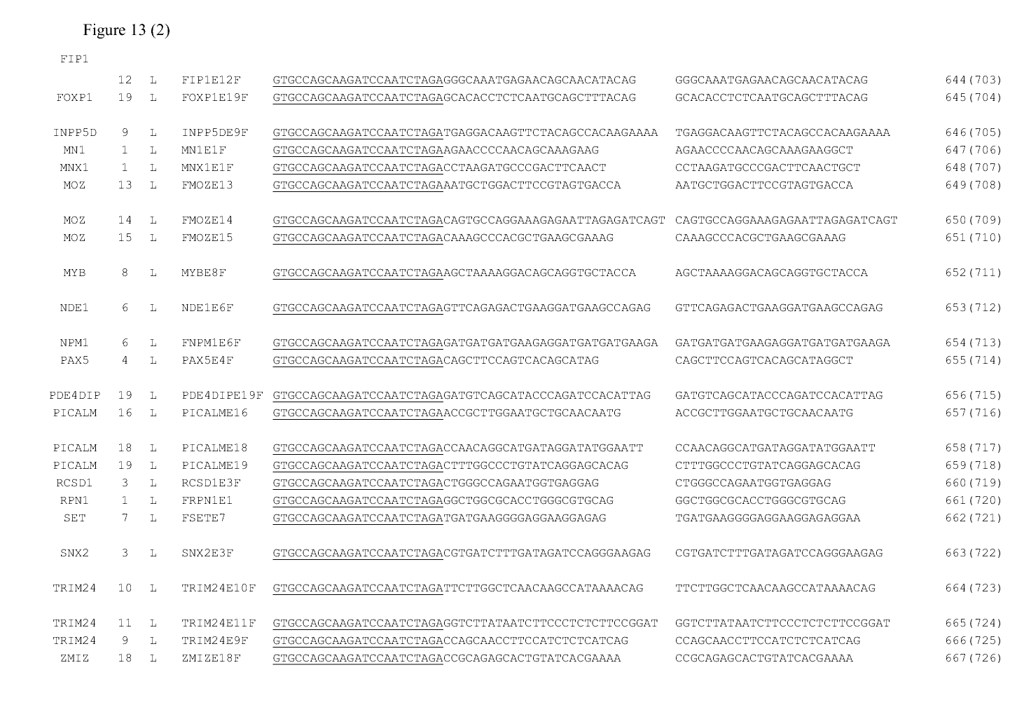

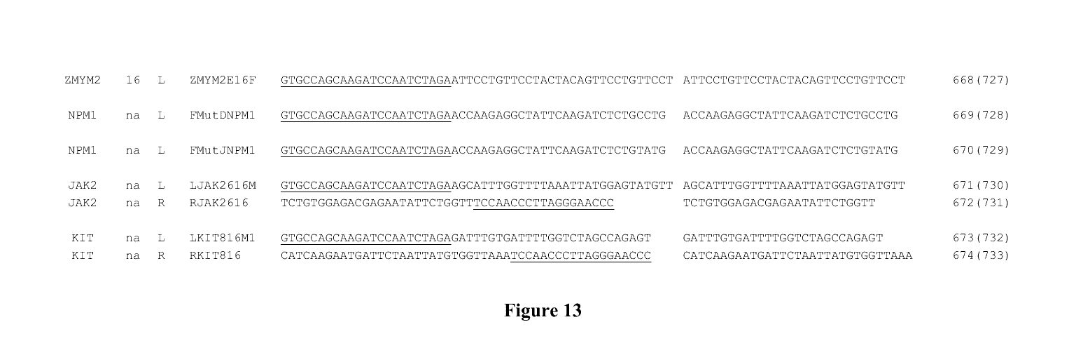

FIG. 13: Table of probes SEQ ID NO: 616-674 (corresponding to the probes fused with the priming sequence SEQ ID NO: 675-733).

The table shows the gene and the exon to which the probe hybridizes. The column “SEQ ID NO:” gives the sequence number of the probe as such, and in parentheses the sequence number of the probe fused with the priming sequence.

These probes are usable notably for diagnosis of leukemias.

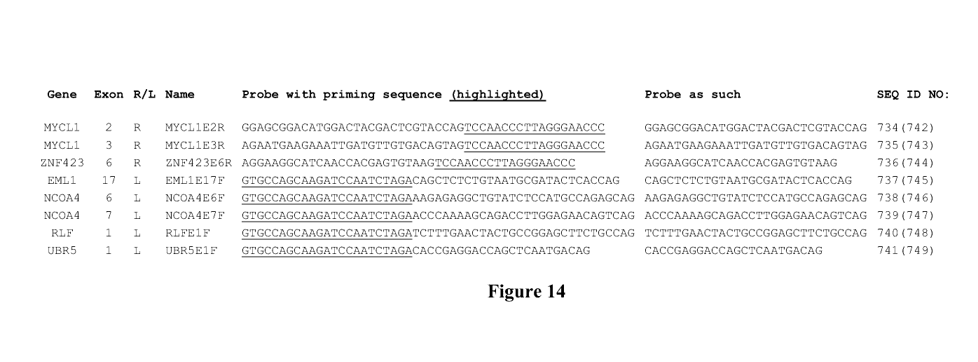

FIG. 14: Table of probes SEQ ID NO: 734-741 (corresponding to the probes fused with the priming sequence SEQ ID NO: 742-749).

The table shows the gene and the exon to which the probe hybridizes. The column “SEQ ID NO:” gives the sequence number of the probe as such, and in parentheses the sequence number of the probe fused with the priming sequence.

These probes are usable notably for diagnosis of carcinomas.

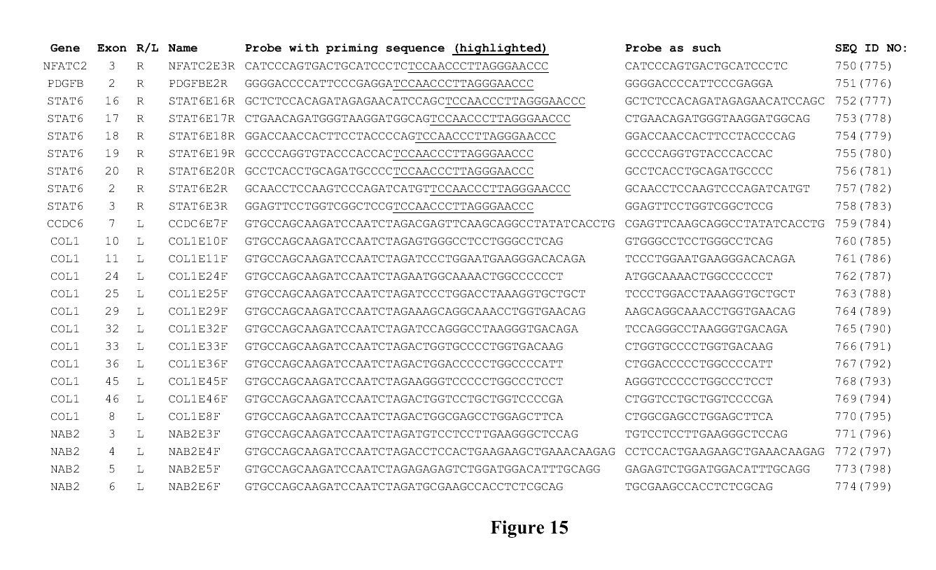

FIG. 15: Table of probes SEQ ID NO: 750-774 (corresponding to the probes fused with the priming sequence SEQ ID NO: 775-799).

The table shows the gene and the exon to which the probe hybridizes. The column “SEQ ID NO:” gives the sequence number of the probe as such, and in parentheses the sequence number of the probe fused with the priming sequence.

These probes are usable notably for diagnosis of carcinomas.

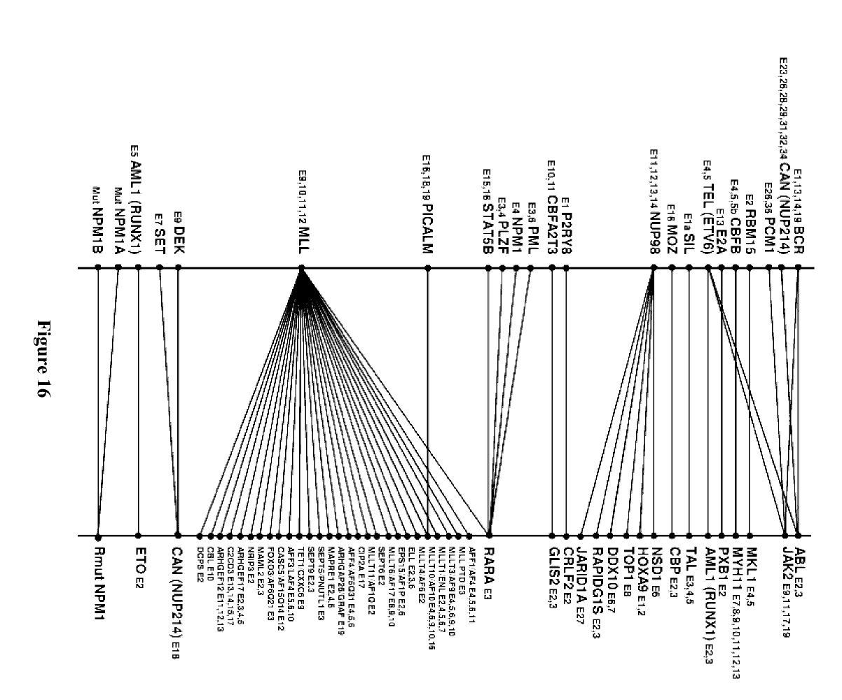

FIG. 16: Identification of the fusion genes targeted in example 2.

The fusion transcripts hybridize to the probes SEQ ID NO: 1 to 31, 66 to 167, 169 to 182, 628, 657 to 659 and 662.

### EXAMPLE 1

### Material and Methods

Step a): extraction of RNA:

The cells obtained from blood or from bone marrow or from biopsy fragments are stored at −80° C. in 1 mL of trizol (Life Technologies, Carlsbad, Calif., USA). 200 μl of chloroform (Merck, Darmstadt, Germany) is added to each previously thawed sample.

The mixture is homogenized before being incubated for about 5 min in ice and then centrifuged for 15 min at 12000 rpm and 4° C.

500 μl of 100% isopropanol (Sigma Aldrich, St Quentin Fallavier, France) is added to the previously isolated aqueous phase.

The mixture is homogenized before being incubated for about 10 min in ice and centrifuged for 10 min at 12000 rpm and 4° C.

After decanting, the sediments are washed with about 1 mL of 70% ethanol and centrifuged for 5 min at 7500 rpm and 4° C.

After another 2 washing operations the sediments are decanted and dried before being taken up in about 100 μl of water.

The RNAs are then incubated on a water bath at about 55° C. for 5 to 10 min.

The concentration of the RNAs obtained is adjusted to between 50 and 250 ng/μL.

Step b): reverse transcription:

4 μl RNA (200-1000 ng) is incubated with 2.5 μl Buffer 5× (Thermo Fisher Scientific, Waltham, Mass., USA), 1 μl DTT 100 mM (Thermo Fisher Scientific, Waltham, Mass., USA), 2 μl dNTPs 10 mM (Thermo Fisher Scientific, Waltham, Mass., USA) and 2 μl of hexamers at 100 pmol/μl (Thermo Fisher Scientific, Waltham, Mass., USA), for 2 minutes at 80° C., then 5 minutes at 37° C., and then the samples are stored at 4° C.

Reverse transcriptase (RT) is then added (1 μL RT (30u) (M-MLV, Thermo Fisher Scientific, Waltham, Mass., USA)), and the resultant mixture is incubated for 15 minutes at 37° C., then 2 minutes at 98° C., and then stored at 4° C.

Step c): incubation and hybridization of the cDNA to the probes (MLPA):

5 μl of the preceding mixture (˜half of the initial reaction mixture) obtained in step b) is incubated with 1.5 μL MLPA Buffer (Old Salsa MLPA Buffer. MRC Holland, Amsterdam, the Netherlands) and 1.5 μL of the mixture of probes described below, for 2 minutes at 95° C., and then at least 1 hour at 60° C.

Mixture of Probes for MLPA (3 fmol/1.5 μl Final):

First step: Taking up each probe with 100 μM (H₂O)

Second step: Dilution of each probe with 10 μM (H₂O)

Third step: Mixture of probes: 2 μl of each probe at 10 μM

Final volume: +volume idem TE20:2 for Mix to final TE10:1

Fourth step: Final dilution: (0.2 μl of the mixture of probes F and R from step 3× in probes)+TE10:1 q.s. 1 ml

(example: mixture of probes F with 10 different probes: 0.2×10=2 μl mixture F; mixture R with 10 probes: 0.×10=4 μl mixture R; Final Mixture=2 μl mixture F+4 μl mixture R+994 μl TE10:1)

Step d): Ligation:

Ligase 65 (n tubes+10%) (Salsa Ligase-65. MRC-Holland, Amsterdam, the Netherlands) is prepared with the following mixture:

3 μL Ligase Buffer A (Ligase Buffer A, MRC-Holland, Amsterdam, the Netherlands),

3 μL Ligase Buffer B (Ligase Buffer B, MRC-Holland, Amsterdam, the Netherlands), 254 H₂O

Vortex

1 μL ligase 65 mix (Salsa Ligase-65. MRC-Holland, Amsterdam, the Netherlands) Vortex.

The mixture of probes from step c) is mixed at 54° C. with 32 μL of this ligase mix, then incubated for 15 minutes at 54° C., and 5 minutes at 98° C., and then stored at 4° C.

Step e): PCR:

PCR (n tubes+10%) is carried out in the following mixture:

20 μl of Eurogentec Red‘y’Start Mix (Eurogentec, Angers, France)

1 μl of primer SEQ ID NO: 32 (Biot)

1 μl of primer SEQ ID NO: 33

13 μl H₂O

5 μl of the mixture from step d) is mixed with 35 μl of the above PCR mixture, and is then submitted to the following PCR programme:

| | | |
|---|---|---|
| 35x | 94° C. | 6 min |
| | 94° C. | 30 s |
| | 58° C. | 30 s |
| | 72° C. | 30 s |
| | 72° C. | 4 min |
| | 4° C. | |

Step f): Pyrosequencing:

Control (optional): 8% acrylamide gel (Acrylamide/Bis-Acrylamide 29:1, 40%, Biosolve B.V., Valkenswaard, the Netherlands)

20 μl of the product amplified by PCR is analyzed by pyrosequencer.

### Results

The results obtained are presented in FIGS. 2, 3 and 6A for the leukemia cases, and in FIG. 6B for the solid tumors.

At the end of PCR, if the translocation that is being sought is detected, the sample is positive, and the diagnosis of the disease is immediate.

### EXAMPLE 2

In acute leukemia, recurrent chromosome translocations that lead to fusion of two genes are frequent. Certain of these markers have a well established prognostic and therapeutic impact and are systematically monitored at the time of diagnosis by cytogenetics and RT-PCR. However, owing to the limitations of these methods, only a few known rearrangements among all those that exist are systematically assayed. A great many abnormalities that could supply important clinical information are thus ignored, mainly because it is impossible to perform cost-effective, quick and reliable multi-target screening. The assay proposed here is simple and allows reliable detection of dozens of fusion genes in just a few hours.

The assay has been designed for simultaneous detection of more than 50 translocations involving 70 recurrent genes in acute myeloblastic leukemias (AML), acute lymphoblastic leukemias (ALL) and chronic myeloid leukemias (CML).

Samples of cDNA obtained from leukemic cells are first incubated with a mixture of oligonucleotide probes that are complementary to the ends of the exons, at the abnormal junctions on the fusion mRNAs. These probes correspond to the fusion transcripts listed in FIG. 16, and have the sequence SEQ ID NO: 1 to 31, 66 to 167, 169 to 182, 628, 657 to 659 and 662. For most of the genes, different probes have been designed for detecting different transcripts resulting from alternative genomic recombinations (for example on exons 1, 13, 14 and 19 for the BCR gene, and on exons 2 and 3 for the ABL gene). The mixture thus combines more than 150 probes and is aimed at more than 400 different fusion transcripts. All the probes on the left have a common tail (S_{A}) at their 5′ end, and all the probes on the right have a common tail (S_{B}) at their 3′ end (cf. FIGS. 2A to E).

Additional probes have also been included for detecting the most frequent mutations of the NPM1 gene (A, B, D). If a translocation is present in the sample, the two probes hybridize to one another side by side on the fusion cDNA. A DNA ligase is then used for creating a covalent bond between these probes, which allows them to be amplified by PCR with the primers S_{A} and S_{B}. If a PCR product is amplified, the two partners are identified by sequence analysis.

This method was applied to a retrospective series of 430 patients (252 AML and 178 children with ALL). In the ALL-B (147 cases), the 33 rearrangements ETV6-RUNX1, the 6 rearrangements BCR ABL and the 5 rearrangements TCF3-PBX1, as well as the 6 rearrangements MLL (3 AF4; 1 ENL, 1 AF9 and 1 AFF4) identified at the time of diagnosis by conventional methods, were detected, as well as 5 previously unknown junctions P2RY8-CRLF2.

In the ALL-T (31 cases), 6 known rearrangements (4 SIL-TAL, 2 CALM-AF10) and 5 fusions not detected previously (2 NUP214-ABL, 1 MLL-ENL, 1 ETV6-ABL, and a new junction PLZF-ABL) were detected.

In the AMLs, 86 fusions were detected: 23 PML-RARA, 2 PLZF-RARA, 18 CBFB-MYH11, 12 RUNX1-RUNX1T1, 4 NUP98-NSD1, 2 BCR-ABL, 1 DEK-NUP214, 1 CALM-AF10, 1 MOZ-CBP, 22 rearrangements MLL (13 PTD, 3 AF9, 2 AF6, 1 AF10, 1 ENL, 1 AF1Q and 1 MAPRE) and 44 mutations of NPM1. In particular, 20 translocations of this series, including 14 fusions of the MLL gene and one cryptic cytogenetic abnormality t(8, 21) had not been identified in the diagnosis.

Moreover, all these new abnormalities (in the AMLs and ALLs) could be confirmed by conventional RT-PCR and sequencing, demonstrating the specificity of the method.

In the whole cohort of 430 patients, the three methods thus detected 157 fusions. 85 fusions (54.1%) and 112 fusions (71.3%) were detected at the time of diagnosis by cytogenetics or by RT-PCR respectively, and 152 fusions (96.8%) were detected by the present method.

In conclusion, the method according to the invention is a simple multiplex assay that can reveal a very large number of recurrent gene fusions in leukemia. Its short turnaround (up to 40 patients can be tested in parallel and the results can be obtained in less than a day) and its low cost (just PCR apparatus, a pyrosequencer and basic reagents for molecular biology are required) make it particularly suitable for everyday practice. Its ability to detect a great many abnormalities that are hardly ever tested in everyday practice could supply many diagnoses and prognoses, and allow stratification of patients in prospective clinical trials.

1. A method for diagnosing a cancer in a subject, comprising a step of RT-MLPA on a biological sample obtained from said subject using at least one pair of probes selected from:
probes SEQ ID NO: 1 to 25, 30, 31 and 113 to 120,
probes SEQ ID NO: 374 to 405, and
probes SEQ ID NO: 524 to 559,
each of the probes being fused, at one end at least, with a priming sequence.

2. The method as claimed in claim 1, in which the step of RT-MLPA on a biological sample obtained from the subject is carried out with at least the probes SEQ ID NO: 1 to 25, 30, 31 and 113 to 120, and/or with at least the probes SEQ ID NO: 374 to 405, and/or with at least the probes SEQ ID NO: 524 to 559, each of the probes being fused, at one end at least, with a priming sequence.

3. The method as claimed in claim 1, wherein the priming sequence is selected from the sequences SEQ ID NO: 33 and SEQ ID NO: 34.

4. The method as claimed in claim 1, wherein said biological sample is selected from whole blood, bone marrow and a biopsy from said subject.

5. The method as claimed in claim 1, wherein the probes SEQ ID NO: 26 to 29, 66 to 112 and 121 to 219 and/or the probes SEQ ID NO: 616 to 674 are also used for the RT-MLPA step, each of the probes being fused, at one end at least, with a priming sequence.

6. The method as claimed in claim 1, wherein the probes SEQ ID NO: 750 to 774 are also used for the RT-MLPA step, each of the probes being fused, at one end at least, with a priming sequence.

7. The method as claimed in claim 1, wherein the probes SEQ ID NO: 734 to 741 are also used for the RT-MLPA step, each of the probes being fused, at one end at least, with a priming sequence.

8. The method as claimed in claim 1, wherein the RT-MLPA step comprises at least the following steps:
a) extraction of RNA from the biological sample from the subject;
b) conversion of the RNA extracted in a) to cDNA by reverse transcription;
c) incubation of the cDNA obtained in b) with at least one pair of probes selected from:
probes SEQ ID NO: 1 to 25, 30, 31 and 113 to 120,
probes SEQ ID NO: 374 to 405, and
probes SEQ ID NO: 524 to 559,
each of the probes being fused, at one end at least, with a priming sequence,
preferably with at least the probes SEQ ID NO: 1 to 25, 30, 31 and 113 to 120, and/or with at least the probes SEQ ID NO: 374 to 405, and/or with at least the probes SEQ ID NO: 524 to 559, each of the probes being fused, at one end at least, with a priming sequence;
d) addition of a DNA ligase to the mixture obtained in c), in order to establish a covalent bond between two contiguous probes;
e) PCR amplification of the covalently bound contiguous probes obtained in d).

9. The method as claimed in claim 8, further comprising a step f) of analysis of the results of the PCR in step e), preferably by pyrosequencing.

10. The method as claimed in claim 9, wherein, in step f), if, for a biological sample from a subject, PCR amplification is obtained in step e) following hybridization to a pair of probes, then the subject has the cancer connected with the chromosome rearrangement corresponding to the pair of probes identified.

11. The method as claimed in claim 8, wherein the PCR amplification in step e) is carried out using the primers SEQ ID NO: 32 and 33.

12. The method as claimed in claim 1, which is a method for diagnosing a leukemia in a subject, comprising a step of RT-MLPA on a biological sample obtained from said subject with at least the probes SEQ ID NO: 1 to 25, 30, 31 and 113 to 120, each of the probes being fused, at one end at least, with a priming sequence; preferably with at least the probes SEQ ID NO: 35 to 59, 64, 65 and 267 to 274.

13. The method as claimed in claim 12, wherein the step of RT-MLPA on a biological sample obtained from the subject is carried out in addition with at least the probes SEQ ID NO: 60 to 63, 220 to 266 and 275 to 373, and/or the probes SEQ ID NO: 675 to 733.

14. The method as claimed in claim 1, which is a method for diagnosing a sarcoma in a subject, comprising a step of RT-MLPA on a biological sample obtained from said subject with at least the probes SEQ ID NO: 374 to 405, each of the probes being fused, at one end at least, with a priming sequence; preferably with at least the probes SEQ ID NO: 406 to 437.

15. The method as claimed in claim 14, wherein the step of RT-MLPA on a biological sample obtained from the subject is carried out in addition with at least the probes SEQ ID NO: 481 to 523 and/or the probes SEQ ID NO: 775 to 799.

16. The method as claimed in claim 1, which is a method for diagnosing a carcinoma in a subject, comprising a step of RT-MLPA on a biological sample obtained from said subject with at least the probes SEQ ID NO: 524 to 559, each of the probes being fused, at one end at least, with a priming sequence; preferably with at least the probes SEQ ID NO: 560 to 595.

17. The method as claimed in claim 16, wherein the step of RT-MLPA on a biological sample obtained from the subject is carried out in addition with at least the probes SEQ ID NO: 481 to 523 and/or the probes SEQ ID NO: 742 to 749.

18. A kit comprising at least the probes SEQ ID NO: 1 to 25, 30, 31 and 113 to 120, and/or the probes SEQ ID NO: 374 to 405, and/or the probes SEQ ID NO: 524 to 559, preferably further comprising the probes SEQ ID NO: 26 to 29, 66 to 112 and 121 to 219, and/or the probes SEQ ID NO: 616 to 674, and/or the probes SEQ ID NO: 438 to 480, and/or the probes SEQ ID NO: 750 to 774, and/or the probes SEQ ID NO: 734 to 741, each of the probes being fused, at one end at least, with a priming sequence.

19. A kit comprising at least the probes SEQ ID NO: 35 to 59, 64, 65 and 267 to 274, and/or the probes SEQ ID NO: 406 to 437, and/or the probes SEQ ID NO: 560 to 595, preferably further comprising the probes SEQ ID NO: 60 to 63, 220 to 266 and 275 to 373, and/or the probes SEQ ID NO: 675 to 733, and/or the probes SEQ ID NO: 775 to 799, and/or the probes SEQ ID NO: 742 to 749.


SEQUENCE LISTING (799 sequences)

SEQ ID NO: 1 - DNA, 22 bp, Artificial Sequence
  primer
aagcccttcagcggccagtagc

SEQ ID NO: 2 - DNA, 23 bp, Artificial Sequence
  primer
gtgaaaagctccgggtcttaggc

SEQ ID NO: 3 - DNA, 22 bp, Artificial Sequence
  primer
ggcgccttccatggagacgcag

SEQ ID NO: 4 - DNA, 25 bp, Artificial Sequence
  primer
attccgctgaccatcaataaggaag

SEQ ID NO: 5 - DNA, 26 bp, Artificial Sequence
  primer
agccactggatttaagcagagttcaa

SEQ ID NO: 6 - DNA, 23 bp, Artificial Sequence
  primer
actgaaggcagccttcgacgtca

SEQ ID NO: 7 - DNA, 24 bp, Artificial Sequence
  primer
gctcttgcatcacccaggggaaag

SEQ ID NO: 8 - DNA, 20 bp, Artificial Sequence
  primer
cagtggcgccggggaggcag

SEQ ID NO: 9 - DNA, 25 bp, Artificial Sequence
  primer
ccattgagacccagagcagcagttc

SEQ ID NO: 10 - DNA, 24 bp, Artificial Sequence
  primer
gagtttgatgaggagcgagcccag

SEQ ID NO: 11 - DNA, 25 bp, Artificial Sequence
  primer
caggtctcatcgggaggaaatggag

SEQ ID NO: 12 - DNA, 26 bp, Artificial Sequence
  primer
agtttcacagctgctggcagtaactg

SEQ ID NO: 13 - DNA, 27 bp, Artificial Sequence
  primer
gccaaggcgaacctagacaagaataag

SEQ ID NO: 14 - DNA, 26 bp, Artificial Sequence
  primer
aatgaagttgagagcgtcacagggat

SEQ ID NO: 15 - DNA, 25 bp, Artificial Sequence
  primer
gagctgcttcaagaagaaacccggc

SEQ ID NO: 16 - DNA, 25 bp, Artificial Sequence
  primer
ctctccgactcgaagaagaagctgc

SEQ ID NO: 17 - DNA, 29 bp, Artificial Sequence
  primer
ttgttagccgaggagaaaaacatctcttc

SEQ ID NO: 18 - DNA, 25 bp, Artificial Sequence
  primer
gtccatgagctggagaagtccaagc

SEQ ID NO: 19 - DNA, 28 bp, Artificial Sequence
  primer
cttcacgagtatgagacggaactggaag

SEQ ID NO: 20 - DNA, 25 bp, Artificial Sequence
  primer
tctcggcctcccgactcctacagtg

SEQ ID NO: 21 - DNA, 26 bp, Artificial Sequence
  primer
ttttgagtatccgaggagcccaggag

SEQ ID NO: 22 - DNA, 29 bp, Artificial Sequence
  primer
ggtcatactgcatcagaaccatgaagaag

SEQ ID NO: 23 - DNA, 24 bp, Artificial Sequence
  primer
ccatgcccattgggagaatagcag

SEQ ID NO: 24 - DNA, 30 bp, Artificial Sequence
  primer
aatgcatacttggaatgaatccttctagag

SEQ ID NO: 25 - DNA, 23 bp, Artificial Sequence
  primer
atgccagcacgagccgccgcttc

SEQ ID NO: 26 - DNA, 23 bp, Artificial Sequence
  primer
acctcagctccgcggaagttgcg

SEQ ID NO: 27 - DNA, 22 bp, Artificial Sequence
  primer
atcgcccaggaccacaccgcag

SEQ ID NO: 28 - DNA, 22 bp, Artificial Sequence
  primer
gatgaccgagcggccgccgagc

SEQ ID NO: 29 - DNA, 22 bp, Artificial Sequence
  primer
gtccccacaccaaagttgtgcg

SEQ ID NO: 30 - DNA, 24 bp, Artificial Sequence
  primer
tggatgggccccgagaacctcgaa

SEQ ID NO: 31 - DNA, 27 bp, Artificial Sequence
  primer
atcgtactgagaagcactccacaatgc

SEQ ID NO: 32 - DNA, 19 bp, Artificial Sequence
  primer
gggttccctaagggttgga

SEQ ID NO: 33 - DNA, 23 bp, Artificial Sequence
  primer
gtgccagcaagatccaatctaga

SEQ ID NO: 34 - DNA, 19 bp, Artificial Sequence
  primer
tccaacccttagggaaccc

SEQ ID NO: 35 - DNA, 41 bp, Artificial Sequence
  primer
aagcccttcagcggccagtagctccaacccttagggaaccc

SEQ ID NO: 36 - DNA, 42 bp, Artificial Sequence
  primer
gtgaaaagctccgggtcttaggctccaacccttagggaaccc

SEQ ID NO: 37 - DNA, 45 bp, Artificial Sequence
  primer
gtgccagcaagatccaatctagaggcgccttccatggagacgcag

SEQ ID NO: 38 - DNA, 48 bp, Artificial Sequence
  primer
gtgccagcaagatccaatctagaattccgctgaccatcaataaggaag

SEQ ID NO: 39 - DNA, 49 bp, Artificial Sequence
  primer
gtgccagcaagatccaatctagaagccactggatttaagcagagttcaa

SEQ ID NO: 40 - DNA, 46 bp, Artificial Sequence
  primer
gtgccagcaagatccaatctagaactgaaggcagccttcgacgtca

SEQ ID NO: 41 - DNA, 47 bp, Artificial Sequence
  primer
gtgccagcaagatccaatctagagctcttgcatcacccaggggaaag

SEQ ID NO: 42 - DNA, 43 bp, Artificial Sequence
  primer
gtgccagcaagatccaatctagacagtggcgccggggaggcag

SEQ ID NO: 43 - DNA, 44 bp, Artificial Sequence
  primer
ccattgagacccagagcagcagttctccaacccttagggaaccc

SEQ ID NO: 44 - DNA, 47 bp, Artificial Sequence
  primer
gtgccagcaagatccaatctagagagtttgatgaggagcgagcccag

SEQ ID NO: 45 - DNA, 48 bp, Artificial Sequence
  primer
gtgccagcaagatccaatctagacaggtctcatcgggaggaaatggag

SEQ ID NO: 46 - DNA, 49 bp, Artificial Sequence
  primer
gtgccagcaagatccaatctagaagtttcacagctgctggcagtaactg

SEQ ID NO: 47 - DNA, 46 bp, Artificial Sequence
  primer
gccaaggcgaacctagacaagaataagtccaacccttagggaaccc

SEQ ID NO: 48 - DNA, 45 bp, Artificial Sequence
  primer
aatgaagttgagagcgtcacagggattccaacccttagggaaccc

SEQ ID NO: 49 - DNA, 44 bp, Artificial Sequence
  primer
gagctgcttcaagaagaaacccggctccaacccttagggaaccc

SEQ ID NO: 50 - DNA, 44 bp, Artificial Sequence
  primer
ctctccgactcgaagaagaagctgctccaacccttagggaaccc

SEQ ID NO: 51 - DNA, 48 bp, Artificial Sequence
  primer
ttgttagccgaggagaaaaacatctcttctccaacccttagggaaccc

SEQ ID NO: 52 - DNA, 44 bp, Artificial Sequence
  primer
gtccatgagctggagaagtccaagctccaacccttagggaaccc

SEQ ID NO: 53 - DNA, 47 bp, Artificial Sequence
  primer
cttcacgagtatgagacggaactggaagtccaacccttagggaaccc

SEQ ID NO: 54 - DNA, 48 bp, Artificial Sequence
  primer
gtgccagcaagatccaatctagatctcggcctcccgactcctacagtg

SEQ ID NO: 55 - DNA, 45 bp, Artificial Sequence
  primer
ttttgagtatccgaggagcccaggagtccaacccttagggaaccc

SEQ ID NO: 56 - DNA, 52 bp, Artificial Sequence
  primer
gtgccagcaagatccaatctagaggtcatactgcatcagaaccatgaagaag

SEQ ID NO: 57 - DNA, 47 bp, Artificial Sequence
  primer
gtgccagcaagatccaatctagaccatgcccattgggagaatagcag

SEQ ID NO: 58 - DNA, 49 bp, Artificial Sequence
  primer
aatgcatacttggaatgaatccttctagagtccaacccttagggaaccc

SEQ ID NO: 59 - DNA, 42 bp, Artificial Sequence
  primer
atgccagcacgagccgccgcttctccaacccttagggaaccc

SEQ ID NO: 60 - DNA, 46 bp, Artificial Sequence
  primer
gtgccagcaagatccaatctagaacctcagctccgcggaagttgcg

SEQ ID NO: 61 - DNA, 41 bp, Artificial Sequence
  primer
atcgcccaggaccacaccgcagtccaacccttagggaaccc

SEQ ID NO: 62 - DNA, 41 bp, Artificial Sequence
  primer
gatgaccgagcggccgccgagctccaacccttagggaaccc

SEQ ID NO: 63 - DNA, 41 bp, Artificial Sequence
  primer
gtccccacaccaaagttgtgcgtccaacccttagggaaccc

SEQ ID NO: 64 - DNA, 47 bp, Artificial Sequence
  primer
gtgccagcaagatccaatctagatggatgggccccgagaacctcgaa

SEQ ID NO: 65 - DNA, 46 bp, Artificial Sequence
  primer
atcgtactgagaagcactccacaatgctccaacccttagggaaccc

SEQ ID NO: 66 - DNA, 23 bp, Artificial Sequence
  primer
gcagatggccagtcaggcaccag

SEQ ID NO: 67 - DNA, 29 bp, Artificial Sequence
  primer
tcagttatcatctggtgacaaagcttcag

SEQ ID NO: 68 - DNA, 27 bp, Artificial Sequence
  primer
cgtcttctaatttcactgctgcacaag

SEQ ID NO: 69 - DNA, 24 bp, Artificial Sequence
  primer
ggttcagcttttgccaagcttcag

SEQ ID NO: 70 - DNA, 25 bp, Artificial Sequence
  primer
ggctttggatccacagctacctcaa

SEQ ID NO: 71 - DNA, 26 bp, Artificial Sequence
  primer
tgggttttcctctccaaacaaaacag

SEQ ID NO: 72 - DNA, 23 bp, Artificial Sequence
  primer
tggttttggatcaggcacaggag

SEQ ID NO: 73 - DNA, 30 bp, Artificial Sequence
  primer
attatgaactattaacagaaaatgacatgt

SEQ ID NO: 74 - DNA, 28 bp, Artificial Sequence
  primer
ttcttcaggagagaataccatgggtacc

SEQ ID NO: 75 - DNA, 30 bp, Artificial Sequence
  primer
gaaattgaacttagctcattaagggaagct

SEQ ID NO: 76 - DNA, 30 bp, Artificial Sequence
  primer
cgagaaaatgtcattgaatataaacactgt

SEQ ID NO: 77 - DNA, 30 bp, Artificial Sequence
  primer
cctagtgagagccttgctactactgatgat

SEQ ID NO: 78 - DNA, 30 bp, Artificial Sequence
  primer
actgaatctccagtgttagtgaatgactat

SEQ ID NO: 79 - DNA, 30 bp, Artificial Sequence
  primer
cagtgcatattagtggacagcacttagtag

SEQ ID NO: 80 - DNA, 28 bp, Artificial Sequence
  primer
ctgagaatgcacttactggctcattcag

SEQ ID NO: 81 - DNA, 26 bp, Artificial Sequence
  primer
ggagacacacaggcagacccatactg

SEQ ID NO: 82 - DNA, 28 bp, Artificial Sequence
  primer
atcaccattgcttggaagtttgattctc

SEQ ID NO: 83 - DNA, 26 bp, Artificial Sequence
  primer
accagttccctgcgagtctgctactg

SEQ ID NO: 84 - DNA, 27 bp, Artificial Sequence
  primer
attacctggtcatgatcattgtccgtg

SEQ ID NO: 85 - DNA, 26 bp, Artificial Sequence
  primer
tgctacagttgaaactccagcagcgc

SEQ ID NO: 86 - DNA, 26 bp, Artificial Sequence
  primer
ctttgaaaagtccagccgcatttcat

SEQ ID NO: 87 - DNA, 30 bp, Artificial Sequence
  primer
gaagtcacaatgaaacagatttgcaaaaag

SEQ ID NO: 88 - DNA, 25 bp, Artificial Sequence
  primer
gaaattcggcgccttcatcagtatg

SEQ ID NO: 89 - DNA, 30 bp, Artificial Sequence
  primer
aagaagatgaagagtcagatgatgctgatg

SEQ ID NO: 90 - DNA, 30 bp, Artificial Sequence
  primer
attttggatcattgtttgacttggaaaatg

SEQ ID NO: 91 - DNA, 26 bp, Artificial Sequence
  primer
atgggaataactgggaacacaagtcc

SEQ ID NO: 92 - DNA, 23 bp, Artificial Sequence
  primer
gcttggtgcaggatttggaacag

SEQ ID NO: 93 - DNA, 22 bp, Artificial Sequence
  primer
ggagccccccaggccccagtag

SEQ ID NO: 94 - DNA, 28 bp, Artificial Sequence
  primer
gatgtcagaccctaagaagaaggaagag

SEQ ID NO: 95 - DNA, 24 bp, Artificial Sequence
  primer
agccaatggagcattcatgcccaa

SEQ ID NO: 96 - DNA, 23 bp, Artificial Sequence
  primer
ctgtgcggtcagagaagaaacgc

SEQ ID NO: 97 - DNA, 27 bp, Artificial Sequence
  primer
ttgatagagaaaaacaacccagcgaag

SEQ ID NO: 98 - DNA, 24 bp, Artificial Sequence
  primer
ataacccagcagccaactggcttc

SEQ ID NO: 99 - DNA, 27 bp, Artificial Sequence
  primer
gatggtaaattgaaaaaacccaagaat

SEQ ID NO: 100 - DNA, 27 bp, Artificial Sequence
  primer
ttcttgatgaagcagatagaatcttgg

SEQ ID NO: 101 - DNA, 23 bp, Artificial Sequence
  primer
cacccctgccactttggaacaga

SEQ ID NO: 102 - DNA, 29 bp, Artificial Sequence
  primer
ataatctcagtgataccttgaagaagctg

SEQ ID NO: 103 - DNA, 28 bp, Artificial Sequence
  primer
atacggaaacaagtgaaaaaatccaagc

SEQ ID NO: 104 - DNA, 29 bp, Artificial Sequence
  primer
accaagaggctattcaagatctctgtctg

SEQ ID NO: 105 - DNA, 29 bp, Artificial Sequence
  primer
accaagaggctattcaagatctctgcatg

SEQ ID NO: 106 - DNA, 30 bp, Artificial Sequence
  primer
gcagtggaggaagtctctttaagaaaatag

SEQ ID NO: 107 - DNA, 23 bp, Artificial Sequence
  primer
cttctgccgctgcttctgcacag

SEQ ID NO: 108 - DNA, 23 bp, Artificial Sequence
  primer
gcatggggcggctggttctgctg

SEQ ID NO: 109 - DNA, 24 bp, Artificial Sequence
  primer
ctgaggacatctggaggaaggctg

SEQ ID NO: 110 - DNA, 21 bp, Artificial Sequence
  primer
cagcaggaggactccagcgag

SEQ ID NO: 111 - DNA, 24 bp, Artificial Sequence
  primer
gcttcctgctgaactccaagttcc

SEQ ID NO: 112 - DNA, 23 bp, Artificial Sequence
  primer
gacttccagccactgcgctattt

SEQ ID NO: 113 - DNA, 30 bp, Artificial Sequence
  primer
atccctgtaaaacaaaaaccaaaagaaaag

SEQ ID NO: 114 - DNA, 28 bp, Artificial Sequence
  primer
agtccacaggatcagagtggactttaag

SEQ ID NO: 115 - DNA, 27 bp, Artificial Sequence
  primer
ctctgtgccagtagtgggcatgtagag

SEQ ID NO: 116 - DNA, 26 bp, Artificial Sequence
  primer
gtggaaggcaacatcaggctacaaag

SEQ ID NO: 117 - DNA, 26 bp, Artificial Sequence
  primer
cagacctactccaatgaagtccattg

SEQ ID NO: 118 - DNA, 23 bp, Artificial Sequence
  primer
gaaatgacccattcatggccgcc

SEQ ID NO: 119 - DNA, 26 bp, Artificial Sequence
  primer
gactctcagcatgtcagttctgtaac

SEQ ID NO: 120 - DNA, 26 bp, Artificial Sequence
  primer
cctgagcctccaacaacaaacaaatg

SEQ ID NO: 121 - DNA, 27 bp, Artificial Sequence
  primer
gatgagcaattcttaggttttggctca

SEQ ID NO: 122 - DNA, 25 bp, Artificial Sequence
  primer
gaagaacctaggaaagtccgctttg

SEQ ID NO: 123 - DNA, 27 bp, Artificial Sequence
  primer
gaccctaataggagtattcataccagc

SEQ ID NO: 124 - DNA, 23 bp, Artificial Sequence
  primer
tctgaacaacccagtcctgccag

SEQ ID NO: 125 - DNA, 31 bp, Artificial Sequence
  primer
attcttgaagtgaaaagtccaataaagcaaa

SEQ ID NO: 126 - DNA, 25 bp, Artificial Sequence
  primer
gcatacctagatgaactggtagagc

SEQ ID NO: 127 - DNA, 23 bp, Artificial Sequence
  primer
tgcaccgtccaggtgaggttaga

SEQ ID NO: 128 - DNA, 23 bp, Artificial Sequence
  primer
gaggagccgaggaaggtctgctt

SEQ ID NO: 129 - DNA, 23 bp, Artificial Sequence
  primer
gtgatggtaatgcccgaaggagc

SEQ ID NO: 130 - DNA, 23 bp, Artificial Sequence
  primer
gacgccaacaaggagagcagcaa

SEQ ID NO: 131 - DNA, 23 bp, Artificial Sequence
  primer
tctgcccagtcaagcccgtccaa

SEQ ID NO: 132 - DNA, 27 bp, Artificial Sequence
  primer
agatgtgaactttgtccccataaggat

SEQ ID NO: 133 - DNA, 29 bp, Artificial Sequence
  primer
acttgctacatttgtgatgaacaaggaag

SEQ ID NO: 134 - DNA, 31 bp, Artificial Sequence
  primer
aaatataaagagaaggacaaacacaaacaga

SEQ ID NO: 135 - DNA, 30 bp, Artificial Sequence
  primer
acttatacaagcactagcaacaactctata

SEQ ID NO: 136 - DNA, 27 bp, Artificial Sequence
  primer
gcaaatactctatctggatcttctctc

SEQ ID NO: 137 - DNA, 26 bp, Artificial Sequence
  primer
gatttggagttccatggagtgatgag

SEQ ID NO: 138 - DNA, 27 bp, Artificial Sequence
  primer
gattctgtttcactgaggccatctatc

SEQ ID NO: 139 - DNA, 23 bp, Artificial Sequence
  primer
cacatctccatcccccagcctga

SEQ ID NO: 140 - DNA, 24 bp, Artificial Sequence
  primer
gtggccaacatgagtgctaaggac

SEQ ID NO: 141 - DNA, 31 bp, Artificial Sequence
  primer
ttatcaagtgggaatcctgtatatgaaaaat

SEQ ID NO: 142 - DNA, 27 bp, Artificial Sequence
  primer
catgataccagtagtcctttgctaatc

SEQ ID NO: 143 - DNA, 27 bp, Artificial Sequence
  primer
agtcgaaaggacaaagaacgccttaag

SEQ ID NO: 144 - DNA, 23 bp, Artificial Sequence
  primer
gtctcctcctcggcttcctcttc

SEQ ID NO: 145 - DNA, 24 bp, Artificial Sequence
  primer
gtgttttctctggctggctctacc

SEQ ID NO: 146 - DNA, 23 bp, Artificial Sequence
  primer
ggtgaaggttgccgaactgtccc

SEQ ID NO: 147 - DNA, 23 bp, Artificial Sequence
  primer
gaagctatgagggaccctgtgag

SEQ ID NO: 148 - DNA, 24 bp, Artificial Sequence
  primer
gcacggacacttgctagtatgttg

SEQ ID NO: 149 - DNA, 24 bp, Artificial Sequence
  primer
gcatcagcttctggtgatgtgagc

SEQ ID NO: 150 - DNA, 23 bp, Artificial Sequence
  primer
gagatgacgcattcatggcctcc

SEQ ID NO: 151 - DNA, 24 bp, Artificial Sequence
  primer
gagtctcagcagtccaattttggc

SEQ ID NO: 152 - DNA, 26 bp, Artificial Sequence
  primer
atatttaacaccgtgcccgatatgcc

SEQ ID NO: 153 - DNA, 27 bp, Artificial Sequence
  primer
aagatggcagtgaacgtatactcaacg

SEQ ID NO: 154 - DNA, 30 bp, Artificial Sequence
  primer
ataattcctgtggacaaattagtaaaagga

SEQ ID NO: 155 - DNA, 30 bp, Artificial Sequence
  primer
gtcaacgtattgaaacttactgttgaagac

SEQ ID NO: 156 - DNA, 24 bp, Artificial Sequence
  primer
gacattgacaagcagtacgtgggc

SEQ ID NO: 157 - DNA, 23 bp, Artificial Sequence
  primer
gaggcacgcggacctccagtggc

SEQ ID NO: 158 - DNA, 27 bp, Artificial Sequence
  primer
ccttgaaaagatcttttgaggtcgagg

SEQ ID NO: 159 - DNA, 31 bp, Artificial Sequence
  primer
gaaaaaaaccttgaagataacttacagagtt

SEQ ID NO: 160 - DNA, 26 bp, Artificial Sequence
  primer
gagagtagatctggagaaaccaacag

SEQ ID NO: 161 - DNA, 23 bp, Artificial Sequence
  primer
gagatgacctggcttccaccact

SEQ ID NO: 162 - DNA, 23 bp, Artificial Sequence
  primer
caggcagctcagagaacggctct

SEQ ID NO: 163 - DNA, 31 bp, Artificial Sequence
  primer
atttttgatcaccatactgaagaggatatag

SEQ ID NO: 164 - DNA, 23 bp, Artificial Sequence
  primer
aactccatccggcacaacctgtc

SEQ ID NO: 165 - DNA, 27 bp, Artificial Sequence
  primer
ctccagggttccttgaaaagaaaacag

SEQ ID NO: 166 - DNA, 24 bp, Artificial Sequence
  primer
gatcaacactctgtggtaggccag

SEQ ID NO: 167 - DNA, 28 bp, Artificial Sequence
  primer
caagttggaattgacagaggtgatatac

SEQ ID NO: 168 - DNA, 23 bp, Artificial Sequence
  primer
gcccctagcagtcttcttgatgc

SEQ ID NO: 169 - DNA, 26 bp, Artificial Sequence
  primer
caacctcataatattctgcagaggcg

SEQ ID NO: 170 - DNA, 23 bp, Artificial Sequence
  primer
gacatgcggaagcacgtggccat

SEQ ID NO: 171 - DNA, 23 bp, Artificial Sequence
  primer
ggctacatgcagccgctgaagca

SEQ ID NO: 172 - DNA, 27 bp, Artificial Sequence
  primer
ttctccaaggatgtcctagtaaacatc

SEQ ID NO: 173 - DNA, 24 bp, Artificial Sequence
  primer
caaagcatgcgtgagaacaaggag

SEQ ID NO: 174 - DNA, 25 bp, Artificial Sequence
  primer
ccagaagtcattggatctgtgtcac

SEQ ID NO: 175 - DNA, 30 bp, Artificial Sequence
  primer
gtaaccatggagcttattacagataacaaa

SEQ ID NO: 176 - DNA, 24 bp, Artificial Sequence
  primer
gtgattcctgtctctctgtcttcc

SEQ ID NO: 177 - DNA, 23 bp, Artificial Sequence
  primer
gcagaggaccgaggaaatggact

SEQ ID NO: 178 - DNA, 24 bp, Artificial Sequence
  primer
gatggagctgtagttacaccctcc

SEQ ID NO: 179 - DNA, 24 bp, Artificial Sequence
  primer
agtcccaagagtggcccaaaagag

SEQ ID NO: 180 - DNA, 25 bp, Artificial Sequence
  primer
gacactcaatcacttgtcggaagtc

SEQ ID NO: 181 - DNA, 23 bp, Artificial Sequence
  primer
gtggaacggccgccttctccatt

SEQ ID NO: 182 - DNA, 23 bp, Artificial Sequence
  primer
gtgaatgaggcctctggggatgg

SEQ ID NO: 183 - DNA, 23 bp, Artificial Sequence
  primer
aagcctggaatggtcccccctcc

SEQ ID NO: 184 - DNA, 23 bp, Artificial Sequence
  primer
aactcgatccgccacaacctgtc

SEQ ID NO: 185 - DNA, 28 bp, Artificial Sequence
  primer
caacagccaactcagtttataaatccag

SEQ ID NO: 186 - DNA, 25 bp, Artificial Sequence
  primer
ctgcagaagaaagatcagcaactgg

SEQ ID NO: 187 - DNA, 26 bp, Artificial Sequence
  primer
atgggaataactgggaacacaagtcc

SEQ ID NO: 188 - DNA, 27 bp, Artificial Sequence
  primer
tctcagatgcaaacatcagtgggaatt

SEQ ID NO: 189 - DNA, 25 bp, Artificial Sequence
  primer
atgatggaggaggatttgcaaggag

SEQ ID NO: 190 - DNA, 25 bp, Artificial Sequence
  primer
gtgagtacccagaacatgaagatgg

SEQ ID NO: 191 - DNA, 23 bp, Artificial Sequence
  primer
ggcctggagcaggatgtcctcca

SEQ ID NO: 192 - DNA, 27 bp, Artificial Sequence
  primer
aatcagatgggtgactcaaatatctcc

SEQ ID NO: 193 - DNA, 24 bp, Artificial Sequence
  primer
tctgctccatctggacacaagcat

SEQ ID NO: 194 - DNA, 23 bp, Artificial Sequence
  primer
ctggagaatgctggaggagacct

SEQ ID NO: 195 - DNA, 26 bp, Artificial Sequence
  primer
aatactctggagcagtgcaatgtgtg

SEQ ID NO: 196 - DNA, 26 bp, Artificial Sequence
  primer
aatactctggagcagtgcaatgtgtg

SEQ ID NO: 197 - DNA, 27 bp, Artificial Sequence
  primer
agagaaatagcccggaaacttgcaaat

SEQ ID NO: 198 - DNA, 29 bp, Artificial Sequence
  primer
aatgaagagcttcgaaacttgtctttgtc

SEQ ID NO: 199 - DNA, 24 bp, Artificial Sequence
  primer
ccacagcgtcctgtgtttactcat

SEQ ID NO: 200 - DNA, 30 bp, Artificial Sequence
  primer
ttccaaaagctgagacaagatcttgaaatg

SEQ ID NO: 201 - DNA, 24 bp, Artificial Sequence
  primer
gtgacgacgtcatcaggaagcaag

SEQ ID NO: 202 - DNA, 23 bp, Artificial Sequence
  primer
gacaacagccggcgtgtggagca

SEQ ID NO: 203 - DNA, 24 bp, Artificial Sequence
  primer
gacttcctgacagacctgatgatg

SEQ ID NO: 204 - DNA, 30 bp, Artificial Sequence
  primer
gatcagtttgacaacttagaaaaacacaca

SEQ ID NO: 205 - DNA, 27 bp, Artificial Sequence
  primer
gctacagacaagagaaaagctttagag

SEQ ID NO: 206 - DNA, 28 bp, Artificial Sequence
  primer
actgtggatattcataaggagaaagtgg

SEQ ID NO: 207 - DNA, 30 bp, Artificial Sequence
  primer
gtcaagtacaaaagagattttgaagaaagc

SEQ ID NO: 208 - DNA, 31 bp, Artificial Sequence
  primer
gtaaaataccatgaagattttgaaaaaacaa

SEQ ID NO: 209 - DNA, 23 bp, Artificial Sequence
  primer
aagcggtaccgcgcggtgtatga

SEQ ID NO: 210 - DNA, 23 bp, Artificial Sequence
  primer
ctggtcagtgagaaggtcggagg

SEQ ID NO: 211 - DNA, 27 bp, Artificial Sequence
  primer
ttctccaatatccccttcttcatcttc

SEQ ID NO: 212 - DNA, 23 bp, Artificial Sequence
  primer
ggccctcctcaggacctgtctgt

SEQ ID NO: 213 - DNA, 23 bp, Artificial Sequence
  primer
ggcagcaaggagcgcttccactg

SEQ ID NO: 214 - DNA, 24 bp, Artificial Sequence
  primer
acctacatcggctctgtgctcatc

SEQ ID NO: 215 - DNA, 23 bp, Artificial Sequence
  primer
accttcacggcatggtgcaactc

SEQ ID NO: 216 - DNA, 23 bp, Artificial Sequence
  primer
cctatgggctatgggcctcgtat

SEQ ID NO: 217 - DNA, 25 bp, Artificial Sequence
  primer
gcacaaatgtctagttcttcctgcc

SEQ ID NO: 218 - DNA, 24 bp, Artificial Sequence
  primer
ctttcccagccagctgtaagcatt

SEQ ID NO: 219 - DNA, 28 bp, Artificial Sequence
  primer
gaaacaatgaccgataaaacagagaagg

SEQ ID NO: 220 - DNA, 46 bp, Artificial Sequence
  primer
gtgccagcaagatccaatctagagcagatggccagtcaggcaccag

SEQ ID NO: 221 - DNA, 52 bp, Artificial Sequence
  primer
gtgccagcaagatccaatctagatcagttatcatctggtgacaaagcttcag

SEQ ID NO: 222 - DNA, 50 bp, Artificial Sequence
  primer
gtgccagcaagatccaatctagacgtcttctaatttcactgctgcacaag

SEQ ID NO: 223 - DNA, 47 bp, Artificial Sequence
  primer
gtgccagcaagatccaatctagaggttcagcttttgccaagcttcag

SEQ ID NO: 224 - DNA, 48 bp, Artificial Sequence
  primer
gtgccagcaagatccaatctagaggctttggatccacagctacctcaa

SEQ ID NO: 225 - DNA, 49 bp, Artificial Sequence
  primer
gtgccagcaagatccaatctagatgggttttcctctccaaacaaaacag

SEQ ID NO: 226 - DNA, 46 bp, Artificial Sequence
  primer
gtgccagcaagatccaatctagatggttttggatcaggcacaggag

SEQ ID NO: 227 - DNA, 49 bp, Artificial Sequence
  primer
attatgaactattaacagaaaatgacatgttccaacccttagggaaccc

SEQ ID NO: 228 - DNA, 47 bp, Artificial Sequence
  primer
ttcttcaggagagaataccatgggtacctccaacccttagggaaccc

SEQ ID NO: 229 - DNA, 49 bp, Artificial Sequence
  primer
gaaattgaacttagctcattaagggaagcttccaacccttagggaaccc

SEQ ID NO: 230 - DNA, 49 bp, Artificial Sequence
  primer
cgagaaaatgtcattgaatataaacactgttccaacccttagggaaccc

SEQ ID NO: 231 - DNA, 53 bp, Artificial Sequence
  primer
gtgccagcaagatccaatctagacctagtgagagccttgctactactgatgat

SEQ ID NO: 232 - DNA, 53 bp, Artificial Sequence
  primer
gtgccagcaagatccaatctagaactgaatctccagtgttagtgaatgactat

SEQ ID NO: 233 - DNA, 53 bp, Artificial Sequence
  primer
gtgccagcaagatccaatctagacagtgcatattagtggacagcacttagtag

SEQ ID NO: 234 - DNA, 51 bp, Artificial Sequence
  primer
gtgccagcaagatccaatctagactgagaatgcacttactggctcattcag

SEQ ID NO: 235 - DNA, 49 bp, Artificial Sequence
  primer
gtgccagcaagatccaatctagaggagacacacaggcagacccatactg

SEQ ID NO: 236 - DNA, 51 bp, Artificial Sequence
  primer
gtgccagcaagatccaatctagaatcaccattgcttggaagtttgattctc

SEQ ID NO: 237 - DNA, 49 bp, Artificial Sequence
  primer
gtgccagcaagatccaatctagaaccagttccctgcgagtctgctactg

SEQ ID NO: 238 - DNA, 50 bp, Artificial Sequence
  primer
gtgccagcaagatccaatctagaattacctggtcatgatcattgtccgtg

SEQ ID NO: 239 - DNA, 45 bp, Artificial Sequence
  primer
tgctacagttgaaactccagcagcgctccaacccttagggaaccc

SEQ ID NO: 240 - DNA, 45 bp, Artificial Sequence
  primer
ctttgaaaagtccagccgcatttcattccaacccttagggaaccc

SEQ ID NO: 241 - DNA, 53 bp, Artificial Sequence
  primer
gtgccagcaagatccaatctagagaagtcacaatgaaacagatttgcaaaaag

SEQ ID NO: 242 - DNA, 44 bp, Artificial Sequence
  primer
gaaattcggcgccttcatcagtatgtccaacccttagggaaccc

SEQ ID NO: 243 - DNA, 53 bp, Artificial Sequence
  primer
gtgccagcaagatccaatctagaaagaagatgaagagtcagatgatgctgatg

SEQ ID NO: 244 - DNA, 49 bp, Artificial Sequence
  primer
attttggatcattgtttgacttggaaaatgtccaacccttagggaaccc

SEQ ID NO: 245 - DNA, 45 bp, Artificial Sequence
  primer
atgggaataactgggaacacaagtcctccaacccttagggaaccc

SEQ ID NO: 246 - DNA, 46 bp, Artificial Sequence
  primer
gtgccagcaagatccaatctagagcttggtgcaggatttggaacag

SEQ ID NO: 247 - DNA, 45 bp, Artificial Sequence
  primer
gtgccagcaagatccaatctagaggagccccccaggccccagtag

SEQ ID NO: 248 - DNA, 51 bp, Artificial Sequence
  primer
gtgccagcaagatccaatctagagatgtcagaccctaagaagaaggaagag

SEQ ID NO: 249 - DNA, 47 bp, Artificial Sequence
  primer
gtgccagcaagatccaatctagaagccaatggagcattcatgcccaa

SEQ ID NO: 250 - DNA, 42 bp, Artificial Sequence
  primer
ctgtgcggtcagagaagaaacgctccaacccttagggaaccc

SEQ ID NO: 251 - DNA, 46 bp, Artificial Sequence
  primer
ttgatagagaaaaacaacccagcgaagtccaacccttagggaaccc

SEQ ID NO: 252 - DNA, 43 bp, Artificial Sequence
  primer
ataacccagcagccaactggcttctccaacccttagggaaccc

SEQ ID NO: 253 - DNA, 46 bp, Artificial Sequence
  primer
gatggtaaattgaaaaaacccaagaattccaacccttagggaaccc

SEQ ID NO: 254 - DNA, 46 bp, Artificial Sequence
  primer
ttcttgatgaagcagatagaatcttggtccaacccttagggaaccc

SEQ ID NO: 255 - DNA, 42 bp, Artificial Sequence
  primer
cacccctgccactttggaacagatccaacccttagggaaccc

SEQ ID NO: 256 - DNA, 48 bp, Artificial Sequence
  primer
ataatctcagtgataccttgaagaagctgtccaacccttagggaaccc

SEQ ID NO: 257 - DNA, 47 bp, Artificial Sequence
  primer
atacggaaacaagtgaaaaaatccaagctccaacccttagggaaccc

SEQ ID NO: 258 - DNA, 52 bp, Artificial Sequence
  primer
gtgccagcaagatccaatctagaaccaagaggctattcaagatctctgtctg

SEQ ID NO: 259 - DNA, 52 bp, Artificial Sequence
  primer
gtgccagcaagatccaatctagaaccaagaggctattcaagatctctgcatg

SEQ ID NO: 260 - DNA, 49 bp, Artificial Sequence
  primer
gcagtggaggaagtctctttaagaaaatagtccaacccttagggaaccc

SEQ ID NO: 261 - DNA, 46 bp, Artificial Sequence
  primer
gtgccagcaagatccaatctagacttctgccgctgcttctgcacag

SEQ ID NO: 262 - DNA, 42 bp, Artificial Sequence
  primer
gcatggggcggctggttctgctgtccaacccttagggaaccc

SEQ ID NO: 263 - DNA, 47 bp, Artificial Sequence
  primer
gtgccagcaagatccaatctagactgaggacatctggaggaaggctg

SEQ ID NO: 264 - DNA, 44 bp, Artificial Sequence
  primer
gtgccagcaagatccaatctagacagcaggaggactccagcgag

SEQ ID NO: 265 - DNA, 43 bp, Artificial Sequence
  primer
gcttcctgctgaactccaagttcctccaacccttagggaaccc

SEQ ID NO: 266 - DNA, 42 bp, Artificial Sequence
  primer
gacttccagccactgcgctattttccaacccttagggaaccc

SEQ ID NO: 267 - DNA, 53 bp, Artificial Sequence
  primer
gtgccagcaagatccaatctagaatccctgtaaaacaaaaaccaaaagaaaag

SEQ ID NO: 268 - DNA, 51 bp, Artificial Sequence
  primer
gtgccagcaagatccaatctagaagtccacaggatcagagtggactttaag

SEQ ID NO: 269 - DNA, 50 bp, Artificial Sequence
  primer
gtgccagcaagatccaatctagactctgtgccagtagtgggcatgtagag

SEQ ID NO: 270 - DNA, 49 bp, Artificial Sequence
  primer
gtgccagcaagatccaatctagagtggaaggcaacatcaggctacaaag

SEQ ID NO: 271 - DNA, 45 bp, Artificial Sequence
  primer
cagacctactccaatgaagtccattgtccaacccttagggaaccc

SEQ ID NO: 272 - DNA, 42 bp, Artificial Sequence
  primer
gaaatgacccattcatggccgcctccaacccttagggaaccc

SEQ ID NO: 273 - DNA, 45 bp, Artificial Sequence
  primer
gactctcagcatgtcagttctgtaactccaacccttagggaaccc

SEQ ID NO: 274 - DNA, 45 bp, Artificial Sequence
  primer
cctgagcctccaacaacaaacaaatgtccaacccttagggaaccc

SEQ ID NO: 275 - DNA, 46 bp, Artificial Sequence
  primer
gatgagcaattcttaggttttggctcatccaacccttagggaaccc

SEQ ID NO: 276 - DNA, 44 bp, Artificial Sequence
  primer
gaagaacctaggaaagtccgctttgtccaacccttagggaaccc

SEQ ID NO: 277 - DNA, 46 bp, Artificial Sequence
  primer
gaccctaataggagtattcataccagctccaacccttagggaaccc

SEQ ID NO: 278 - DNA, 42 bp, Artificial Sequence
  primer
tctgaacaacccagtcctgccagtccaacccttagggaaccc

SEQ ID NO: 279 - DNA, 50 bp, Artificial Sequence
  primer
attcttgaagtgaaaagtccaataaagcaaatccaacccttagggaaccc

SEQ ID NO: 280 - DNA, 44 bp, Artificial Sequence
  primer
gcatacctagatgaactggtagagctccaacccttagggaaccc

SEQ ID NO: 281 - DNA, 42 bp, Artificial Sequence
  primer
tgcaccgtccaggtgaggttagatccaacccttagggaaccc

SEQ ID NO: 282 - DNA, 42 bp, Artificial Sequence
  primer
gaggagccgaggaaggtctgctttccaacccttagggaaccc

SEQ ID NO: 283 - DNA, 42 bp, Artificial Sequence
  primer
gtgatggtaatgcccgaaggagctccaacccttagggaaccc

SEQ ID NO: 284 - DNA, 42 bp, Artificial Sequence
  primer
gacgccaacaaggagagcagcaatccaacccttagggaaccc

SEQ ID NO: 285 - DNA, 42 bp, Artificial Sequence
  primer
tctgcccagtcaagcccgtccaatccaacccttagggaaccc

SEQ ID NO: 286 - DNA, 46 bp, Artificial Sequence
  primer
agatgtgaactttgtccccataaggattccaacccttagggaaccc

SEQ ID NO: 287 - DNA, 48 bp, Artificial Sequence
  primer
acttgctacatttgtgatgaacaaggaagtccaacccttagggaaccc

SEQ ID NO: 288 - DNA, 50 bp, Artificial Sequence
  primer
aaatataaagagaaggacaaacacaaacagatccaacccttagggaaccc

SEQ ID NO: 289 - DNA, 49 bp, Artificial Sequence
  primer
acttatacaagcactagcaacaactctatatccaacccttagggaaccc

SEQ ID NO: 290 - DNA, 46 bp, Artificial Sequence
  primer
gcaaatactctatctggatcttctctctccaacccttagggaaccc

SEQ ID NO: 291 - DNA, 45 bp, Artificial Sequence
  primer
gatttggagttccatggagtgatgagtccaacccttagggaaccc

SEQ ID NO: 292 - DNA, 46 bp, Artificial Sequence
  primer
gattctgtttcactgaggccatctatctccaacccttagggaaccc

SEQ ID NO: 293 - DNA, 42 bp, Artificial Sequence
  primer
cacatctccatcccccagcctgatccaacccttagggaaccc

SEQ ID NO: 294 - DNA, 43 bp, Artificial Sequence
  primer
gtggccaacatgagtgctaaggactccaacccttagggaaccc

SEQ ID NO: 295 - DNA, 50 bp, Artificial Sequence
  primer
ttatcaagtgggaatcctgtatatgaaaaattccaacccttagggaaccc

SEQ ID NO: 296 - DNA, 46 bp, Artificial Sequence
  primer
catgataccagtagtcctttgctaatctccaacccttagggaaccc

SEQ ID NO: 297 - DNA, 46 bp, Artificial Sequence
  primer
agtcgaaaggacaaagaacgccttaagtccaacccttagggaaccc

SEQ ID NO: 298 - DNA, 42 bp, Artificial Sequence
  primer
gtctcctcctcggcttcctcttctccaacccttagggaaccc

SEQ ID NO: 299 - DNA, 43 bp, Artificial Sequence
  primer
gtgttttctctggctggctctacctccaacccttagggaaccc

SEQ ID NO: 300 - DNA, 42 bp, Artificial Sequence
  primer
ggtgaaggttgccgaactgtccctccaacccttagggaaccc

SEQ ID NO: 301 - DNA, 42 bp, Artificial Sequence
  primer
gaagctatgagggaccctgtgagtccaacccttagggaaccc

SEQ ID NO: 302 - DNA, 43 bp, Artificial Sequence
  primer
gcacggacacttgctagtatgttgtccaacccttagggaaccc

SEQ ID NO: 303 - DNA, 43 bp, Artificial Sequence
  primer
gcatcagcttctggtgatgtgagctccaacccttagggaaccc

SEQ ID NO: 304 - DNA, 42 bp, Artificial Sequence
  primer
gagatgacgcattcatggcctcctccaacccttagggaaccc

SEQ ID NO: 305 - DNA, 43 bp, Artificial Sequence
  primer
gagtctcagcagtccaattttggctccaacccttagggaaccc

SEQ ID NO: 306 - DNA, 45 bp, Artificial Sequence
  primer
atatttaacaccgtgcccgatatgcctccaacccttagggaaccc

SEQ ID NO: 307 - DNA, 46 bp, Artificial Sequence
  primer
aagatggcagtgaacgtatactcaacgtccaacccttagggaaccc

SEQ ID NO: 308 - DNA, 49 bp, Artificial Sequence
  primer
ataattcctgtggacaaattagtaaaaggatccaacccttagggaaccc

SEQ ID NO: 309 - DNA, 49 bp, Artificial Sequence
  primer
gtcaacgtattgaaacttactgttgaagactccaacccttagggaaccc

SEQ ID NO: 310 - DNA, 43 bp, Artificial Sequence
  primer
gacattgacaagcagtacgtgggctccaacccttagggaaccc

SEQ ID NO: 311 - DNA, 42 bp, Artificial Sequence
  primer
gaggcacgcggacctccagtggctccaacccttagggaaccc

SEQ ID NO: 312 - DNA, 46 bp, Artificial Sequence
  primer
ccttgaaaagatcttttgaggtcgaggtccaacccttagggaaccc

SEQ ID NO: 313 - DNA, 50 bp, Artificial Sequence
  primer
gaaaaaaaccttgaagataacttacagagtttccaacccttagggaaccc

SEQ ID NO: 314 - DNA, 45 bp, Artificial Sequence
  primer
gagagtagatctggagaaaccaacagtccaacccttagggaaccc

SEQ ID NO: 315 - DNA, 42 bp, Artificial Sequence
  primer
gagatgacctggcttccaccacttccaacccttagggaaccc

SEQ ID NO: 316 - DNA, 42 bp, Artificial Sequence
  primer
caggcagctcagagaacggctcttccaacccttagggaaccc

SEQ ID NO: 317 - DNA, 50 bp, Artificial Sequence
  primer
atttttgatcaccatactgaagaggatatagtccaacccttagggaaccc

SEQ ID NO: 318 - DNA, 42 bp, Artificial Sequence
  primer
aactccatccggcacaacctgtctccaacccttagggaaccc

SEQ ID NO: 319 - DNA, 46 bp, Artificial Sequence
  primer
ctccagggttccttgaaaagaaaacagtccaacccttagggaaccc

SEQ ID NO: 320 - DNA, 43 bp, Artificial Sequence
  primer
gatcaacactctgtggtaggccagtccaacccttagggaaccc

SEQ ID NO: 321 - DNA, 47 bp, Artificial Sequence
  primer
caagttggaattgacagaggtgatatactccaacccttagggaaccc

SEQ ID NO: 322 - DNA, 42 bp, Artificial Sequence
  primer
gcccctagcagtcttcttgatgctccaacccttagggaaccc

SEQ ID NO: 323 - DNA, 45 bp, Artificial Sequence
  primer
caacctcataatattctgcagaggcgtccaacccttagggaaccc

SEQ ID NO: 324 - DNA, 42 bp, Artificial Sequence
  primer
gacatgcggaagcacgtggccattccaacccttagggaaccc

SEQ ID NO: 325 - DNA, 42 bp, Artificial Sequence
  primer
ggctacatgcagccgctgaagcatccaacccttagggaaccc

SEQ ID NO: 326 - DNA, 46 bp, Artificial Sequence
  primer
ttctccaaggatgtcctagtaaacatctccaacccttagggaaccc

SEQ ID NO: 327 - DNA, 43 bp, Artificial Sequence
  primer
caaagcatgcgtgagaacaaggagtccaacccttagggaaccc

SEQ ID NO: 328 - DNA, 44 bp, Artificial Sequence
  primer
ccagaagtcattggatctgtgtcactccaacccttagggaaccc

SEQ ID NO: 329 - DNA, 49 bp, Artificial Sequence
  primer
gtaaccatggagcttattacagataacaaatccaacccttagggaaccc

SEQ ID NO: 330 - DNA, 43 bp, Artificial Sequence
  primer
gtgattcctgtctctctgtcttcctccaacccttagggaaccc

SEQ ID NO: 331 - DNA, 42 bp, Artificial Sequence
  primer
gcagaggaccgaggaaatggacttccaacccttagggaaccc

SEQ ID NO: 332 - DNA, 43 bp, Artificial Sequence
  primer
gatggagctgtagttacaccctcctccaacccttagggaaccc

SEQ ID NO: 333 - DNA, 43 bp, Artificial Sequence
  primer
agtcccaagagtggcccaaaagagtccaacccttagggaaccc

SEQ ID NO: 334 - DNA, 44 bp, Artificial Sequence
  primer
gacactcaatcacttgtcggaagtctccaacccttagggaaccc

SEQ ID NO: 335 - DNA, 42 bp, Artificial Sequence
  primer
gtggaacggccgccttctccatttccaacccttagggaaccc

SEQ ID NO: 336 - DNA, 42 bp, Artificial Sequence
  primer
gtgaatgaggcctctggggatggtccaacccttagggaaccc

SEQ ID NO: 337 - DNA, 42 bp, Artificial Sequence
  primer
aagcctggaatggtcccccctcctccaacccttagggaaccc

SEQ ID NO: 338 - DNA, 42 bp, Artificial Sequence
  primer
aactcgatccgccacaacctgtctccaacccttagggaaccc

SEQ ID NO: 339 - DNA, 47 bp, Artificial Sequence
  primer
caacagccaactcagtttataaatccagtccaacccttagggaaccc

SEQ ID NO: 340 - DNA, 44 bp, Artificial Sequence
  primer
ctgcagaagaaagatcagcaactggtccaacccttagggaaccc

SEQ ID NO: 341 - DNA, 45 bp, Artificial Sequence; identical to SEQ ID NO: 245
  primer

SEQ ID NO: 342 - DNA, 46 bp, Artificial Sequence
  primer
tctcagatgcaaacatcagtgggaatttccaacccttagggaaccc

SEQ ID NO: 343 - DNA, 44 bp, Artificial Sequence
  primer
atgatggaggaggatttgcaaggagtccaacccttagggaaccc

SEQ ID NO: 344 - DNA, 44 bp, Artificial Sequence
  primer
gtgagtacccagaacatgaagatggtccaacccttagggaaccc

SEQ ID NO: 345 - DNA, 42 bp, Artificial Sequence
  primer
ggcctggagcaggatgtcctccatccaacccttagggaaccc

SEQ ID NO: 346 - DNA, 46 bp, Artificial Sequence
  primer
aatcagatgggtgactcaaatatctcctccaacccttagggaaccc

SEQ ID NO: 347 - DNA, 43 bp, Artificial Sequence
  primer
tctgctccatctggacacaagcattccaacccttagggaaccc

SEQ ID NO: 348 - DNA, 42 bp, Artificial Sequence
  primer
ctggagaatgctggaggagaccttccaacccttagggaaccc

SEQ ID NO: 349 - DNA, 45 bp, Artificial Sequence
  primer
aatactctggagcagtgcaatgtgtgtccaacccttagggaaccc

SEQ ID NO: 350 - DNA, 45 bp, Artificial Sequence; identical to SEQ ID NO: 349
  primer

SEQ ID NO: 351 - DNA, 46 bp, Artificial Sequence
  primer
agagaaatagcccggaaacttgcaaattccaacccttagggaaccc

SEQ ID NO: 352 - DNA, 48 bp, Artificial Sequence
  primer
aatgaagagcttcgaaacttgtctttgtctccaacccttagggaaccc

SEQ ID NO: 353 - DNA, 43 bp, Artificial Sequence
  primer
ccacagcgtcctgtgtttactcattccaacccttagggaaccc

SEQ ID NO: 354 - DNA, 49 bp, Artificial Sequence
  primer
ttccaaaagctgagacaagatcttgaaatgtccaacccttagggaaccc

SEQ ID NO: 355 - DNA, 43 bp, Artificial Sequence
  primer
gtgacgacgtcatcaggaagcaagtccaacccttagggaaccc

SEQ ID NO: 356 - DNA, 42 bp, Artificial Sequence
  primer
gacaacagccggcgtgtggagcatccaacccttagggaaccc

SEQ ID NO: 357 - DNA, 43 bp, Artificial Sequence
  primer
gacttcctgacagacctgatgatgtccaacccttagggaaccc

SEQ ID NO: 358 - DNA, 49 bp, Artificial Sequence
  primer
gatcagtttgacaacttagaaaaacacacatccaacccttagggaaccc

SEQ ID NO: 359 - DNA, 46 bp, Artificial Sequence
  primer
gctacagacaagagaaaagctttagagtccaacccttagggaaccc

SEQ ID NO: 360 - DNA, 47 bp, Artificial Sequence
  primer
actgtggatattcataaggagaaagtggtccaacccttagggaaccc

SEQ ID NO: 361 - DNA, 49 bp, Artificial Sequence
  primer
gtcaagtacaaaagagattttgaagaaagctccaacccttagggaaccc

SEQ ID NO: 362 - DNA, 50 bp, Artificial Sequence
  primer
gtaaaataccatgaagattttgaaaaaacaatccaacccttagggaaccc

SEQ ID NO: 363 - DNA, 42 bp, Artificial Sequence
  primer
aagcggtaccgcgcggtgtatgatccaacccttagggaaccc

SEQ ID NO: 364 - DNA, 42 bp, Artificial Sequence
  primer
ctggtcagtgagaaggtcggaggtccaacccttagggaaccc

SEQ ID NO: 365 - DNA, 46 bp, Artificial Sequence
  primer
ttctccaatatccccttcttcatcttctccaacccttagggaaccc

SEQ ID NO: 366 - DNA, 42 bp, Artificial Sequence
  primer
ggccctcctcaggacctgtctgttccaacccttagggaaccc

SEQ ID NO: 367 - DNA, 42 bp, Artificial Sequence
  primer
ggcagcaaggagcgcttccactgtccaacccttagggaaccc

SEQ ID NO: 368 - DNA, 43 bp, Artificial Sequence
  primer
acctacatcggctctgtgctcatctccaacccttagggaaccc

SEQ ID NO: 369 - DNA, 42 bp, Artificial Sequence
  primer
accttcacggcatggtgcaactctccaacccttagggaaccc

SEQ ID NO: 370 - DNA, 42 bp, Artificial Sequence
  primer
cctatgggctatgggcctcgtattccaacccttagggaaccc

SEQ ID NO: 371 - DNA, 44 bp, Artificial Sequence
  primer
gcacaaatgtctagttcttcctgcctccaacccttagggaaccc

SEQ ID NO: 372 - DNA, 43 bp, Artificial Sequence
  primer
ctttcccagccagctgtaagcatttccaacccttagggaaccc

SEQ ID NO: 373 - DNA, 47 bp, Artificial Sequence
  primer
gaaacaatgaccgataaaacagagaaggtccaacccttagggaaccc

SEQ ID NO: 374 - DNA, 22 bp, Artificial Sequence
  primer
agcagcagctacgggcagcaga

SEQ ID NO: 375 - DNA, 22 bp, Artificial Sequence
  primer
gaggaggacgcggtggaatggg

SEQ ID NO: 376 - DNA, 24 bp, Artificial Sequence
  primer
gaggtggcttcaataagcctggtg

SEQ ID NO: 377 - DNA, 27 bp, Artificial Sequence
  primer
tggatgaaggaccagatcttgatctag

SEQ ID NO: 378 - DNA, 28 bp, Artificial Sequence
  primer
gttcactgctggcctataatacaacctc

SEQ ID NO: 379 - DNA, 30 bp, Artificial Sequence
  primer
acccttcttatgactcagtcagaagaggag

SEQ ID NO: 380 - DNA, 23 bp, Artificial Sequence
  primer
gtcctccccttggaggggcacaa

SEQ ID NO: 381 - DNA, 26 bp, Artificial Sequence
  primer
gttattccaggatctttggagacccg

SEQ ID NO: 382 - DNA, 28 bp, Artificial Sequence
  primer
gaagccttatcagttgtgagtgaggacc

SEQ ID NO: 383 - DNA, 24 bp, Artificial Sequence
  primer
atttaccatatgagccccccagga

SEQ ID NO: 384 - DNA, 25 bp, Artificial Sequence
  primer
ctgctcaaccatctccttccacagt

SEQ ID NO: 385 - DNA, 30 bp, Artificial Sequence
  primer
atccttatcagattcttggaccaacaagta

SEQ ID NO: 386 - DNA, 22 bp, Artificial Sequence
  primer
gcagtggccagatccagctttg

SEQ ID NO: 387 - DNA, 23 bp, Artificial Sequence
  primer
atcccgtcggagacggtctcttc

SEQ ID NO: 388 - DNA, 28 bp, Artificial Sequence
  primer
ctcaggtacctgacaatgatgagcagtt

SEQ ID NO: 389 - DNA, 29 bp, Artificial Sequence
  primer
gagacatcaaacaagagccaggaatgtat

SEQ ID NO: 390 - DNA, 22 bp, Artificial Sequence
  primer
atgtcaccgggtgcgcatcaat

SEQ ID NO: 391 - DNA, 24 bp, Artificial Sequence
  primer
attggcaatggcctctcacctcag

SEQ ID NO: 392 - DNA, 30 bp, Artificial Sequence
  primer
aattcaattcgtcataatctgtccctacac

SEQ ID NO: 393 - DNA, 29 bp, Artificial Sequence
  primer
gtgagaaaccataccagtgtgacttcaag

SEQ ID NO: 394 - DNA, 23 bp, Artificial Sequence
  primer
cccaggacagcagcagggctacg

SEQ ID NO: 395 - DNA, 26 bp, Artificial Sequence
  primer
agcagaggccttatggatatgaccag

SEQ ID NO: 396 - DNA, 23 bp, Artificial Sequence
  primer
atcatgcccaagaagccagcaga

SEQ ID NO: 397 - DNA, 25 bp, Artificial Sequence
  primer
gtttcaaagtcaccctcccaccttt

SEQ ID NO: 398 - DNA, 30 bp, Artificial Sequence
  primer
tgttcaagaaggaagtgtatcttcatacat

SEQ ID NO: 399 - DNA, 27 bp, Artificial Sequence
  primer
atgtgcttttccagactgatccaactg

SEQ ID NO: 400 - DNA, 23 bp, Artificial Sequence
  primer
tggaggtggaggtggaggtggag

SEQ ID NO: 401 - DNA, 23 bp, Artificial Sequence
  primer
cgtggaggcagaggtggcatggg

SEQ ID NO: 402 - DNA, 25 bp, Artificial Sequence
  primer
cgtggtggcttcaataaatttggtg

SEQ ID NO: 403 - DNA, 24 bp, Artificial Sequence
  primer
accaaggatcacgtcatgactccg

SEQ ID NO: 404 - DNA, 23 bp, Artificial Sequence
  primer
ggctgccgtggaatggtttgatg

SEQ ID NO: 405 - DNA, 28 bp, Artificial Sequence
  primer
cttctttaagcagtgtggggttgttaag

SEQ ID NO: 406 - DNA, 45 bp, Artificial Sequence
  primer
gtgccagcaagatccaatctagaagcagcagctacgggcagcaga

SEQ ID NO: 407 - DNA, 45 bp, Artificial Sequence
  primer
gtgccagcaagatccaatctagagaggaggacgcggtggaatggg

SEQ ID NO: 408 - DNA, 47 bp, Artificial Sequence
  primer
gtgccagcaagatccaatctagagaggtggcttcaataagcctggtg

SEQ ID NO: 409 - DNA, 50 bp, Artificial Sequence
  primer
gtgccagcaagatccaatctagatggatgaaggaccagatcttgatctag

SEQ ID NO: 410 - DNA, 47 bp, Artificial Sequence
  primer
gttcactgctggcctataatacaacctctccaacccttagggaaccc

SEQ ID NO: 411 - DNA, 49 bp, Artificial Sequence
  primer
acccttcttatgactcagtcagaagaggagtccaacccttagggaaccc

SEQ ID NO: 412 - DNA, 42 bp, Artificial Sequence
  primer
gtcctccccttggaggggcacaatccaacccttagggaaccc

SEQ ID NO: 413 - DNA, 45 bp, Artificial Sequence
  primer
gttattccaggatctttggagacccgtccaacccttagggaaccc

SEQ ID NO: 414 - DNA, 47 bp, Artificial Sequence
  primer
gaagccttatcagttgtgagtgaggacctccaacccttagggaaccc

SEQ ID NO: 415 - DNA, 43 bp, Artificial Sequence
  primer
atttaccatatgagccccccaggatccaacccttagggaaccc

SEQ ID NO: 416 - DNA, 44 bp, Artificial Sequence
  primer
ctgctcaaccatctccttccacagttccaacccttagggaaccc

SEQ ID NO: 417 - DNA, 49 bp, Artificial Sequence
  primer
atccttatcagattcttggaccaacaagtatccaacccttagggaaccc

SEQ ID NO: 418 - DNA, 41 bp, Artificial Sequence
  primer
gcagtggccagatccagctttgtccaacccttagggaaccc

SEQ ID NO: 419 - DNA, 42 bp, Artificial Sequence
  primer
atcccgtcggagacggtctcttctccaacccttagggaaccc

SEQ ID NO: 420 - DNA, 47 bp, Artificial Sequence
  primer
ctcaggtacctgacaatgatgagcagtttccaacccttagggaaccc

SEQ ID NO: 421 - DNA, 48 bp, Artificial Sequence
  primer
gagacatcaaacaagagccaggaatgtattccaacccttagggaaccc

SEQ ID NO: 422 - DNA, 41 bp, Artificial Sequence
  primer
atgtcaccgggtgcgcatcaattccaacccttagggaaccc

SEQ ID NO: 423 - DNA, 47 bp, Artificial Sequence
  primer
gtgccagcaagatccaatctagaattggcaatggcctctcacctcag

SEQ ID NO: 424 - DNA, 49 bp, Artificial Sequence
  primer
aattcaattcgtcataatctgtccctacactccaacccttagggaaccc

SEQ ID NO: 425 - DNA, 48 bp, Artificial Sequence
  primer
gtgagaaaccataccagtgtgacttcaagtccaacccttagggaaccc

SEQ ID NO: 426 - DNA, 46 bp, Artificial Sequence
  primer
gtgccagcaagatccaatctagacccaggacagcagcagggctacg

SEQ ID NO: 427 - DNA, 49 bp, Artificial Sequence
  primer
gtgccagcaagatccaatctagaagcagaggccttatggatatgaccag

SEQ ID NO: 428 - DNA, 42 bp, Artificial Sequence
  primer
atcatgcccaagaagccagcagatccaacccttagggaaccc

SEQ ID NO: 429 - DNA, 44 bp, Artificial Sequence
  primer
gtttcaaagtcaccctcccaccttttccaacccttagggaaccc

SEQ ID NO: 430 - DNA, 49 bp, Artificial Sequence
  primer
tgttcaagaaggaagtgtatcttcatacattccaacccttagggaaccc

SEQ ID NO: 431 - DNA, 46 bp, Artificial Sequence
  primer
atgtgcttttccagactgatccaactgtccaacccttagggaaccc

SEQ ID NO: 432 - DNA, 46 bp, Artificial Sequence
  primer
gtgccagcaagatccaatctagatggaggtggaggtggaggtggag

SEQ ID NO: 433 - DNA, 46 bp, Artificial Sequence
  primer
gtgccagcaagatccaatctagacgtggaggcagaggtggcatggg

SEQ ID NO: 434 - DNA, 48 bp, Artificial Sequence
  primer
gtgccagcaagatccaatctagacgtggtggcttcaataaatttggtg

SEQ ID NO: 435 - DNA, 47 bp, Artificial Sequence
  primer
gtgccagcaagatccaatctagaaccaaggatcacgtcatgactccg

SEQ ID NO: 436 - DNA, 46 bp, Artificial Sequence
  primer
gtgccagcaagatccaatctagaggctgccgtggaatggtttgatg

SEQ ID NO: 437 - DNA, 51 bp, Artificial Sequence
  primer
gtgccagcaagatccaatctagacttctttaagcagtgtggggttgttaag

SEQ ID NO: 438 - DNA, 25 bp, Artificial Sequence
  primer
acatttcatggggctccactaacag

SEQ ID NO: 439 - DNA, 23 bp, Artificial Sequence
  primer
gctgcctgcgtcccaaagaacag

SEQ ID NO: 440 - DNA, 27 bp, Artificial Sequence
  primer
ctacagagacacaacccattgtttatg

SEQ ID NO: 441 - DNA, 27 bp, Artificial Sequence
  primer
gtgggaacgtgaaacatctgatacaag

SEQ ID NO: 442 - DNA, 24 bp, Artificial Sequence
  primer
ggaagattgcccgagagcaaaaag

SEQ ID NO: 443 - DNA, 27 bp, Artificial Sequence
  primer
tgcatattagtggacagcacttagtag

SEQ ID NO: 444 - DNA, 23 bp, Artificial Sequence
  primer
gcagcccaagcttcccatcacag

SEQ ID NO: 445 - DNA, 27 bp, Artificial Sequence
  primer
gaccttccaccaatattcctgaaaatg

SEQ ID NO: 446 - DNA, 27 bp, Artificial Sequence
  primer
ttggcttaacagatgatcaggtttcag

SEQ ID NO: 447 - DNA, 27 bp, Artificial Sequence
  primer
ctcagactcaagcaggtcagattgaag

SEQ ID NO: 448 - DNA, 27 bp, Artificial Sequence
  primer
ctcaacagtatggtattcagtattcag

SEQ ID NO: 449 - DNA, 23 bp, Artificial Sequence
  primer
atatgccctgcgtccaagcccaa

SEQ ID NO: 450 - DNA, 23 bp, Artificial Sequence
  primer
agcccactgcggaagagggcagc

SEQ ID NO: 451 - DNA, 30 bp, Artificial Sequence
  primer
ctgcatcaggagatatgcaaacatatcaga

SEQ ID NO: 452 - DNA, 22 bp, Artificial Sequence
  primer
ttgccattgccccaaatggagc

SEQ ID NO: 453 - DNA, 30 bp, Artificial Sequence
  primer
aaaaattttgaaagacttatcttctgaaga

SEQ ID NO: 454 - DNA, 30 bp, Artificial Sequence
  primer
gtatcatctttatcagaaagtgaggagtcc

SEQ ID NO: 455 - DNA, 23 bp, Artificial Sequence
  primer
ttgccattacccagggaggagca

SEQ ID NO: 456 - DNA, 27 bp, Artificial Sequence
  primer
ctgcctctggagacgtacaaacatacc

SEQ ID NO: 457 - DNA, 28 bp, Artificial Sequence
  primer
atgtgcagcacattaagaggagagacat

SEQ ID NO: 458 - DNA, 23 bp, Artificial Sequence
  primer
gtgcagacccatctggagaaccc

SEQ ID NO: 459 - DNA, 29 bp, Artificial Sequence
  primer
attgatgatgtcattgatgagatcatcag

SEQ ID NO: 460 - DNA, 24 bp, Artificial Sequence
  primer
ctgcctgtgtcagggaatctgctt

SEQ ID NO: 461 - DNA, 26 bp, Artificial Sequence
  primer
ggaaaactacagccaccacacacaag

SEQ ID NO: 462 - DNA, 28 bp, Artificial Sequence
  primer
acacctttcatgaactcaaatctgatgg

SEQ ID NO: 463 - DNA, 29 bp, Artificial Sequence
  primer
ggtcatactgcatcagaaccatgaagaag

SEQ ID NO: 464 - DNA, 24 bp, Artificial Sequence
  primer
ccatgcccattgggagaatagcag

SEQ ID NO: 465 - DNA, 23 bp, Artificial Sequence
  primer
agcaggagcaggagcgggagcgg

SEQ ID NO: 466 - DNA, 23 bp, Artificial Sequence
  primer
cgccggagttgcataagggagat

SEQ ID NO: 467 - DNA, 24 bp, Artificial Sequence
  primer
ggttccatgatgggaagtgacatg

SEQ ID NO: 468 - DNA, 28 bp, Artificial Sequence
  primer
tggccaatgtgatctggaacttattaat

SEQ ID NO: 469 - DNA, 23 bp, Artificial Sequence
  primer
tcaagggaaccttccctgatgcg

SEQ ID NO: 470 - DNA, 27 bp, Artificial Sequence
  primer
cagtgatctggcctcagacaactactg

SEQ ID NO: 471 - DNA, 29 bp, Artificial Sequence
  primer
gcataagctggaagtcacaccagtagtag

SEQ ID NO: 472 - DNA, 27 bp, Artificial Sequence
  primer
aggttgaaattgggtcttcaaaaccag

SEQ ID NO: 473 - DNA, 23 bp, Artificial Sequence
  primer
tcaggatgggaaaattgcaccag

SEQ ID NO: 474 - DNA, 35 bp, Artificial Sequence
  primer
gagaatatataaaaaactggaggccaagatacttc

SEQ ID NO: 475 - DNA, 30 bp, Artificial Sequence; identical to SEQ ID NO: 77
  primer

SEQ ID NO: 476 - DNA, 30 bp, Artificial Sequence; identical to SEQ ID NO: 78
  primer

SEQ ID NO: 477 - DNA, 30 bp, Artificial Sequence; identical to SEQ ID NO: 451
  primer

SEQ ID NO: 478 - DNA, 22 bp, Artificial Sequence
  primer
ttgccattgccccaaatggagc

SEQ ID NO: 479 - DNA, 30 bp, Artificial Sequence; identical to SEQ ID NO: 453
  primer

SEQ ID NO: 480 - DNA, 30 bp, Artificial Sequence; identical to SEQ ID NO: 454
  primer

SEQ ID NO: 481 - DNA, 48 bp, Artificial Sequence
  primer
gtgccagcaagatccaatctagaacatttcatggggctccactaacag

SEQ ID NO: 482 - DNA, 46 bp, Artificial Sequence
  primer
gtgccagcaagatccaatctagagctgcctgcgtcccaaagaacag

SEQ ID NO: 483 - DNA, 50 bp, Artificial Sequence
  primer
gtgccagcaagatccaatctagactacagagacacaacccattgtttatg

SEQ ID NO: 484 - DNA, 50 bp, Artificial Sequence
  primer
gtgccagcaagatccaatctagagtgggaacgtgaaacatctgatacaag

SEQ ID NO: 485 - DNA, 47 bp, Artificial Sequence
  primer
gtgccagcaagatccaatctagaggaagattgcccgagagcaaaaag

SEQ ID NO: 486 - DNA, 50 bp, Artificial Sequence
  primer
gtgccagcaagatccaatctagatgcatattagtggacagcacttagtag

SEQ ID NO: 487 - DNA, 46 bp, Artificial Sequence
  primer
gtgccagcaagatccaatctagagcagcccaagcttcccatcacag

SEQ ID NO: 488 - DNA, 50 bp, Artificial Sequence
  primer
gtgccagcaagatccaatctagagaccttccaccaatattcctgaaaatg

SEQ ID NO: 489 - DNA, 50 bp, Artificial Sequence
  primer
gtgccagcaagatccaatctagattggcttaacagatgatcaggtttcag

SEQ ID NO: 490 - DNA, 50 bp, Artificial Sequence
  primer
gtgccagcaagatccaatctagactcagactcaagcaggtcagattgaag

SEQ ID NO: 491 - DNA, 50 bp, Artificial Sequence
  primer
gtgccagcaagatccaatctagactcaacagtatggtattcagtattcag

SEQ ID NO: 492 - DNA, 42 bp, Artificial Sequence
  primer
atatgccctgcgtccaagcccaatccaacccttagggaaccc

SEQ ID NO: 493 - DNA, 42 bp, Artificial Sequence
  primer
agcccactgcggaagagggcagctccaacccttagggaaccc

SEQ ID NO: 494 - DNA, 49 bp, Artificial Sequence
  primer
ctgcatcaggagatatgcaaacatatcagatccaacccttagggaaccc

SEQ ID NO: 495 - DNA, 41 bp, Artificial Sequence
  primer
ttgccattgccccaaatggagctccaacccttagggaaccc

SEQ ID NO: 496 - DNA, 49 bp, Artificial Sequence
  primer
aaaaattttgaaagacttatcttctgaagatccaacccttagggaaccc

SEQ ID NO: 497 - DNA, 49 bp, Artificial Sequence
  primer
gtatcatctttatcagaaagtgaggagtcctccaacccttagggaaccc

SEQ ID NO: 498 - DNA, 42 bp, Artificial Sequence
  primer
ttgccattacccagggaggagcatccaacccttagggaaccc

SEQ ID NO: 499 - DNA, 46 bp, Artificial Sequence
  primer
ctgcctctggagacgtacaaacatacctccaacccttagggaaccc

SEQ ID NO: 500 - DNA, 47 bp, Artificial Sequence
  primer
atgtgcagcacattaagaggagagacattccaacccttagggaaccc

SEQ ID NO: 501 - DNA, 42 bp, Artificial Sequence
  primer
gtgcagacccatctggagaaccctccaacccttagggaaccc

SEQ ID NO: 502 - DNA, 48 bp, Artificial Sequence
  primer
attgatgatgtcattgatgagatcatcagtccaacccttagggaaccc

SEQ ID NO: 503 - DNA, 43 bp, Artificial Sequence
  primer
ctgcctgtgtcagggaatctgctttccaacccttagggaaccc

SEQ ID NO: 504 - DNA, 49 bp, Artificial Sequence
  primer
gtgccagcaagatccaatctagaggaaaactacagccaccacacacaag

SEQ ID NO: 505 - DNA, 51 bp, Artificial Sequence
  primer
gtgccagcaagatccaatctagaacacctttcatgaactcaaatctgatgg

SEQ ID NO: 506 - DNA, 52 bp, Artificial Sequence; identical to SEQ ID NO: 56
  primer

SEQ ID NO: 507 - DNA, 47 bp, Artificial Sequence; identical to SEQ ID NO: 57
  primer

SEQ ID NO: 508 - DNA, 46 bp, Artificial Sequence
  primer
gtgccagcaagatccaatctagaagcaggagcaggagcgggagcgg

SEQ ID NO: 509 - DNA, 46 bp, Artificial Sequence
  primer
gtgccagcaagatccaatctagacgccggagttgcataagggagat

SEQ ID NO: 510 - DNA, 47 bp, Artificial Sequence
  primer
gtgccagcaagatccaatctagaggttccatgatgggaagtgacatg

SEQ ID NO: 511 - DNA, 51 bp, Artificial Sequence
  primer
gtgccagcaagatccaatctagatggccaatgtgatctggaacttattaat

SEQ ID NO: 512 - DNA, 46 bp, Artificial Sequence
  primer
gtgccagcaagatccaatctagatcaagggaaccttccctgatgcg

SEQ ID NO: 513 - DNA, 50 bp, Artificial Sequence
  primer
gtgccagcaagatccaatctagacagtgatctggcctcagacaactactg

SEQ ID NO: 514 - DNA, 48 bp, Artificial Sequence
  primer
gcataagctggaagtcacaccagtagtagtccaacccttagggaaccc

SEQ ID NO: 515 - DNA, 50 bp, Artificial Sequence
  primer
gtgccagcaagatccaatctagaaggttgaaattgggtcttcaaaaccag

SEQ ID NO: 516 - DNA, 46 bp, Artificial Sequence
  primer
gtgccagcaagatccaatctagatcaggatgggaaaattgcaccag

SEQ ID NO: 517 - DNA, 54 bp, Artificial Sequence
  primer
gagaatatataaaaaactggaggccaagatacttctccaacccttagggaaccc

SEQ ID NO: 518 - DNA, 53 bp, Artificial Sequence; identical to SEQ ID NO: 231
  primer

SEQ ID NO: 519 - DNA, 53 bp, Artificial Sequence; identical to SEQ ID NO: 232
  primer

SEQ ID NO: 520 - DNA, 49 bp, Artificial Sequence; identical to SEQ ID NO: 494
  primer

SEQ ID NO: 521 - DNA, 41 bp, Artificial Sequence; identical to SEQ ID NO: 495
  primer

SEQ ID NO: 522 - DNA, 49 bp, Artificial Sequence; identical to SEQ ID NO: 496
  primer

SEQ ID NO: 523 - DNA, 49 bp, Artificial Sequence; identical to SEQ ID NO: 497
  primer

SEQ ID NO: 524 - DNA, 26 bp, Artificial Sequence
  primer
gttattccaggatctttggagacccg

SEQ ID NO: 525 - DNA, 28 bp, Artificial Sequence
  primer
gaagccttatcagttgtgagtgaggacc

SEQ ID NO: 526 - DNA, 24 bp, Artificial Sequence
  primer
atttaccatatgagccccccagga

SEQ ID NO: 527 - DNA, 25 bp, Artificial Sequence
  primer
ctgctcaaccatctccttccacagt

SEQ ID NO: 528 - DNA, 30 bp, Artificial Sequence; identical to SEQ ID NO: 385
  primer

SEQ ID NO: 529 - DNA, 22 bp, Artificial Sequence
  primer
gcagtggccagatccagctttg

SEQ ID NO: 530 - DNA, 22 bp, Artificial Sequence
  primer
agcgccgcctggagcgcggcag

SEQ ID NO: 531 - DNA, 27 bp, Artificial Sequence
  primer
gataacagcaagatggctttgaactca

SEQ ID NO: 532 - DNA, 23 bp, Artificial Sequence
  primer
tgtaccgccggaagcaccaggag

SEQ ID NO: 533 - DNA, 23 bp, Artificial Sequence
  primer
ggctggaaacatttccgaccctg

SEQ ID NO: 534 - DNA, 27 bp, Artificial Sequence
  primer
tggaaaagacaattgatgacctggaag

SEQ ID NO: 535 - DNA, 27 bp, Artificial Sequence
  primer
cagtgaaaaaatcagtctcaagtaaag

SEQ ID NO: 536 - DNA, 27 bp, Artificial Sequence
  primer
agcataaagatgtcatcatcaaccaag

SEQ ID NO: 537 - DNA, 25 bp, Artificial Sequence
  primer
cccacacctgggaaaggacctaaag

SEQ ID NO: 538 - DNA, 27 bp, Artificial Sequence
  primer
gatctgaatcctgaaagagaaatagag

SEQ ID NO: 539 - DNA, 23 bp, Artificial Sequence
  primer
gccataggaacgcactcaggcag

SEQ ID NO: 540 - DNA, 27 bp, Artificial Sequence
  primer
agctctctgtgatgcgctactcaatag

SEQ ID NO: 541 - DNA, 27 bp, Artificial Sequence
  primer
actcgggagactatgaaatattgtact

SEQ ID NO: 542 - DNA, 25 bp, Artificial Sequence
  primer
ctggagtcccaaataaaccaggcat

SEQ ID NO: 543 - DNA, 30 bp, Artificial Sequence
  primer
atgatttttggataccagaaacaagtttca

SEQ ID NO: 544 - DNA, 30 bp, Artificial Sequence
  primer
tctggcatagaagattaaagaatcaaaaaa

SEQ ID NO: 545 - DNA, 24 bp, Artificial Sequence
  primer
agctgtctggctctggagatctgg

SEQ ID NO: 546 - DNA, 23 bp, Artificial Sequence
  primer
tgagagaacggaggtcctggcag

SEQ ID NO: 547 - DNA, 27 bp, Artificial Sequence
  primer
acataaccattagcagagaggctcagg

SEQ ID NO: 548 - DNA, 23 bp, Artificial Sequence
  primer
gcccactgacgctccaccgaaag

SEQ ID NO: 549 - DNA, 26 bp, Artificial Sequence
  primer
ggagaagacaaagaaggcagagagag

SEQ ID NO: 550 - DNA, 27 bp, Artificial Sequence
  primer
ttttcttaccacaacatgacagtagtg

SEQ ID NO: 551 - DNA, 23 bp, Artificial Sequence
  primer
atccactgtgcgacgagctgtgc

SEQ ID NO: 552 - DNA, 24 bp, Artificial Sequence
  primer
gaggatccaaagtgggaattccct

SEQ ID NO: 553 - DNA, 27 bp, Artificial Sequence
  primer
attgctgtgggaaataatgatgtaaag

SEQ ID NO: 554 - DNA, 27 bp, Artificial Sequence
  primer
gcagcatgtcagcttcgtatctctcaa

SEQ ID NO: 555 - DNA, 27 bp, Artificial Sequence
  primer
aagaactagtccagcttcgagcacaag

SEQ ID NO: 556 - DNA, 27 bp, Artificial Sequence
  primer
caggacctggctacaagagttaaaaag

SEQ ID NO: 557 - DNA, 27 bp, Artificial Sequence
  primer
gaacagctcactaaagtgcacaaacag

SEQ ID NO: 558 - DNA, 25 bp, Artificial Sequence
  primer
agaagagggcattctgcacagattg

SEQ ID NO: 559 - DNA, 23 bp, Artificial Sequence
  primer
tgcgcaaagccagcgtgaccatc

SEQ ID NO: 560 - DNA, 45 bp, Artificial Sequence; identical to SEQ ID NO: 413
  primer

SEQ ID NO: 561 - DNA, 47 bp, Artificial Sequence; identical to SEQ ID NO: 414
  primer

SEQ ID NO: 562 - DNA, 43 bp, Artificial Sequence; identical to SEQ ID NO: 415
  primer

SEQ ID NO: 563 - DNA, 44 bp, Artificial Sequence; identical to SEQ ID NO: 416
  primer

SEQ ID NO: 564 - DNA, 49 bp, Artificial Sequence; identical to SEQ ID NO: 417
  primer

SEQ ID NO: 565 - DNA, 41 bp, Artificial Sequence; identical to SEQ ID NO: 418
  primer

SEQ ID NO: 566 - DNA, 45 bp, Artificial Sequence
  primer
gtgccagcaagatccaatctagaagcgccgcctggagcgcggcag

SEQ ID NO: 567 - DNA, 50 bp, Artificial Sequence
  primer
gtgccagcaagatccaatctagagataacagcaagatggctttgaactca

SEQ ID NO: 568 - DNA, 42 bp, Artificial Sequence
  primer
tgtaccgccggaagcaccaggagtccaacccttagggaaccc

SEQ ID NO: 569 - DNA, 46 bp, Artificial Sequence
  primer
gtgccagcaagatccaatctagaggctggaaacatttccgaccctg

SEQ ID NO: 570 - DNA, 50 bp, Artificial Sequence
  primer
gtgccagcaagatccaatctagatggaaaagacaattgatgacctggaag

SEQ ID NO: 571 - DNA, 50 bp, Artificial Sequence
  primer
gtgccagcaagatccaatctagacagtgaaaaaatcagtctcaagtaaag

SEQ ID NO: 572 - DNA, 50 bp, Artificial Sequence
  primer
gtgccagcaagatccaatctagaagcataaagatgtcatcatcaaccaag

SEQ ID NO: 573 - DNA, 48 bp, Artificial Sequence
  primer
gtgccagcaagatccaatctagacccacacctgggaaaggacctaaag

SEQ ID NO: 574 - DNA, 50 bp, Artificial Sequence
  primer
gtgccagcaagatccaatctagagatctgaatcctgaaagagaaatagag

SEQ ID NO: 575 - DNA, 46 bp, Artificial Sequence
  primer
gtgccagcaagatccaatctagagccataggaacgcactcaggcag

SEQ ID NO: 576 - DNA, 50 bp, Artificial Sequence
  primer
gtgccagcaagatccaatctagaagctctctgtgatgcgctactcaatag

SEQ ID NO: 577 - DNA, 50 bp, Artificial Sequence
  primer
gtgccagcaagatccaatctagaactcgggagactatgaaatattgtact

SEQ ID NO: 578 - DNA, 44 bp, Artificial Sequence
  primer
ctggagtcccaaataaaccaggcattccaacccttagggaaccc

SEQ ID NO: 579 - DNA, 49 bp, Artificial Sequence
  primer
atgatttttggataccagaaacaagtttcatccaacccttagggaaccc

SEQ ID NO: 580 - DNA, 49 bp, Artificial Sequence
  primer
tctggcatagaagattaaagaatcaaaaaatccaacccttagggaaccc

SEQ ID NO: 581 - DNA, 46 bp, Artificial Sequence
  primer
gtgccagcaagatccaatctagaagctgtctggctctggagatctg

SEQ ID NO: 582 - DNA, 46 bp, Artificial Sequence
  primer
gtgccagcaagatccaatctagatgagagaacggaggtcctggcag

SEQ ID NO: 583 - DNA, 50 bp, Artificial Sequence
  primer
gtgccagcaagatccaatctagaacataaccattagcagagaggctcagg

SEQ ID NO: 584 - DNA, 46 bp, Artificial Sequence
  primer
gtgccagcaagatccaatctagagcccactgacgctccaccgaaag

SEQ ID NO: 585 - DNA, 49 bp, Artificial Sequence
  primer
gtgccagcaagatccaatctagaggagaagacaaagaaggcagagagag

SEQ ID NO: 586 - DNA, 50 bp, Artificial Sequence
  primer
gtgccagcaagatccaatctagattttcttaccacaacatgacagtagtg

SEQ ID NO: 587 - DNA, 42 bp, Artificial Sequence
  primer
atccactgtgcgacgagctgtgctccaacccttagggaaccc

SEQ ID NO: 588 - DNA, 43 bp, Artificial Sequence
  primer
gaggatccaaagtgggaattcccttccaacccttagggaaccc

SEQ ID NO: 589 - DNA, 50 bp, Artificial Sequence
  primer
gtgccagcaagatccaatctagaattgctgtgggaaataatgatgtaaag

SEQ ID NO: 590 - DNA, 50 bp, Artificial Sequence
  primer
gtgccagcaagatccaatctagagcagcatgtcagcttcgtatctctcaa

SEQ ID NO: 591 - DNA, 50 bp, Artificial Sequence
  primer
gtgccagcaagatccaatctagaaagaactagtccagcttcgagcacaag

SEQ ID NO: 592 - DNA, 50 bp, Artificial Sequence
  primer
gtgccagcaagatccaatctagacaggacctggctacaagagttaaaaag

SEQ ID NO: 593 - DNA, 50 bp, Artificial Sequence
  primer
gtgccagcaagatccaatctagagaacagctcactaaagtgcacaaacag

SEQ ID NO: 594 - DNA, 48 bp, Artificial Sequence
  primer
gtgccagcaagatccaatctagaagaagagggcattctgcacagattg

SEQ ID NO: 595 - DNA, 46 bp, Artificial Sequence
  primer
gtgccagcaagatccaatctagatgcgcaaagccagcgtgaccatc

SEQ ID NO: 596 - DNA, 21 bp, Artificial Sequence
  primer forward
cgcaaatgggcggtaggcgtg

SEQ ID NO: 597 - DNA, 19 bp, Artificial Sequence
  primer reverse
cgccatccacgctgttttg

SEQ ID NO: 598 - DNA, 21 bp, Artificial Sequence
  primer forward
ggctaactagagaacccactg

SEQ ID NO: 599 - DNA, 20 bp, Artificial Sequence
  primer reverse
ggcaactagaaggcacagtc

SEQ ID NO: 600 - DNA, 20 bp, Artificial Sequence
  primer forward
agagctcgtttagtgaaccg

SEQ ID NO: 601 - DNA, 20 bp, Artificial Sequence
  primer reverse
gtggtttgtccaaactcatc

SEQ ID NO: 602 - DNA, 19 bp, Artificial Sequence
  primer forward
gatcactctcggcatggac

SEQ ID NO: 603 - DNA, 23 bp, Artificial Sequence
  primer reverse
cattttatgtttcaggttcaggg

SEQ ID NO: 604 - DNA, 18 bp, Artificial Sequence
  primer forward
gtcgtaacaactccgccc

SEQ ID NO: 605 - DNA, 19 bp, Artificial Sequence
  primer reverse
gtccagctcgaccaggatg

SEQ ID NO: 606 - DNA, 19 bp, Artificial Sequence
  primer forward
atagcatggcctttgcagg

SEQ ID NO: 607 - DNA, 19 bp, Artificial Sequence
  primer reverse
gagctgcatgtgtcagagg

SEQ ID NO: 608 - DNA, 22 bp, Artificial Sequence
  primer forward
gtatcttatggtactgtaactg

SEQ ID NO: 609 - DNA, 22 bp, Artificial Sequence
  primer reverse
ctttatgtttttggcgtcttcc

SEQ ID NO: 610 - DNA, 20 bp, Artificial Sequence
  primer forward
ggtctatataagcagagctg

SEQ ID NO: 611 - DNA, 23 bp, Artificial Sequence
  primer reverse
gtggtatggctgattatgatcag

SEQ ID NO: 612 - DNA, 26 bp, Artificial Sequence
  AF6 exon 2
ctcatcactccatggaactccaaatc

SEQ ID NO: 613 - DNA, 24 bp, Artificial Sequence
  MLL exon 9
cttttcttttggtttttgttttac

SEQ ID NO: 614 - DNA, 23 bp, Artificial Sequence
  AF1Q exon 2
ctcacagggtccctcatagcttc

SEQ ID NO: 615 - DNA, 13 bp, Artificial Sequence
  MLL exon 10
cttaaagtccact

SEQ ID NO: 616 - DNA, 23 bp, Artificial Sequence
  primer
ctctacgtctcctccgagagccg

SEQ ID NO: 617 - DNA, 23 bp, Artificial Sequence
  primer
gtctcctcctcggcttcctcttc

SEQ ID NO: 618 - DNA, 29 bp, Artificial Sequence
  primer
ataatctcagtgataccttgaagaagctg

SEQ ID NO: 619 - DNA, 28 bp, Artificial Sequence
  primer
aaatgacatcagatgtaccatcactggg

SEQ ID NO: 620 - DNA, 29 bp, Artificial Sequence
  primer
tcgcttcatggagatataagtagcctgaa

SEQ ID NO: 621 - DNA, 27 bp, Artificial Sequence
  primer
gaaattcggcgccttcatcagtatgtg

SEQ ID NO: 622 - DNA, 30 bp, Artificial Sequence
  primer
atcgtactgagaagcactccacaatgccag

SEQ ID NO: 623 - DNA, 33 bp, Artificial Sequence
  primer
caggaacgaatttcatatacacctccagagagc

SEQ ID NO: 624 - DNA, 26 bp, Artificial Sequence
  primer
gcttgcagccaatttactggagcagg

SEQ ID NO: 625 - DNA, 33 bp, Artificial Sequence
  primer
atcttagacgaattttacaatgtgaagttctgc

SEQ ID NO: 626 - DNA, 24 bp, Artificial Sequence
  primer
gtgtctgctgactccagtgcatcc

SEQ ID NO: 627 - DNA, 28 bp, Artificial Sequence
  primer
attgtcagtaaacgggcaggtactcagt

SEQ ID NO: 628 - DNA, 27 bp, Artificial Sequence
  primer
gatgacagcatggaagagaaaccacta

SEQ ID NO: 629 - DNA, 23 bp, Artificial Sequence
  primer
tgagtccattcttgcacaccgag

SEQ ID NO: 630 - DNA, 27 bp, Artificial Sequence
  primer
attttggctctctatttgacttggagc

SEQ ID NO: 631 - DNA, 24 bp, Artificial Sequence
  primer
atgggaatgatgaacaaccccaat

SEQ ID NO: 632 - DNA, 23 bp, Artificial Sequence
  primer
ccttgccctttaaggtggtggtg

SEQ ID NO: 633 - DNA, 28 bp, Artificial Sequence
  primer
ctgccttatgactcaagatgggagtttc

SEQ ID NO: 634 - DNA, 22 bp, Artificial Sequence
  primer
gggccccgagaacctcgaaatc

SEQ ID NO: 635 - DNA, 22 bp, Artificial Sequence
  primer
gggcgacctcttccagaagctg

SEQ ID NO: 636 - DNA, 27 bp, Artificial Sequence
  primer
atccaacgaccaagaactctctggaaa

SEQ ID NO: 637 - DNA, 28 bp, Artificial Sequence
  primer
gccagttatttgcctcagtaaaagacag

SEQ ID NO: 638 - DNA, 30 bp, Artificial Sequence
  primer
cagtatgagtacacggagctcaagaaacag

SEQ ID NO: 639 - DNA, 28 bp, Artificial Sequence
  primer
cacaatgaaacagatttgcaaaaaggaa

SEQ ID NO: 640 - DNA, 20 bp, Artificial Sequence
  primer
ccgcctgcctgcgcacctgc

SEQ ID NO: 641 - DNA, 22 bp, Artificial Sequence
  primer
cgggcagaaggctggtgacaag

SEQ ID NO: 642 - DNA, 20 bp, Artificial Sequence
  primer
aagggccaaccactggggaa

SEQ ID NO: 643 - DNA, 30 bp, Artificial Sequence
  primer
aaaacaagtgcacagacaacaccaagtaag

SEQ ID NO: 644 - DNA, 26 bp, Artificial Sequence
  primer
gggcaaatgagaacagcaacatacag

SEQ ID NO: 645 - DNA, 26 bp, Artificial Sequence
  primer
gcacacctctcaatgcagctttacag

SEQ ID NO: 646 - DNA, 29 bp, Artificial Sequence
  primer
tgaggacaagttctacagccacaagaaaa

SEQ ID NO: 647 - DNA, 24 bp, Artificial Sequence
  primer
agaaccccaacagcaaagaaggct

SEQ ID NO: 648 - DNA, 25 bp, Artificial Sequence
  primer
cctaagatgcccgacttcaactgct

SEQ ID NO: 649 - DNA, 24 bp, Artificial Sequence
  primer
aatgctggacttccgtagtgacca

SEQ ID NO: 650 - DNA, 30 bp, Artificial Sequence
  primer
cagtgccaggaaagagaattagagatcagt

SEQ ID NO: 651 - DNA, 23 bp, Artificial Sequence
  primer
caaagcccacgctgaagcgaaag

SEQ ID NO: 652 - DNA, 26 bp, Artificial Sequence
  primer
agctaaaaggacagcaggtgctacca

SEQ ID NO: 653 - DNA, 28 bp, Artificial Sequence
  primer
gttcagagactgaaggatgaagccagag

SEQ ID NO: 654 - DNA, 29 bp, Artificial Sequence
  primer
gatgatgatgaagaggatgatgatgaaga

SEQ ID NO: 655 - DNA, 24 bp, Artificial Sequence
  primer
cagcttccagtcacagcataggct

SEQ ID NO: 656 - DNA, 28 bp, Artificial Sequence
  primer
gatgtcagcatacccagatccacattag

SEQ ID NO: 657 - DNA, 24 bp, Artificial Sequence
  primer
accgcttggaatgctgcaacaatg

SEQ ID NO: 658 - DNA, 27 bp, Artificial Sequence
  primer
ccaacaggcatgataggatatggaatt

SEQ ID NO: 659 - DNA, 25 bp, Artificial Sequence
  primer
ctttggccctgtatcaggagcacag

SEQ ID NO: 660 - DNA, 21 bp, Artificial Sequence
  primer
ctgggccagaatggtgaggag

SEQ ID NO: 661 - DNA, 23 bp, Artificial Sequence
  primer
ggctggcgcacctgggcgtgcag

SEQ ID NO: 662 - DNA, 25 bp, Artificial Sequence
  primer
tgatgaaggggaggaaggagaggaa

SEQ ID NO: 663 - DNA, 28 bp, Artificial Sequence
  primer
cgtgatctttgatagatccagggaagag

SEQ ID NO: 664 - DNA, 27 bp, Artificial Sequence
  primer
ttcttggctcaacaagccataaaacag

SEQ ID NO: 665 - DNA, 29 bp, Artificial Sequence
  primer
ggtcttataatcttccctctcttccggat

SEQ ID NO: 666 - DNA, 25 bp, Artificial Sequence
  primer
ccagcaaccttccatctctcatcag

SEQ ID NO: 667 - DNA, 24 bp, Artificial Sequence
  primer
ccgcagagcactgtatcacgaaaa

SEQ ID NO: 668 - DNA, 30 bp, Artificial Sequence
  primer
attcctgttcctactacagttcctgttcct

SEQ ID NO: 669 - DNA, 29 bp, Artificial Sequence
  primer
accaagaggctattcaagatctctgcctg

SEQ ID NO: 670 - DNA, 29 bp, Artificial Sequence
  primer
accaagaggctattcaagatctctgtatg

SEQ ID NO: 671 - DNA, 30 bp, Artificial Sequence
  primer
agcatttggttttaaattatggagtatgtt

SEQ ID NO: 672 - DNA, 26 bp, Artificial Sequence
  primer
tctgtggagacgagaatattctggtt

SEQ ID NO: 673 - DNA, 27 bp, Artificial Sequence
  primer
gatttgtgattttggtctagccagagt

SEQ ID NO: 674 - DNA, 31 bp, Artificial Sequence
  primer
catcaagaatgattctaattatgtggttaaa

SEQ ID NO: 675 - DNA, 42 bp, Artificial Sequence
  primer
ctctacgtctcctccgagagccgtccaacccttagggaaccc

SEQ ID NO: 676 - DNA, 42 bp, Artificial Sequence; identical to SEQ ID NO: 298
  primer

SEQ ID NO: 677 - DNA, 48 bp, Artificial Sequence; identical to SEQ ID NO: 256
  primer

SEQ ID NO: 678 - DNA, 47 bp, Artificial Sequence
  primer
aaatgacatcagatgtaccatcactgggtccaacccttagggaaccc

SEQ ID NO: 679 - DNA, 48 bp, Artificial Sequence
  primer
tcgcttcatggagatataagtagcctgaatccaacccttagggaaccc

SEQ ID NO: 680 - DNA, 46 bp, Artificial Sequence
  primer
gaaattcggcgccttcatcagtatgtgtccaacccttagggaaccc

SEQ ID NO: 681 - DNA, 49 bp, Artificial Sequence
  primer
atcgtactgagaagcactccacaatgccagtccaacccttagggaaccc

SEQ ID NO: 682 - DNA, 52 bp, Artificial Sequence
  primer
caggaacgaatttcatatacacctccagagagctccaacccttagggaaccc

SEQ ID NO: 683 - DNA, 45 bp, Artificial Sequence
  primer
gcttgcagccaatttactggagcaggtccaacccttagggaaccc

SEQ ID NO: 684 - DNA, 52 bp, Artificial Sequence
  primer
atcttagacgaattttacaatgtgaagttctgctccaacccttagggaaccc

SEQ ID NO: 685 - DNA, 43 bp, Artificial Sequence
  primer
gtgtctgctgactccagtgcatcctccaacccttagggaaccc

SEQ ID NO: 686 - DNA, 47 bp, Artificial Sequence
  primer
attgtcagtaaacgggcaggtactcagttccaacccttagggaaccc

SEQ ID NO: 687 - DNA, 46 bp, Artificial Sequence
  primer
gatgacagcatggaagagaaaccactatccaacccttagggaaccc

SEQ ID NO: 688 - DNA, 42 bp, Artificial Sequence
  primer
tgagtccattcttgcacaccgagtccaacccttagggaaccc

SEQ ID NO: 689 - DNA, 46 bp, Artificial Sequence
  primer
attttggctctctatttgacttggagctccaacccttagggaaccc

SEQ ID NO: 690 - DNA, 43 bp, Artificial Sequence
  primer
atgggaatgatgaacaaccccaattccaacccttagggaaccc

SEQ ID NO: 691 - DNA, 42 bp, Artificial Sequence
  primer
ccttgccctttaaggtggtggtgtccaacccttagggaaccc

SEQ ID NO: 692 - DNA, 47 bp, Artificial Sequence
  primer
ctgccttatgactcaagatgggagtttctccaacccttagggaaccc

SEQ ID NO: 693 - DNA, 42 bp, Artificial Sequence
  primer
gtgccagcaagatccaatctagagggccccgagaacctcgaa

SEQ ID NO: 694 - DNA, 45 bp, Artificial Sequence
  primer
gtgccagcaagatccaatctagagggcgacctcttccagaagctg

SEQ ID NO: 695 - DNA, 50 bp, Artificial Sequence
  primer
gtgccagcaagatccaatctagaatccaacgaccaagaactctctggaaa

SEQ ID NO: 696 - DNA, 48 bp, Artificial Sequence
  primer
gtgccagcaagatccaatctagagccagttatttgcctcagtaaaaga

SEQ ID NO: 697 - DNA, 53 bp, Artificial Sequence
  primer
gtgccagcaagatccaatctagacagtatgagtacacggagctcaagaaacag

SEQ ID NO: 698 - DNA, 48 bp, Artificial Sequence
  primer
gtgccagcaagatccaatctagacacaatgaaacagatttgcaaaaag

SEQ ID NO: 699 - DNA, 43 bp, Artificial Sequence
  primer
gtgccagcaagatccaatctagaccgcctgcctgcgcacctgc

SEQ ID NO: 700 - DNA, 45 bp, Artificial Sequence
  primer
gtgccagcaagatccaatctagacgggcagaaggctggtgacaag

SEQ ID NO: 701 - DNA, 43 bp, Artificial Sequence
  primer
gtgccagcaagatccaatctagaaagggccaaccactggggaa

SEQ ID NO: 702 - DNA, 53 bp, Artificial Sequence
  primer
gtgccagcaagatccaatctagaaaaacaagtgcacagacaacaccaagtaag

SEQ ID NO: 703 - DNA, 49 bp, Artificial Sequence
  primer
gtgccagcaagatccaatctagagggcaaatgagaacagcaacatacag

SEQ ID NO: 704 - DNA, 49 bp, Artificial Sequence
  primer
gtgccagcaagatccaatctagagcacacctctcaatgcagctttacag

SEQ ID NO: 705 - DNA, 52 bp, Artificial Sequence
  primer
gtgccagcaagatccaatctagatgaggacaagttctacagccacaagaaaa

SEQ ID NO: 706 - DNA, 44 bp, Artificial Sequence
  primer
gtgccagcaagatccaatctagaagaaccccaacagcaaagaag

SEQ ID NO: 707 - DNA, 45 bp, Artificial Sequence
  primer
gtgccagcaagatccaatctagacctaagatgcccgacttcaact

SEQ ID NO: 708 - DNA, 47 bp, Artificial Sequence
  primer
gtgccagcaagatccaatctagaaatgctggacttccgtagtgacca

SEQ ID NO: 709 - DNA, 53 bp, Artificial Sequence
  primer
gtgccagcaagatccaatctagacagtgccaggaaagagaattagagatcagt

SEQ ID NO: 710 - DNA, 46 bp, Artificial Sequence
  primer
gtgccagcaagatccaatctagacaaagcccacgctgaagcgaaag

SEQ ID NO: 711 - DNA, 49 bp, Artificial Sequence
  primer
gtgccagcaagatccaatctagaagctaaaaggacagcaggtgctacca

SEQ ID NO: 712 - DNA, 51 bp, Artificial Sequence
  primer
gtgccagcaagatccaatctagagttcagagactgaaggatgaagccagag

SEQ ID NO: 713 - DNA, 52 bp, Artificial Sequence
  primer
gtgccagcaagatccaatctagagatgatgatgaagaggatgatgatgaaga

SEQ ID NO: 714 - DNA, 44 bp, Artificial Sequence
  primer
gtgccagcaagatccaatctagacagcttccagtcacagcatag

SEQ ID NO: 715 - DNA, 51 bp, Artificial Sequence
  primer
gtgccagcaagatccaatctagagatgtcagcatacccagatccacattag

SEQ ID NO: 716 - DNA, 47 bp, Artificial Sequence
  primer
gtgccagcaagatccaatctagaaccgcttggaatgctgcaacaatg

SEQ ID NO: 717 - DNA, 50 bp, Artificial Sequence
  primer
gtgccagcaagatccaatctagaccaacaggcatgataggatatggaatt

SEQ ID NO: 718 - DNA, 48 bp, Artificial Sequence
  primer
gtgccagcaagatccaatctagactttggccctgtatcaggagcacag

SEQ ID NO: 719 - DNA, 44 bp, Artificial Sequence
  primer
gtgccagcaagatccaatctagactgggccagaatggtgaggag

SEQ ID NO: 720 - DNA, 46 bp, Artificial Sequence
  primer
gtgccagcaagatccaatctagaggctggcgcacctgggcgtgcag

SEQ ID NO: 721 - DNA, 45 bp, Artificial Sequence
  primer
gtgccagcaagatccaatctagatgatgaaggggaggaaggagag

SEQ ID NO: 722 - DNA, 51 bp, Artificial Sequence
  primer
gtgccagcaagatccaatctagacgtgatctttgatagatccagggaagag

SEQ ID NO: 723 - DNA, 50 bp, Artificial Sequence
  primer
gtgccagcaagatccaatctagattcttggctcaacaagccataaaacag

SEQ ID NO: 724 - DNA, 52 bp, Artificial Sequence
  primer
gtgccagcaagatccaatctagaggtcttataatcttccctctcttccggat

SEQ ID NO: 725 - DNA, 48 bp, Artificial Sequence
  primer
gtgccagcaagatccaatctagaccagcaaccttccatctctcatcag

SEQ ID NO: 726 - DNA, 47 bp, Artificial Sequence
  primer
gtgccagcaagatccaatctagaccgcagagcactgtatcacgaaaa

SEQ ID NO: 727 - DNA, 53 bp, Artificial Sequence
  primer
gtgccagcaagatccaatctagaattcctgttcctactacagttcctgttcct

SEQ ID NO: 728 - DNA, 52 bp, Artificial Sequence
  primer
gtgccagcaagatccaatctagaaccaagaggctattcaagatctctgcctg

SEQ ID NO: 729 - DNA, 52 bp, Artificial Sequence
  primer
gtgccagcaagatccaatctagaaccaagaggctattcaagatctctgtatg

SEQ ID NO: 730 - DNA, 53 bp, Artificial Sequence
  primer
gtgccagcaagatccaatctagaagcatttggttttaaattatggagtatgtt

SEQ ID NO: 731 - DNA, 45 bp, Artificial Sequence
  primer
tctgtggagacgagaatattctggtttccaacccttagggaaccc

SEQ ID NO: 732 - DNA, 50 bp, Artificial Sequence
  primer
gtgccagcaagatccaatctagagatttgtgattttggtctagccagagt

SEQ ID NO: 733 - DNA, 50 bp, Artificial Sequence
  primer
catcaagaatgattctaattatgtggttaaatccaacccttagggaaccc

SEQ ID NO: 734 - DNA, 30 bp, Artificial Sequence
  primer
ggagcggacatggactacgactcgtaccag

SEQ ID NO: 735 - DNA, 30 bp, Artificial Sequence
  primer
agaatgaagaaattgatgttgtgacagtag

SEQ ID NO: 736 - DNA, 26 bp, Artificial Sequence
  primer
aggaaggcatcaaccacgagtgtaag

SEQ ID NO: 737 - DNA, 28 bp, Artificial Sequence
  primer
cagctctctgtaatgcgatactcaccag

SEQ ID NO: 738 - DNA, 30 bp, Artificial Sequence
  primer
aagagaggctgtatctccatgccagagcag

SEQ ID NO: 739 - DNA, 30 bp, Artificial Sequence
  primer
acccaaaagcagaccttggagaacagtcag

SEQ ID NO: 740 - DNA, 30 bp, Artificial Sequence
  primer
tctttgaactactgccggagcttctgccag

SEQ ID NO: 741 - DNA, 24 bp, Artificial Sequence
  primer
caccgaggaccagctcaatgacag

SEQ ID NO: 742 - DNA, 49 bp, Artificial Sequence
  primer
ggagcggacatggactacgactcgtaccagtccaacccttagggaaccc

SEQ ID NO: 743 - DNA, 49 bp, Artificial Sequence
  primer
agaatgaagaaattgatgttgtgacagtagtccaacccttagggaaccc

SEQ ID NO: 744 - DNA, 45 bp, Artificial Sequence
  primer
aggaaggcatcaaccacgagtgtaagtccaacccttagggaaccc

SEQ ID NO: 745 - DNA, 51 bp, Artificial Sequence
  primer
gtgccagcaagatccaatctagacagctctctgtaatgcgatactcaccag

SEQ ID NO: 746 - DNA, 53 bp, Artificial Sequence
  primer
gtgccagcaagatccaatctagaaagagaggctgtatctccatgccagagcag

SEQ ID NO: 747 - DNA, 53 bp, Artificial Sequence
  primer
gtgccagcaagatccaatctagaacccaaaagcagaccttggagaacagtcag

SEQ ID NO: 748 - DNA, 53 bp, Artificial Sequence
  primer
gtgccagcaagatccaatctagatctttgaactactgccggagcttctgccag

SEQ ID NO: 749 - DNA, 47 bp, Artificial Sequence
  primer
gtgccagcaagatccaatctagacaccgaggaccagctcaatgacag

SEQ ID NO: 750 - DNA, 22 bp, Artificial Sequence
  primer
catcccagtgactgcatccctc

SEQ ID NO: 751 - DNA, 20 bp, Artificial Sequence
  primer
ggggaccccattcccgagga

SEQ ID NO: 752 - DNA, 27 bp, Artificial Sequence
  primer
gctctccacagatagagaacatccagc

SEQ ID NO: 753 - DNA, 25 bp, Artificial Sequence
  primer
ctgaacagatgggtaaggatggcag

SEQ ID NO: 754 - DNA, 23 bp, Artificial Sequence
  primer
ggaccaaccacttcctaccccag

SEQ ID NO: 755 - DNA, 20 bp, Artificial Sequence
  primer
gccccaggtgtacccaccac

SEQ ID NO: 756 - DNA, 20 bp, Artificial Sequence
  primer
gcctcacctgcagatgcccc

SEQ ID NO: 757 - DNA, 25 bp, Artificial Sequence
  primer
gcaacctccaagtcccagatcatgt

SEQ ID NO: 758 - DNA, 20 bp, Artificial Sequence
  primer
ggagttcctggtcggctccg

SEQ ID NO: 759 - DNA, 27 bp, Artificial Sequence
  primer
cgagttcaagcaggcctatatcacctg

SEQ ID NO: 760 - DNA, 20 bp, Artificial Sequence
  primer
gtgggcctcctgggcctcag

SEQ ID NO: 761 - DNA, 23 bp, Artificial Sequence
  primer
tccctggaatgaagggacacaga

SEQ ID NO: 762 - DNA, 20 bp, Artificial Sequence
  primer
atggcaaaactggcccccct

SEQ ID NO: 763 - DNA, 23 bp, Artificial Sequence
  primer
tccctggacctaaaggtgctgct

SEQ ID NO: 764 - DNA, 23 bp, Artificial Sequence
  primer
aagcaggcaaacctggtgaacag

SEQ ID NO: 765 - DNA, 22 bp, Artificial Sequence
  primer
tccagggcctaagggtgacaga

SEQ ID NO: 766 - DNA, 20 bp, Artificial Sequence
  primer
ctggtgcccctggtgacaag

SEQ ID NO: 767 - DNA, 20 bp, Artificial Sequence
  primer
ctggaccccctggccccatt

SEQ ID NO: 768 - DNA, 20 bp, Artificial Sequence
  primer
agggtccccctggccctcct

SEQ ID NO: 769 - DNA, 20 bp, Artificial Sequence
  primer
ctggtcctgctggtccccga

SEQ ID NO: 770 - DNA, 20 bp, Artificial Sequence
  primer
ctggcgagcctggagcttca

SEQ ID NO: 771 - DNA, 22 bp, Artificial Sequence
  primer
tgtcctccttgaagggctccag

SEQ ID NO: 772 - DNA, 27 bp, Artificial Sequence
  primer
cctccactgaagaagctgaaacaagag

SEQ ID NO: 773 - DNA, 25 bp, Artificial Sequence
  primer
gagagtctggatggacatttgcagg

SEQ ID NO: 774 - DNA, 20 bp, Artificial Sequence
  primer
tgcgaagccacctctcgcag

SEQ ID NO: 775 - DNA, 41 bp, Artificial Sequence
  primer
catcccagtgactgcatccctctccaacccttagggaaccc

SEQ ID NO: 776 - DNA, 39 bp, Artificial Sequence
  primer
ggggaccccattcccgaggatccaacccttagggaaccc

SEQ ID NO: 777 - DNA, 46 bp, Artificial Sequence
  primer
gctctccacagatagagaacatccagctccaacccttagggaaccc

SEQ ID NO: 778 - DNA, 44 bp, Artificial Sequence
  primer
ctgaacagatgggtaaggatggcagtccaacccttagggaaccc

SEQ ID NO: 779 - DNA, 42 bp, Artificial Sequence
  primer
ggaccaaccacttcctaccccagtccaacccttagggaaccc

SEQ ID NO: 780 - DNA, 39 bp, Artificial Sequence
  primer
gccccaggtgtacccaccactccaacccttagggaaccc

SEQ ID NO: 781 - DNA, 39 bp, Artificial Sequence
  primer
gcctcacctgcagatgcccctccaacccttagggaaccc

SEQ ID NO: 782 - DNA, 44 bp, Artificial Sequence
  primer
gcaacctccaagtcccagatcatgttccaacccttagggaaccc

SEQ ID NO: 783 - DNA, 39 bp, Artificial Sequence
  primer
ggagttcctggtcggctccgtccaacccttagggaaccc

SEQ ID NO: 784 - DNA, 50 bp, Artificial Sequence
  primer
gtgccagcaagatccaatctagacgagttcaagcaggcctatatcacctg

SEQ ID NO: 785 - DNA, 43 bp, Artificial Sequence
  primer
gtgccagcaagatccaatctagagtgggcctcctgggcctcag

SEQ ID NO: 786 - DNA, 46 bp, Artificial Sequence
  primer
gtgccagcaagatccaatctagatccctggaatgaagggacacaga

SEQ ID NO: 787 - DNA, 43 bp, Artificial Sequence
  primer
gtgccagcaagatccaatctagaatggcaaaactggcccccct

SEQ ID NO: 788 - DNA, 46 bp, Artificial Sequence
  primer
gtgccagcaagatccaatctagatccctggacctaaaggtgctgct

SEQ ID NO: 789 - DNA, 46 bp, Artificial Sequence
  primer
gtgccagcaagatccaatctagaaagcaggcaaacctggtgaacag

SEQ ID NO: 790 - DNA, 45 bp, Artificial Sequence
  primer
gtgccagcaagatccaatctagatccagggcctaagggtgacaga

SEQ ID NO: 791 - DNA, 43 bp, Artificial Sequence
  primer
gtgccagcaagatccaatctagactggtgcccctggtgacaag

SEQ ID NO: 792 - DNA, 43 bp, Artificial Sequence
  primer
gtgccagcaagatccaatctagactggaccccctggccccatt

SEQ ID NO: 793 - DNA, 43 bp, Artificial Sequence
  primer
gtgccagcaagatccaatctagaagggtccccctggccctcct

SEQ ID NO: 794 - DNA, 43 bp, Artificial Sequence
  primer
gtgccagcaagatccaatctagactggtcctgctggtccccga

SEQ ID NO: 795 - DNA, 43 bp, Artificial Sequence
  primer
gtgccagcaagatccaatctagactggcgagcctggagcttca

SEQ ID NO: 796 - DNA, 45 bp, Artificial Sequence
  primer
gtgccagcaagatccaatctagatgtcctccttgaagggctccag

SEQ ID NO: 797 - DNA, 50 bp, Artificial Sequence
  primer
gtgccagcaagatccaatctagacctccactgaagaagctgaaacaagag

SEQ ID NO: 798 - DNA, 48 bp, Artificial Sequence
  primer
gtgccagcaagatccaatctagagagagtctggatggacatttgcagg

SEQ ID NO: 799 - DNA, 43 bp, Artificial Sequence
  primer
gtgccagcaagatccaatctagatgcgaagccacctctcgcag